(19) United States
(12) Patent Application Publication
(10) Pub. No.: US 2010/0204210 A1
(43) Pub. Date: Aug. 12, 2010

(54) METHOD FOR TREATING PULMONARY DISEASES USING RHO KINASE INHIBITOR COMPOUNDS

(76) Inventor: Scott Sorensen, (US)

Correspondence Address: HOWERY LLP, C/O IP DOCKETING DEPARTMENT, 2941 FAIRVIEW PARK DRIVE SUITE 200, FALLS CHURCH, VA, 22042, US

(21) Appl. No.: 12/630,779

(22) Filed: Dec. 3, 2009

Related U.S. Application Data
(60) Provisional application No. 61/119,999, filed on Dec. 4, 2008.

(51) Int. Cl. A61K 31/55; A61K 31/454; A61K 31/416; A61K 31/5377; A61K 31/496; A61K 31/4545; A61K 31/47; A61K 31/437; A61P 11/00

(52) U.S. Cl. 514/217.09; 514/322; 514/406; 514/234.5; 514/253.09; 514/318; 514/314; 514/300

(57) ABSTRACT

This invention relates to methods of treating pulmonary diseases in patients that beta adrenergic receptor agonist therapy is not effective. The method comprises the steps of: identifying a patient who suffers from a pulmonary disease and has reduced responsiveness to treatment with one or more beta adrenergic receptor agonists, and administering to the patient an effective amount of a Rho kinase inhibitor compound, wherein said pulmonary disease is selected from the group consisting of: asthma, chronic obstructive pulmonary disease, respiratory tract illness caused by respiratory syncytial virus infection such as RSV-induced wheezing, airway hyperreactivity, or bronchiolitis, bronchiectasis, alpha-1-antitrypsin deficiency, lymphangioleiomyomatosis, cystic fibrosis, bronchiolitis or wheezing caused by agents other than respiratory syncytial virus, chronic bronchitis, and occupational lung diseases.

The present application claims the benefit of U.S. Provisional Application No. 61/119,999, filed Dec. 4, 2008; which is incorporated herein by reference in its entirety.

### TECHNICAL FIELD

This invention relates to methods of treating pulmonary diseases or conditions for which beta adrenergic receptor agonist therapy or combined therapy with beta adrenergic receptor agonist and corticosteroid are not effective. Particularly, this invention relates to treating patients with pulmonary diseases, such as asthma, chronic obstructive pulmonary disease, and respiratory tract illness caused by respiratory syncytial virus infection such as RSV-induced wheezing, airway hyperreactivity, or bronchiolitis; such patients have reduced responsiveness to beta adrenergic receptor agonist therapy or combined therapy with beta adrenergic receptor agonist and corticosteroid. The method comprises administering to the patient a Rho kinase inhibitor compound.

### BACKGROUND OF THE INVENTION

### Asthma

Asthma is a common chronic disorder of the airways characterized by variable and recurring symptoms, reversible airway obstruction, bronchial hyperresponsiveness, and an underlying inflammation. Acute symptoms of asthma include cough, wheezing, shortness of breath and nocturnal awakening. These symptoms usually arise from bronchospasm and require bronchodilator therapy (see Expert Panel Report 3: Guidelines for the Diagnosis and Management of Asthma, NIH Publication No. 07-4051, Bethesda, Md.: U.S. Department of Health and Human Services; National Institutes of Health; National Heart, Lung, and Blood Institute; National Asthma Education and Prevention Program, (2007) and references therein).

Central to the pathophysiology of asthma is the presence of underlying airway inflammation mediated by the recruitment and activation of multiple cell types including mast cells, eosinophils, T lymphocytes, macrophages, dendritic cells and neutrophils. Type 2 T-helper (Th2) cells appear to play a central role in the activation of the immune cascade that results in inflammation. Th2-derived cytokines include IL-5, which is needed for eosinophil differentiation and survival, and IL-4 which is important for Th2 cell differentiation and with IL-13 is important for IgE formation and leads to overproduction of IgE and eosinophilia. IgE-driven activation of mucosal mast cells releases bronchoconstrictor mediators such as histamine and cysteinyl-leukotrienes as well as pro-inflammatory cytokines. Eosinophils contain inflammatory enzymes, generate leukotrienes, and express a wide variety of pro-inflammatory cytokines. Airway epithelial cells also play a role in the inflammatory process via release of cytokines such as eotaxin that direct and modify the inflammatory response. Acute and chronic inflammation can affect not only the airway caliber and airflow but also can increase the existing bronchial hyperresponsiveness to a variety of stimuli, which enhances susceptibility to bronchospasm.

As a consequence of airway inflammation and the generation of growth factors, the airway smooth muscle cell can undergo proliferation, activation, contraction, and hypertrophy events that can influence airway airflow limitation. In asthma, the dominant physiological event leading to clinical symptoms is airway narrowing and a subsequent interference with airflow. In acute exacerbations of asthma, bronchial smooth muscle contraction (bronchoconstriction) occurs quickly to narrow the airways in response to exposure to a variety of stimuli including allergens or irritants. Allergen-induced acute bronchoconstriction results from an IgE-dependent release of mediators from mast cells that includes histamine, tryptase, leukotrienes, and prostaglandins that directly contract airway smooth muscle. The mechanisms influencing airway hyperresponsiveness are multiple and include inflammation, dysfunctional neuroregulation, and airway remodeling. Airway remodeling involves structural changes including thickening of the sub-basement membrane, subepithelial fibrosis, airway smooth muscle hypertrophy and hyperplasia, blood vessel proliferation and dilation with consequent permanent changes in the airway that increase airflow obstruction and that is not prevented by or fully reversible by current therapies.

Current therapies for asthma include either beta adrenergic receptor agonists alone or in combination with corticosteroids. Beta adrenergic receptor agonists act as bronchodilators while corticosteroids act to decrease inflammation. Most patients have mild to moderate asthma controlled with either an inhaled beta adrenergic receptor agonist alone or in combination with an inhaled corticosteroid. However, a subset of patients exists in whom high doses of both beta adrenergic receptor agonists and corticosteroid drugs fail to provide control of the disease (Bateman E D et al Am J Respir Crit Care Med 170:836-844 (2004)). It is estimated that 5 to 10% of asthmatics have symptomatic disease despite maximum treatment with combined anti-inflammatory and bronchodilator drugs. In addition, the regular use of beta adrenergic receptor agonists can result in a loss of effectiveness over time and high doses of short acting beta agonists may be detrimental to control of asthma (Chanez P J Allergy Clin Immunol 119:1337-1348 (2007)).

### Chronic Obstructive Pulmonary Disease

Chronic obstructive pulmonary disease (COPD) is the most common chronic lung disease associated with significant morbidity and mortality. In the United States, COPD is the fourth leading cause of death and accounts for more than $30 billion in annual health care costs. An estimated 16 million adults are affected by COPD, and each year ˜120,000 Americans die of the disease. COPD is defined as a chronic disease characterized by airway/alveolar/systemic inflammation, with measured airflow obstruction (FEV₁/FVC<70% and FEV₁<80% predicted) that is only partially improved with bronchodilator therapy. The local and systemic release of inflammatory mediators by the lung cells leads to airway disease (chronic obstructive bronchitis) and, in a minority of patients, to destruction of parenchymal tissue (emphysema), both of which can result in the airflow limitation that characterizes COPD (see Doherty D E et al, Clin Cornerstone 6:S5-16 (2004) and MacNee, Clin Ches Med 28:479-513 (2007.

The chronic inflammation, airway obstruction, and tissue damage that occur in COPD all result from chronic exposure to inhaled toxic substances, primarily cigarette smoke. In response to the chemical insult of inhaled tobacco smoke, inflammatory cells (including macrophages, neutrophils, and T-lymphocytes, primarily CD8 lymphocytes) are activated in the small and large airways as well as in the lung parenchyma. These activated inflammatory cells release a host of cytokines and other mediators (including tumor necrosis factor-α, interleukin [IL]-8 and leukotriene B₄), which can cause damage to lung tissue. The end result of the release of these cytokines and mediators may be the development of chronic inflammation of the airways, mucus gland hypertrophy and goblet-cell hyperplasia with increased mucus secretion, fibrosis and narrowing of smaller airways, destruction of the parenchyma (the connective tissue/cells in the lungs), and changes in the blood vessels that may result in the development of pulmonary hypertension. These pathologic changes manifest themselves as mucus hypersecretion, limited airflow, hyperinflation, and gas exchange abnormalities which are the major physiologic abnormalities that characterize COPD. A loss in the integrity of the lung's connective tissue leads to a decrease of elastic recoil and hyperinflation.

Current therapies to treat COPD include bronchodilators that help to some degree to decrease hyperinflation, therefore increasing inspiratory capacity and relieving dyspnea. Although corticosteroids are an effective treatment for most cases of asthma, the inflammatory cells and mediators in COPD are not sensitive to treatment with systemic or inhaled corticosteroids, thus making treatment with these agents of limited usefulness in COPD.

### RSV Infection

Respiratory syncytial virus (RSV) causes acute respiratory tract illness in persons of all ages. RSV is a leading cause of lower respiratory tract infection (LRTI) in children younger than 2 years. It is associated with up to 120,000 pediatric hospitalizations each year, and is increasing in frequency. RSV also is a significant cause of morbidity and mortality from LRTI in elderly patients (Collins et al., J Virol 82:2040-2055 (2008); Peebles et al., Proc Am Thorac Soc 2:110-115 (2005)).

After replicating in the nasopharynx, RSV infects the small bronchiolar epithelium and extends to the type 1 and 2 alveolar pneumocytes in lung. Pathologic findings of RSV include necrosis of epithelial cells, occasional proliferation of the bronchiolar epithelium, infiltrates of monocytes and T cells centered on bronchial and pulmonary arterioles, and neutrophils between the vascular structures and small airways. This leads to airway obstruction, air trapping and increased airway resistance, and also is associated with a finding of neutrophilia in bronchoalveolar lavage. The immune response to RSV, especially cytokine and chemokine release, appears to play a role in the pathogenesis and severity of bronchiolitis. There is a distinct pattern of cytokines and chemokines induced by RSV infection and some have been associated with disease severity. The cytokines IL-8, IL-6, TNF-alpha, and IL-1 beta can be detected in airway secretions of infected children (Smyth et al. Arch Dis Child 76:210 (1997)), and IL-6 levels correlate with severe disease. Chemokines identified in respiratory tract secretions of children include CCL3, CCL2, CCL11 and CCL5, but only the beta-chemokines, particularly MIP-1 alpha, are associated with severe disease (Welliver et al. Pediatr Infect Dis J 21:457 (2002)).

RSV can involve both lower and upper respiratory tract. Severe lower respiratory tract disease can involve bronchiolitis, bronchospasm, pneumonia, and acute respiratory failure in children. Lower respiratory tract involvement usually occurs with primary infection, and may occur in second infections and can cause wheezing, tachypnea and apnea. Repeat RSV infections occur frequently in children and young adults and result in significant upper respiratory tract symptoms. Signs include cough, coryza, rhinorrhea, and conjunctivitis. RSV infection in adults also may cause short-term airway reactivity.

There is no direct treatment for RSV infection and the respiratory complications it causes. The current therapy for RSV is primarily supportive. Bronchodilator therapy in infants with bronchiolitis, largely caused by RSV infection, did not demonstrate benefit in large randomized trials and systematic reviews.

### Other Pulmonary Diseases

Bronchoconstriction is a key feature of multiple other respiratory diseases. These diseases include bronchiectasis, alpha-1-antitrypsin deficiency (AATD), lymphangioleiomyomatosis (LAM), cystic fibrosis, bronchiolitis/wheezing, chronic bronchitis, and occupational lung diseases such as coal workers' pneumoconiosis, byssinosis (brown lung disease), asbestosis and silicosis. These diseases are often treated by the administration of beta adrenergic receptor agonists either alone or in combination with a corticosteroid. Current therapies are not particularly effective in treating these diseases. In addition, responsiveness to bronchodilators does not always persist in these patients.

### Beta Agonists as a Treatment

The beta adrenergic receptors belong to the superfamily of G protein coupled receptors that transduce responses via activation of an intermediary G protein. β₂ adrenergic receptors are the predominant subtype expressed in lung where these receptors mediate the relaxant effects of adrenergic agonists on smooth muscle. Beta adrenergic receptors are linked by G_{S} to the activation of adenylyl cyclase which increases intracellular cAMP. Subsequent action of cAMP dependent protein kinase results in the phosphorylation of multiple proteins such as Rho kinase and myosin light chain kinase. Inactivation of myosin light chain kinase and Rho kinase results in a decrease in the phosphorylation state of the myosin regulatory light chain which results in relaxation. In contrast, smooth muscle contractile agents, such as acetylcholine, stimulate the calcium-dependent activation of myosin light chain kinase and Rho kinase. Therefore, the tone of smooth muscle is regulated by the convergent activity of these pathways on the phosphorylation state of myosin, cAMP favoring relaxation and calcium favoring constriction. In addition to the effects on airway smooth muscle, beta adrenergic receptors may play a role in regulating the inflammatory response in asthma. Notably, beta adrenergic receptors are effective at inhibiting the in vitro activation of human mast cells, migration and chemotaxis of inflammatory cells, and cytokine release from monocytes. The clinical significance of these effects remains unclear since beta adrenergic agonist do not inhibit inflammatory cell influx or the late asthmatic response in vivo (Broadley K J Eur J Pharmacol 533:15-27 (2006)).

Prolonged exposure to beta adrenergic receptor agonists can result in a loss of responsiveness to the beta adrenergic receptor agonist itself, a process termed homologous desensitization. Homologous desensitization is attributed to several functionally distinct adaptive changes. Upon receptor activation, phosphorylation of the receptor by specific G protein receptor kinases results in the functional uncoupling of the receptor from the cognate G protein. The receptor is then trafficked away from the plasma membrane to endosomal compartments. Prolonged agonist exposure results in the targeting of endosomal receptors to lysosomes where the receptor undergoes degradation and the total number of cellular receptors is decreased. In addition, adaptive changes to the signaling pathways that are recruited by beta adrenergic receptors can further limit the efficacy of these agents.

Heterologous desensitization of beta adrenergic receptor signaling occurs when beta adrenergic receptor-independent factors limit the bronchorelaxant effect of beta adrenergic receptor agonists. One example of this is the activity of contractile agents such as acetylcholine, histamine, leukotrienes and prostaglandins that activate Gq pathways to increase intracellular calcium. These agents enhance smooth muscle contractility and can overcome the effects of beta adrenergic receptor-mediated bronchorelaxation. In addition, activation of Gq pathways can lead to protein kinase C-mediated phosphorylation and inactivation of beta adrenergic receptors. This phosphorylation can serve to further limit the efficacy of beta adrenergic receptor agonists. In another form of heterologous desensitization, pro-inflammatory cytokines such as TNFalpha and IL-1beta, have been shown to reduce beta adrenergic receptor responsiveness through disruption of beta-adrenergic receptor signaling pathways (Koto et al., J Clin Invest 98:1780-1787 (1996); Hakonarson H et al J Clin Invest 97:2593-2600 (1996)). In patients with steroid resistance in whom corticosteroid administration fails to reduce the underlying inflammation including levels of TNF-α and IL-1β, this mechanism is likely to contribute or cause reduced responsiveness to beta adrenergic receptor therapy.

Desensitization at a cellular level underlies the clinical observations of tolerance whereby a drug substance loses activity with repeated use. In respiratory conditions such as asthma, COPD, bronchiectasis, alpha-1-antitrypsin deficiency (AATD), lymphangioleiomyomatosis (LAM), cystic fibrosis, bronchiolitis/wheezing, chronic bronchitis, and occupational lung diseases such as coal workers' pneumoconiosis, byssinosis (brown lung disease), asbestosis and silicosis, regular use of beta agonists can result in a loss of effectiveness and in some rare instances can even worsen control of asthma. For example, regular use of beta adrenergic receptor agonists causes a marked step-down in bronchoprotection against inhaled methacholine or histamine (Anderson G P Clin Rev Allergy Immunol 31:119-130 (2006)).

Beta adrenergic receptor agonists can be administered in combination with corticosteroids. For both asthma and COPD, short-acting beta adrenergic receptor agonists represent first-line therapy and corticosteroids are often added as these diseases progress. The combination of long-acting beta adrenergic receptor agonists with corticosteroids to treat moderate to severe asthma has been demonstrated to improve control of airway diseases when compared to either agent alone in some patients. However, a subset of patients exists in whom high doses of both beta adrenergic receptor agonists and corticosteroid drugs fail to provide control of asthma (Bateman E D et al Am J Respir Crit Care Med 170:836-844 (2004)). It is estimated that 5 to 10% of asthmatics have symptomatic disease despite maximum treatment with combined anti-inflammatory and beta adrenergic receptor agonist drugs. The lack of responsiveness to beta adrenergic receptor agonists in this subset of patients leads to uncontrolled bronchoconstriction.

### Rho Kinase

The Rho family of small GTP binding proteins can be activated by several extracellular stimuli such as growth factors, hormones and mechanic stress and function as a molecular signaling switch by cycling between an inactive GDP-bound form and an active GTP-bound form to elicit cellular responses. Rho kinase (ROCK) functions as a key downstream mediator of Rho and exists as two isoforms (ROCK 1 and ROCK 2) that are ubiquitously expressed. ROCKs are serine/threonine kinases that regulate the function of a number of substrates including cytoskeletal proteins such as adducin, moesin, Na⁺—H⁺ exchanger 1 (NHE1), LIM-kinase and vimentin, contractile proteins such as the myosin light chain phosphatase binding subunit (MYPT-1), CPI-17, myosin light chain and calponin, microtubule associated proteins such as Tau and MAP-2, neuronal growth cone associate proteins such as CRMP-2, signaling proteins such as PTEN and transcription factors such as serum response factor (Loirand et al, Circ Res 98:322-334 (2006)). As a key intermediary of multiple signaling pathways, ROCK regulates a diverse array of cellular phenomena including cytoskeletal rearrangement, actin stress fiber formation, proliferation, chemotaxis, cytokinesis, cytokine and chemokine secretion, endothelial or epithelial cell junction integrity, apoptosis, transcriptional activation and smooth muscle contraction (WO 2005/003101A2, WO 2005/034866A2).

There is a sizable group of asthma patients, treated with beta adrenergic receptor agonists alone or in combination with inhaled corticososteroids, who have uncontrolled asthma. There is a need for an effective treatment in patients having pulmonary diseases who have reduced responsiveness to beta adrenergic receptor agonist treatment.

### SUMMARY OF THE INVENTION

This invention relates to methods of treating pulmonary diseases or conditions for which beta adrenergic receptor agonist therapy or combined therapy with beta adrenergic receptor agonists and corticosteroids are not effective.

The present invention provides a method for treating pulmonary diseases in patients who have reduced responsiveness to treatment with one or more beta adrenergic receptor agonists, or who has reduced responsiveness to the combined treatment with beta adrenergic receptor agonists and corticosteroids. The method comprises the steps of: (a) identifying a patient who suffers from a pulmonary disease and has reduced responsiveness to treatment with one or more beta adrenergic receptor agonists or to the combined treatment with beta adrenergic receptor agonists and corticosteroids, and (b) administering to the patient an effective amount of a Rho kinase inhibitor compound. The reduced responsiveness can be due to tolerance (desensitization) developed in the patient to the treatment of the beta adrenergic receptor agonists. The reduced responsiveness can also be due to viral infection, bacterial infection, allergen exposure, an increase in inflammation, or corticosteroid resistance leading to uncontrolled inflammation, treatment with beta adrenergic receptor antagonists (beta blockers), workplace exposure to sensitizing chemicals, environmental exposure to irritants such as tobacco smoke, sulfite sensitivity, or some unknown reason.

In one embodiment, the patient has been treated with a corticosteroid in combination with the one or more beta adrenergic receptor agonists. In another embodiment, the patient has not been treated with a corticosteroid in combination with the one or more beta adrenergic receptor agonists. In either case, the patient has reduced responsiveness to the beta adrenergic receptor agonist treatment and requires a different treatment.

The present invention also provides a method for treating pulmonary diseases in patients who had reduced responsiveness to treatment with beta adrenergic receptor agonists but has regained responsiveness to the beta adrenergic receptor agonist after a combined treatment with the beta adrenergic receptor agonist and a corticosteroid. The method comprises the steps of: identifying such patient and administering to the patient an effective amount of a Rho kinase inhibitor compound.

Pulmonary diseases suitable to be treated by the present invention include asthma, chronic obstructive pulmonary disease, respiratory tract illness caused by respiratory syncytial virus infection such as RSV-induced wheezing, airway hyperreactivity, or bronchiolitis, bronchiectasis, alpha-1-antitrypsin deficiency (AATD), lymphangioleiomyomatosis (LAM), cystic fibrosis, bronchiolitis or wheezing caused by agents other than respiratory syncytial virus, chronic bronchitis, and occupational lung diseases such as coal workers' pneumoconiosis, byssinosis (brown lung disease), asbestosis, and silicosis. The active compound is delivered to a subject either by systemic administration or local administration.

### BRIEF DESCRIPTION OF THE DRAWINGS

FIGS. 1A-1E show changes in tension over time in rat tracheal rings. Application of 300 nM carbachol (CCh) results in contraction of the tracheal rings as measured by an increase in tension. Application of the beta-adrenergic receptor agonist isoproterenol (FIG. 1A), formoterol (FIG. 1B) or albuterol (FIG. 1C) to precontracted trachea results in an initial relaxation of the tension followed by a fade of beta adrenergic receptor responses to a more contractile state. Application of ROCK inhibitors (FIGS. 1D and 1E) to precontracted trachea results in a fully efficacious and prolonged relaxant response.

FIG. 2 shows representative traces demonstrating the efficacy of compound 7 in tracheal rings that have reduced responsiveness to beta-adrenergic receptor agonists. After contraction of trachea with 300 nM CCh, application of 3 μM formoterol induces an initial relaxant response followed by a fade of the response to a more contractile state. Subsequent application of 3 μM formoterol (A) or 100 μM albuterol (B) is less effective in restoring relaxation while application of 3 μM compound 7 results in relaxation of the tissue.

FIG. 3 shows representative traces demonstrating the efficacy of compound 16 in tracheal rings that have reduced responsiveness to beta-adrenergic receptor agonist. After contraction of trachea with 300 nM CCh, application of 3 μM formoterol induces an initial relaxant response followed by a fade of the response to a more contractile state. Subsequent application of 3 μM formoterol (A) or 100 μM albuterol (B) is less effective in restoring relaxation while application of 3 μM compound 16 results in relaxation of the tissue.

FIG. 4 shows the quantitation of multiple experiments in which carbachol-precontracted tissue is treated with 3 μM formoterol followed by (A) 3 μM formoterol and 3 μM compound 7 or (B) 100 μM albuterol and 3 μM compound 7. Four parameters were analyzed for quantitation of efficacies: i) the maximal relaxation induced by initial formoterol treatment, ii) the fade of the response to the initial formoterol treatment, iii) the maximal relaxation induced by the second addition of a beta agonist, and iv) the maximal relaxation induced by Rho kinase inhibitor. Data shown are mean±SEM for 4 to 5 replicate experiments.

FIG. 5 shows the quantitation of multiple experiments in which carbachol-precontracted tissue is treated with 3 μM formoterol followed by (A) 3 μM formoterol and 3 μM compound 11 or (B) 100 μM albuterol and 3 μM compound 11. Four parameters were analyzed for quantitation of efficacies: i) the maximal relaxation induced by initial formoterol treatment, ii) the fade of the response to the initial formoterol treatment, iii) the maximal relaxation induced by the second addition of a beta agonist, and iv) the maximal relaxation induced by Rho kinase inhibitor. Data shown are mean±SEM for 4 to 5 replicate experiments.

FIG. 6 shows the quantitation of multiple experiments in which carbachol-precontracted tissue is treated with 3 μM formoterol followed by (A) 3 μM formoterol and 3 μM compound 16 or (B) 100 μM albuterol and 3 μM compound 16. Four parameters were analyzed for quantitation of efficacies: i) the maximal relaxation induced by initial formoterol treatment, ii) the fade of the response to the initial formoterol treatment, iii) the maximal relaxation induced by the second addition of a beta agonist, and iv) the maximal relaxation induced by Rho kinase inhibitor. Data shown are mean±SEM for 4 to 5 replicate experiments.

FIG. 7 shows the quantitation of multiple experiments in which carbachol-precontracted tissue is treated with 3 μM formoterol followed by (A) 3 μM formoterol and 3 μM compound 10 or (B) 100 μM albuterol and 3 μM compound 10. Four parameters were analyzed for quantitation of efficacies: i) the maximal relaxation induced by initial formoterol treatment, ii) the fade of the response to the initial formoterol treatment, iii) the maximal relaxation induced by the second addition of a beta agonist, and iv) the maximal relaxation induced by Rho kinase inhibitor. Data shown are mean±SEM for 4 to 5 replicate experiments.

FIG. 8 shows the dose response curve for isoproterenol, a beta adrenergic receptor agonist, to induce relaxation in rat tracheal rings pretreated with either vehicle alone or the pro-inflammatory cytokines, IL-1β and TNF-α. Data are reported as a percent of the maximal carbachol (300 nM carbachol) response. *, p<0.05 for the comparable dose of isoproterenol from vehicle-pretreated tissues using Student's t-test.

FIG. 9 shows the dose response curve for albuterol, a beta adrenergic receptor agonist, to induce relaxation in rat tracheal rings pretreated with either vehicle alone or the pro-inflammatory cytokines, IL-1β and TNF-α. Data are reported as a percent of the maximal carbachol (300 nM carbachol) response. *, p<0.05 for the comparable dose of albuterol from vehicle-pretreated tissues using Student's t-test.

FIG. 10 shows the dose response curves for compound 7 to induce relaxation in rat tracheal rings pretreated with either vehicle alone or the pro-inflammatory cytokines, IL-1β and TNF-α. Data are reported as a percent of the maximal carbachol (300 nM carbachol) response.

### DETAILED DESCRIPTION OF THE INVENTION

### Definitions

When present, unless otherwise specified, the following terms are generally defined as, but are not limited to, the following:

“Alkyl” refers to groups of from 1 to 12 carbon atoms inclusively, either straight chained or branched, more preferably from 1 to 8 carbon atoms inclusively, and most preferably 1 to 6 carbon atoms inclusively.

“Alkenyl” refers to groups of from 2 to 12 carbon atoms inclusively, either straight or branched containing at least one double bond but optionally containing more than one double bond.

“Alkynyl” refers to groups of from 2 to 12 carbon atoms inclusively, either straight or branched containing at least one triple bond but optionally containing more than one triple bond, and additionally optionally containing one or more double bonded moieties.

“Alkoxy” refers to the group alkyl-O— wherein the alkyl group is as defined above including optionally substituted alkyl groups as also defined above.

“Alkenoxy” refers to the group alkenyl-O— wherein the alkenyl group is as defined above including optionally substituted alkenyl groups as also defined above.

“Alkynoxy” refers to the group alkynyl-O— wherein the alkynyl group is as defined above including optionally substituted alkynyl groups as also defined above.

“Aryl” refers to an unsaturated aromatic carbocyclic group of from 6 to 14 carbon atoms inclusively having a single ring (e.g., phenyl) or multiple condensed rings (e.g., naphthyl or anthryl). Preferred aryls include phenyl, naphthyl and the like.

“Arylalkyl” refers to aryl-alkyl-groups preferably having from 1 to 6 carbon atoms inclusively in the alkyl moiety and from 6 to 10 carbon atoms inclusively in the aryl moiety. Such arylalkyl groups are exemplified by benzyl, phenethyl and the like.

“Arylalkenyl” refers to aryl-alkenyl-groups preferably having from 2 to 6 carbon atoms in the alkenyl moiety and from 6 to 10 carbon atoms inclusively in the aryl moiety.

“Arylalkynyl” refers to aryl-alkynyl-groups preferably having from 2 to 6 carbon atoms inclusively in the alkynyl moiety and from 6 to 10 carbon atoms inclusively in the aryl moiety.

“Cycloalkyl” refers to cyclic alkyl groups of from 3 to 12 carbon atoms inclusively having a single cyclic ring or multiple condensed rings which can be optionally substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple ring structures such as adamantyl, and the like.

“Cycloalkenyl” refers to cyclic alkenyl groups of from 4 to 12 carbon atoms inclusively having a single cyclic ring or multiple condensed rings and at least one point of internal unsaturation, which can be optionally substituted with from 1 to 3 alkyl groups. Examples of suitable cycloalkenyl groups include, for instance, cyclobut-2-enyl, cyclopent-3-enyl, cyclooct-3-enyl and the like.

“Cycloalkylalkyl” refers to cycloalkyl-alkyl-groups preferably having from 1 to 6 carbon atoms inclusively in the alkyl moiety and from 6 to 10 carbon atoms inclusively in the cycloalkyl moiety. Such cycloalkylalkyl groups are exemplified by cyclopropylmethyl, cyclohexylethyl and the like.

“Cycloalkylalkenyl” refers to cycloalkyl-alkenyl-groups preferably having from 2 to 6 carbon atoms inclusively in the alkenyl moiety and from 6 to 10 carbon atoms inclusively in the cycloalkyl moiety. Such cycloalkylalkenyl groups are exemplified by cyclohexylethenyl and the like.

“Cycloalkylalkynyl” refers to cycloalkyl-alkynyl-groups preferably having from 2 to 6 carbon atoms inclusively in the alkynyl moiety and from 6 to 10 carbon atoms inclusively in the cycloalkyl moiety. Such cycloalkylalkynyl groups are exemplified by cyclopropylethynyl and the like.

“Halo” substituents refer to fluorine, chlorine, bromine, and iodine.

“Heteroaryl” refers to a monovalent aromatic heterocyclic group of from 1 to 10 carbon atoms inclusively and 1 to 4 heteroatoms inclusively selected from oxygen, nitrogen and sulfur within the ring. Such heteroaryl groups can have a single ring (e.g., pyridyl or furyl) or multiple condensed rings (e.g., indolizinyl or benzothienyl).

“Heteroarylalkyl” refers to heteroaryl-alkyl-groups preferably having from 1 to 6 carbon atoms inclusively in the alkyl moiety and from 6 to 10 atoms inclusively in the heteroaryl moiety. Such heteroarylalkyl groups are exemplified by pyridylmethyl and the like.

“Heteroarylalkenyl” refers to heteroaryl-alkenyl-groups preferably having from 2 to 6 carbon atoms inclusively in the alkenyl moiety and from 6 to 10 atoms inclusively in the heteroaryl moiety.

“Heteroarylalkynyl” refers to heteroaryl-alkynyl-groups preferably having from 2 to 6 carbon atoms inclusively in the alkynyl moiety and from 6 to 10 atoms inclusively in the heteroaryl moiety.

“Heterocycle” refers to a saturated or unsaturated group having a single ring or multiple condensed rings, from 1 to 8 carbon atoms inclusively and from 1 to 4 hetero atoms inclusively selected from nitrogen, sulfur or oxygen within the ring. Such heterocyclic groups can have a single ring (e.g., piperidinyl or tetrahydrofuryl) or multiple condensed rings (e.g., indolinyl, dihydrobenzofuran or quinuclidinyl). Preferred heterocycles include piperidinyl, pyrrolidinyl and tetrahydrofuryl.

“Heterocycle-alkyl” refers to heterocycle-alkyl-groups preferably having from 1 to 6 carbon atoms inclusively in the alkyl moiety and from 6 to 10 atoms inclusively in the heterocycle moiety. Such heterocycle-alkyl groups are exemplified by morpholino-ethyl, pyrrolidinylmethyl, and the like.

“Heterocycle-alkenyl” refers to heterocycle-alkenyl-groups preferably having from 2 to 6 carbon atoms inclusively in the alkenyl moiety and from 6 to 10 atoms inclusively in the heterocycle moiety.

“Heterocycle-alkynyl” refers to heterocycle-alkynyl-groups preferably having from 2 to 6 carbon atoms inclusively in the alkynyl moiety and from 6 to 10 atoms inclusively in the heterocycle moiety.

Examples of heterocycles and heteroaryls include, but are not limited to, furan, thiophene, thiazole, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, pyrrolidine, indoline and the like.

Unless otherwise specified, positions occupied by hydrogen in the foregoing groups can be further substituted with substituents exemplified by, but not limited to, hydroxy, oxo, nitro, methoxy, ethoxy, alkoxy, substituted alkoxy, trifluoromethoxy, haloalkoxy, fluoro, chloro, bromo, iodo, halo, methyl, ethyl, propyl, butyl, alkyl, alkenyl, alkynyl, substituted alkyl, trifluoromethyl, haloalkyl, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, phenyl, aryl, substituted aryl, aryloxy, arylalkyl, arylalkenyl, arylalkynyl, pyridyl, imidazolyl, heteroaryl, substituted heteroaryl, heteroaryloxy, heteroarylalkyl, heteroarylalkenyl, heteroarylalkynyl, cyclopropyl, cyclobutyl, cyclopentyl, cyclohexyl, cycloalkyl, cycloalkenyl, cycloalkylalkyl, substituted cycloalkyl, cycloalkyloxy, pyrrolidinyl, piperidinyl, morpholino, heterocycle, (heterocycle)oxy, and (heterocycle)alkyl; and preferred heteroatoms are oxygen, nitrogen, and sulfur. It is understood that where open valences exist on these substituents they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further understood that the above substitutions can be made provided that replacing the hydrogen with the substituent does not introduce unacceptable instability to the molecules of the present invention, and is otherwise chemically reasonable.

The term “heteroatom-containing substituent” refers to substituents containing at least one non-halogen heteroatom. Examples of such substituents include, but are not limited to, hydroxy, oxo, nitro, methoxy, ethoxy, alkoxy, substituted alkoxy, trifluoromethoxy, haloalkoxy, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, aryloxy, pyridyl, imidazolyl, heteroaryl, substituted heteroaryl, heteroaryloxy, heteroarylalkyl, heteroarylalkenyl, heteroarylalkynyl, cycloalkyloxy, pyrrolidinyl, piperidinyl, morpholino, heterocycle, (heterocycle)oxy, and (heterocycle)alkyl; and preferred heteroatoms are oxygen, nitrogen, and sulfur. It is understood that where open valences exist on these substituents they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further understood that the above substitutions can be made provided that replacing the hydrogen with the substituent does not introduce unacceptable instability to the molecules of the present invention, and is otherwise chemically reasonable.

“Pharmaceutically acceptable salts” are salts that retain the desired biological activity of the parent compound and do not impart undesired toxicological effects. Pharmaceutically acceptable salt forms include various polymorphs as well as the amorphous form of the different salts derived from acid or base additions. The acid addition salts can be formed with inorganic or organic acids. Illustrative but not restrictive examples of such acids include hydrochloric, hydrobromic, sulfuric, phosphoric, citric, acetic, propionic, benzoic, napthoic, oxalic, succinic, maleic, fumaric, malic, adipic, lactic, tartaric, salicylic, methanesulfonic, 2-hydroxyethanesulfonic, toluenesulfonic, benzenesulfonic, camphorsulfonic, and ethanesulfonic acids. The pharmaceutically acceptable base addition salts can be formed with metal or organic counterions and include, but are not limited to, alkali metal salts such as sodium or potassium; alkaline earth metal salts such as magnesium or calcium; and ammonium or tetraalkyl ammonium salts, i.e., NX₄⁺ (wherein X is C₁₋₄).

“Tautomers” are compounds that can exist in one or more forms, called tautomeric forms, which can interconvert by way of a migration of one or more hydrogen atoms in the compound accompanied by a rearrangement in the position of adjacent double bonds. These tautomeric forms are in equilibrium with each other, and the position of this equilibrium will depend on the exact nature of the physical state of the compound. It is understood that where tautomeric forms are possible, the current invention relates to all possible tautomeric forms.

“Solvates” are addition complexes in which a compound of Formula I or Formula II is combined with a pharmaceutically acceptable cosolvent in some fixed proportion. Cosolvents include, but are not limited to, water, methanol, ethanol, 1-propanol, isopropanol, 1-butanol, isobutanol, tert-butanol, acetone, methyl ethyl ketone, acetonitrile, ethyl acetate, benzene, toulene, xylene(s), ethylene glycol, dichloromethane, 1,2-dichloroethane, N-methylformamide, N,N-dimethylformamide, N-methylacetamide, pyridine, dioxane, and diethyl ether. Hydrates are solvates in which the cosolvent is water. It is to be understood that the definitions of compounds in Formula I and Formula II encompass all possible hydrates and solvates, in any proportion, which possess the stated activity.

“An effective amount” is the amount effective to treat a disease by ameliorating the pathological condition or reducing the symptoms of the disease.

“Beta adrenergic receptor agonist” refers to a class of compounds that are capable of activating beta2 adrenergic receptors. Such compounds include but are not limited to albuterol (also known as salbutamol), levalbuterol, pirbuterol, formoterol, isoproterenol, salmeterol, terbutaline, metaproterenol, fenoterol, clenbuterol, bitolterol and epinephrine. Two distinct types of “beta adrenergic receptor agonist” can be identified: short-acting beta adrenergic receptor agonists (SABA) and long-acting beta adrenergic receptor agonists (LABA). SABA refers to a class of compounds that are capable of activating beta2 adrenergic receptors and that cause a prompt increase (within 3-5 minutes) in airflow. SABAs are used on an as needed basis for the prompt relief of bronchoconstriction and its accompanying acute symptoms. Examples of SABAs are albuterol, levalbuterol, pirbuterol isoproterenol, terbutaline, metaproterenol, fenoterol, clenbuterol, bitolterol and epinephrine. LABA refers to a class of compound that are capable of activating beta2 adrenergic receptors and that have a duration of bronchodilation of at least 12 hours after a single dose. LABAs are used on a daily basis for the long-term control and prevention of symptoms of the disease. Examples of LABA include formoterol and salmeterol.

“Corticosteroids” are a class of compounds with anti-inflammatory properties whose therapeutic benefit derives from interaction with intracellular glucocorticoid receptors. Such compounds include but are not limited to beclomethasone, budesonide, ciclesonide, flunisolide, fluticasone, mometasone, triamcinolone, hydrocortisone, methylprednisolone, prednisolone, and prednisone.

“Inflammation” generally refers to a localized reaction of tissue, characterized by the influx of immune cells, which occurs in reaction to injury or infection. Specifically, “pulmonary inflammation” is characterized by migration of inflammatory cells into the interstitium and the lumen of the lung, release of pro-inflammatory cytokines and chemokines, lung tissue remodeling and lung tissue apoptosis or necrosis.

“Reduced responsiveness” as used herein, refers to a state in which disease is not well-controlled by therapy. Reduced responsiveness refers to patients that do not, or do not significantly, improve the indicia of efficacy after treatment. Such patients do not significantly reduce the number of symptoms or signs of the disease, or do not significantly reduce the degree of one or more symptoms or signs of the disease after treatment. “Significantly” refer to a detectable or a measurable level of the disease management that improves the patient's well-being. Reduced responsiveness can be due to tolerance (desensitization), viral infection, bacterial infection, allergen exposure, an increase in inflammation, corticosteroid resistance leading to uncontrolled inflammation, treatment with beta adrenergic receptor antagonists (beta blockers), workplace exposure to sensitizing chemicals, environmental exposure to irritants such as tobacco smoke, sulfite sensitivity, or some unknown reason. For example, responsiveness to the treatment to achieve control of asthma can be defined according to EPR-3 guidelines (such as frequency of exacerbations, symptoms, improvement in FEV1, ER visits, nighttime awakening, frequency of rescue use of short-acting beta agonists). Specifically, for patients that are utilizing a SABA as needed for symptom control but not a LABA for disease control, reduced responsiveness refers to a failure of an inhaled SABA to increase FEV1 by greater than 200 mL and greater than or equal to 12 percent from the baseline FEV1 measure, presence of symptoms more than 2 days per week, or nighttime awakening of more than twice per month, or interference with normal activity, or the need to use SABA more than twice per week for symptom relief, or FEV1 less than 80% predicted, or an ATAQ score of greater than or equal to 1, a ACQ score of greater than or equal to 1.5 of a ACT score of greater than 16 or the presence of greater than 1 exacerbation per year as defined in EPR-3 guidelines (see attached). Specifically, for patients that are utilizing LABA for disease control and SABA for symptom control, reduced responsiveness refers to the presence of symptoms more than 2 days per week, or nighttime awakening of more than twice per month, or interference with normal activity, or the need to use SABA more than twice per week for symptom relief, or FEV1 less than 80% predicted, or an ATAQ score of greater than or equal to 1, a ACQ score of greater than or equal to 1.5 of a ACT score of greater than 16 or the presence of greater than 1 exacerbation per year as defined in EPR-3 guidelines.

“Tolerance” or “tolerant” as used herein, does not refer to patient's tolerance to adverse effects of a therapeutic agent. “Tolerance” or “tolerant” as used herein, refers to a state of reduced responsiveness to one or more beta adrenergic receptor agonists due to the desensitization of the beta adrenergic receptor following repeated (one or more) administration of beta adrenergic receptor agonists to the patient.

Reduced responsiveness to beta adrenergic receptor agonists therapy in patients with pulmonary diseases leads to a state of uncontrolled bronchoconstriction. Some of those patients gain responsiveness to the bronchorelaxant effect of beta adrenergic receptors agonists by the combination treatment of beta adrenergic receptor agonists and corticosteroids. However, in patients with reduced responsiveness to the combined treatment with beta adrenergic receptor agonists and corticosteroids, bronchoconstriction remains uncontrolled as corticosteroid treatment fails to restore the bronchorelaxant effects of beta adrenergic receptor agonists.

Reduced responsiveness can be attributed to desensitization of the beta adrenergic receptor due to repeated administration of one or more beta adrenergic receptor agonists or other events within the beta agonist receptor signaling cascade or it can be attributable to factors such as enhanced inflammation that results from disease progression, corticosteroid resistance or a variety of other events.

Although multiple factors have been identified that may limit the efficacy of beta adrenergic receptor agonists or the efficacy of the combined treatment with beta adrenergic receptor agonists and corticosteroids, the relative contribution of each of these factors in limiting the efficacy of beta adrenergic receptor agonist or the efficacy of the combined treatment in the clinic is not clear. Furthermore, many pulmonary diseases, such as asthma, COPD, and respiratory tract illness caused by respiratory syncytial virus infection such as RSV-induced wheezing, airway hyperreactivity, or bronchiolitis, are complex and heterogenous disorders involving both genetic and environmental factors. The factors that lead to the development of uncontrolled bronchoconstriction in certain patients are not known. The genetic and environmental factors that limit the efficacy of beta adrenergic receptor agonist or the efficacy of the combined treatment in some patients may also limit the activity of other bronchodilatory agents in these patients. An effective method to treat a subset of patients who have reduced responsiveness to beta adrenergic receptors agonist or reduced responsiveness to the combined treatment and have uncontrolled bronchoconstriction has not been developed. It was not known which method would work for this subset of patients when beta adrenergic receptors agonists or the combined treatment with beta adrenergic receptor agonists and corticosteroids fail to provide efficacy.

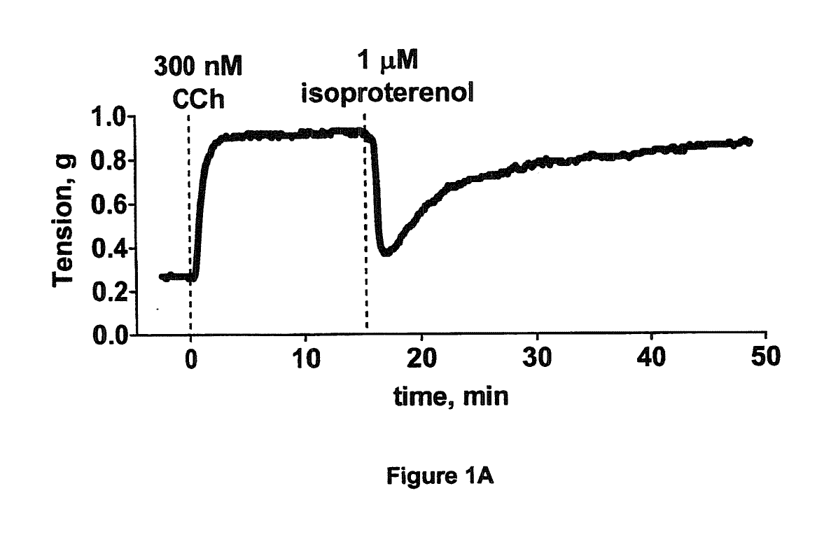

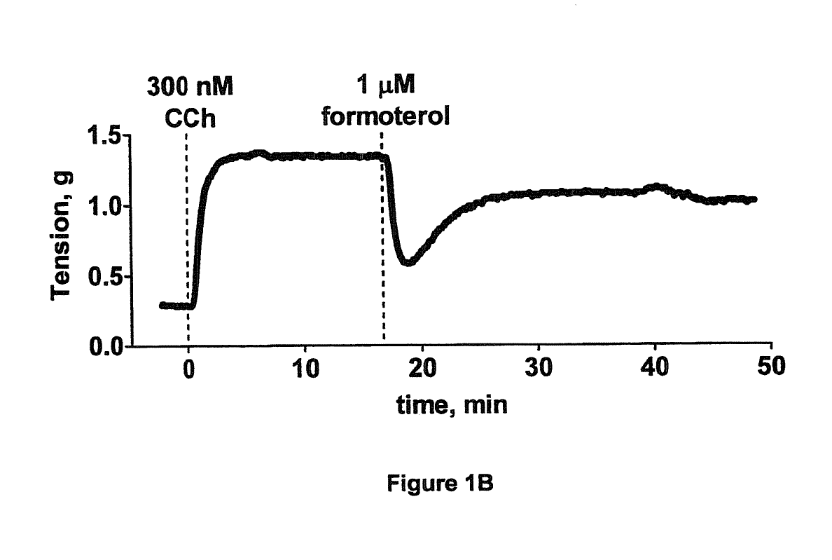

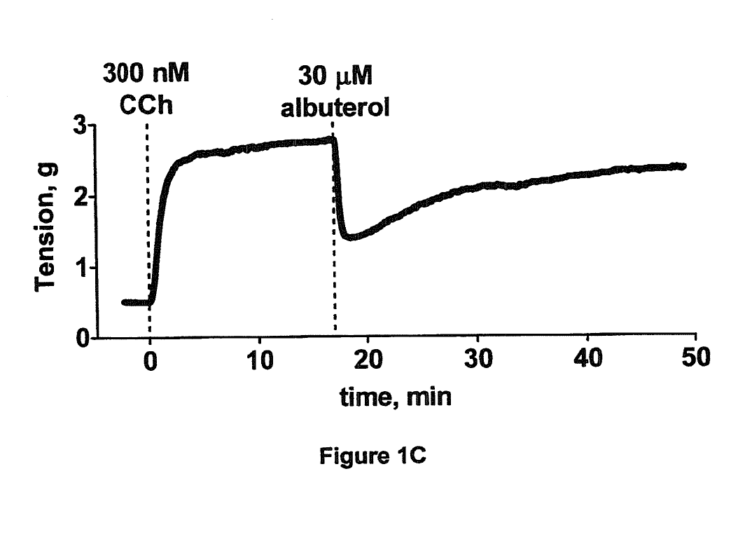

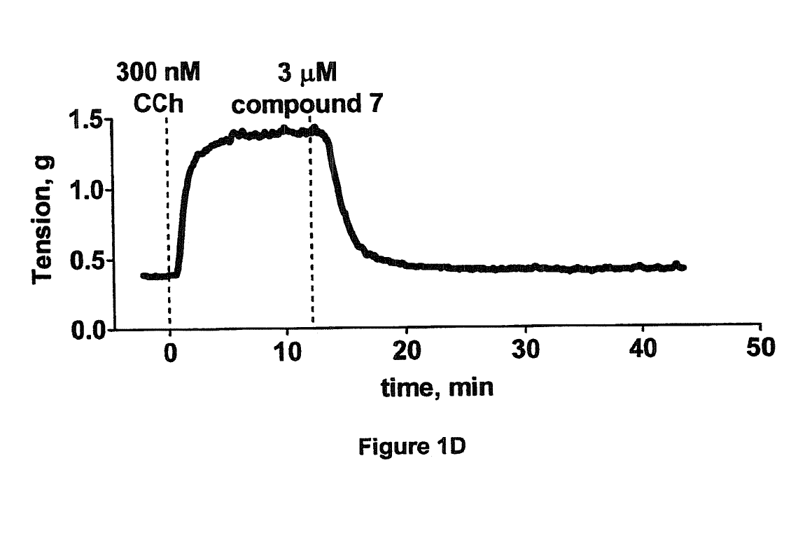

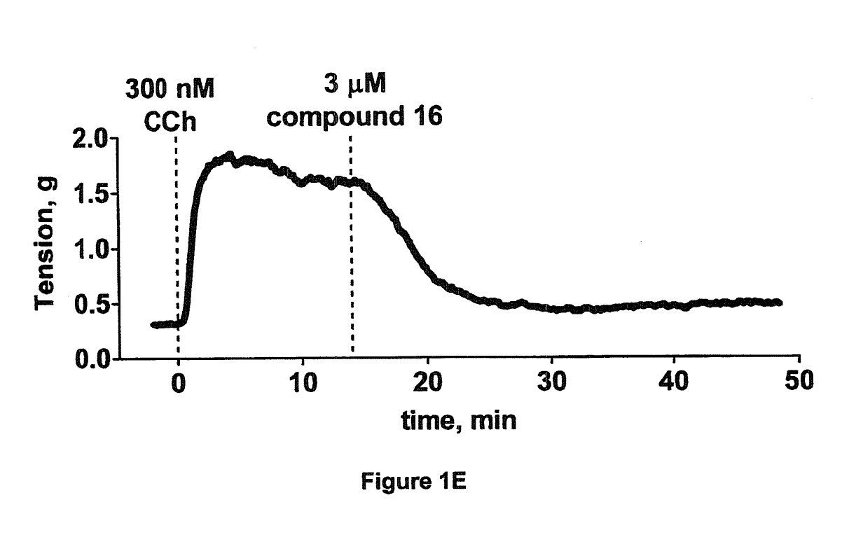

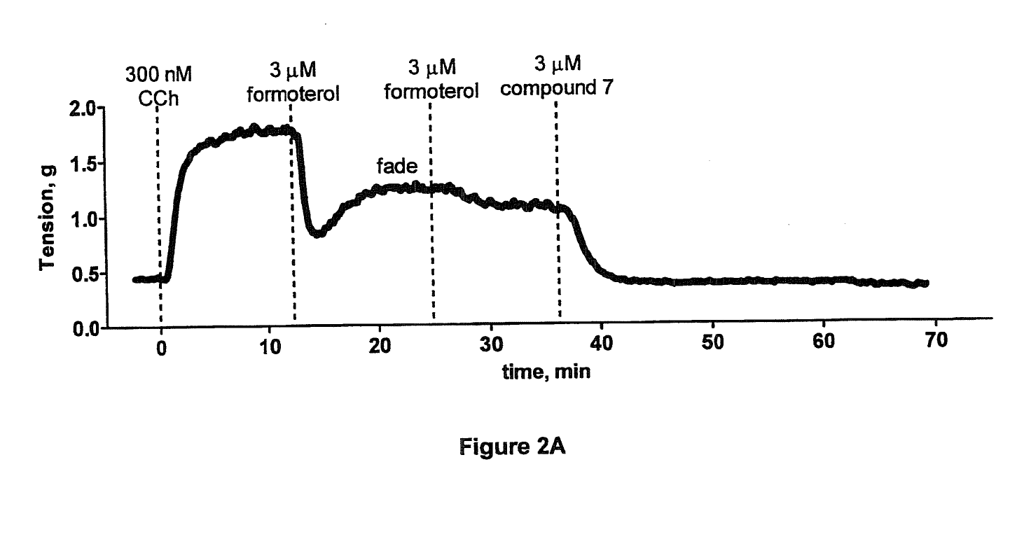

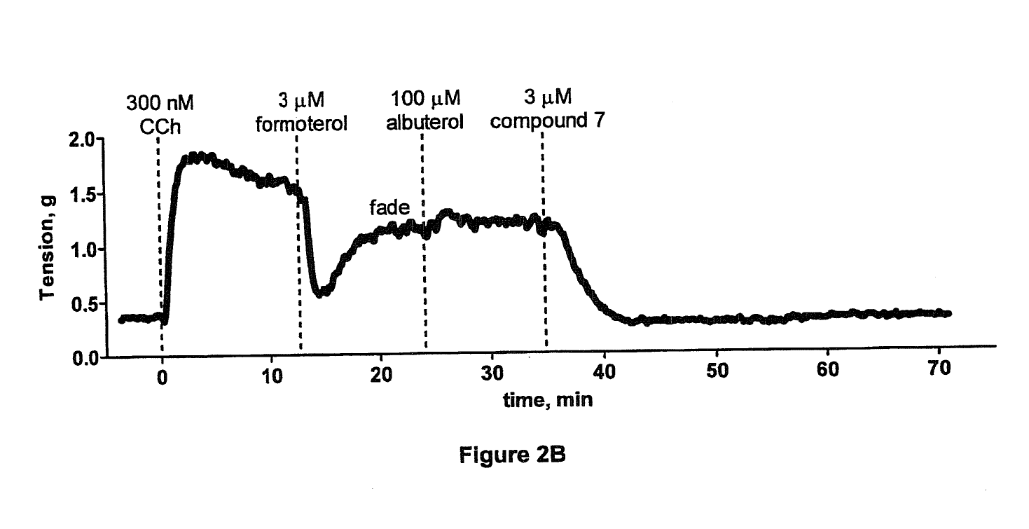

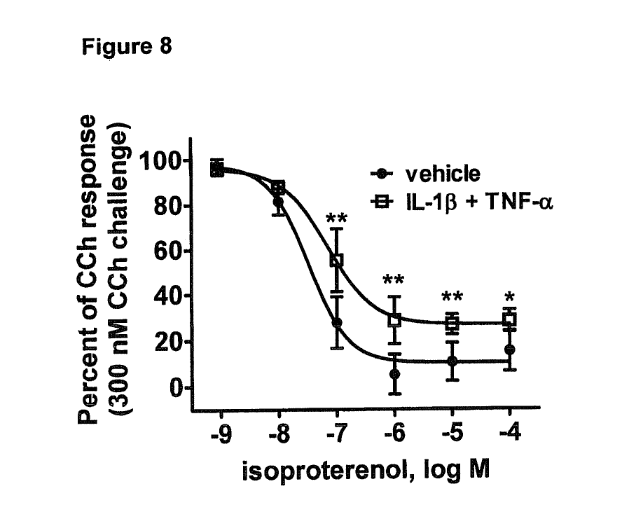

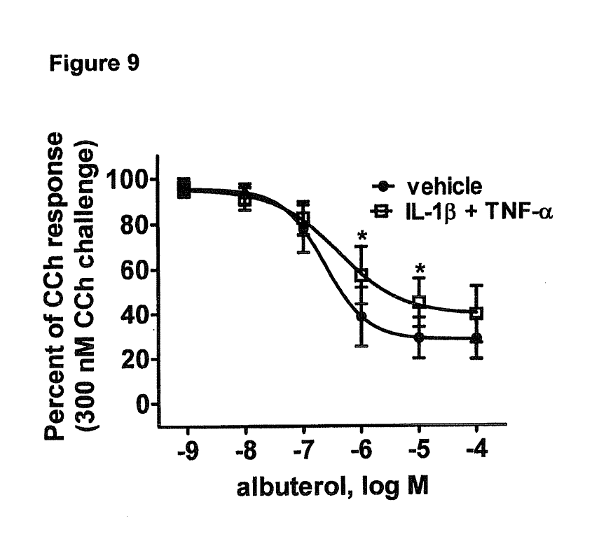

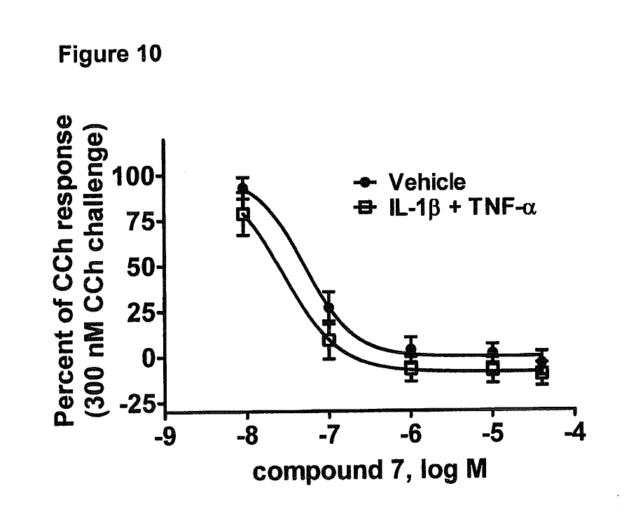

The inventors of the present invention have discovered that Rho kinase inhibitors are effective in reducing smooth muscle tone and contractibility in tissues that have reduced responsiveness upon prolonged or repeated administration of beta adrenergic receptor agonists. Goleva et al (J. Allergy Clin. Immunol. 122: 550-559, 2008) and Wanderer (Am J Respir Cell Mol Biol. 41:246-7, 2009) report that in patients with corticosteroid resistant asthma and COPD, pro-inflammatory cytokines such as TNF-α and IL-β may have increased levels. The inventors have further discovered that Rho kinase inhibitors are fully efficacious in reducing smooth muscle tone and contractibility in tissues that have been pretreated with pro-inflammatory cytokines, whereas beta adrenergic receptor agonists have reduced efficacy in tissues that have been pretreated with pro-inflammatory cytokines. For example, the inventors have discovered that application of beta adrenergic receptor agonist to carbachol-precontracted trachea resulted in an initial rapid and partial relaxation of the tissue followed by a fade of the response to a more contractile state (FIG. 1A-C). Under conditions where the response to beta adrenergic agonist had faded, application of a second dose of beta adrenergic receptor agonist (FIG. 2A) (FIG. 2B) was ineffective in relaxing the tracheal preparation (FIG. 2A, 2B). Under these conditions of reduced responsiveness to beta adrenergic receptor agonist, Rho kinase inhibitor compounds were fully efficacious in relaxing the tracheal preparation (FIG. 2A-B). Furthermore, the inventors have discovered that pretreatment of isolated tracheal preparations with pro-inflammatory cytokines, which are increased in corticosteroid resistant disease states, reduces the efficacy and potency of beta adrenergic receptor agonists (FIGS. 8, 9). However, pretreatment of isolated tracheal preparations with pro-inflammatory cytokines did not affect the efficacy and potency of Rho kinase inhibitor compounds (FIG. 10).

Although the present invention conveys this principle in airway smooth muscle, Rho kinase inhibitors can be effective in other cell types, such as inflammatory cells, in which responsiveness to beta adrenergic receptor agonists or the combined treatment with beta adrenergic receptor agonists and corticosteroids is reduced.

The invention provides a method of reducing bronchoconstriction in patients who have reduced responsiveness to treatment with beta adrenergic receptors agonists or the combined treatment with beta adrenergic receptor agonists and corticosteroids. By relaxing airway smooth muscle in patients who have no significant response to therapy with beta adrenergic agonist or the combined treatment with beta adrenergic receptor agonists and corticosteroids, the present invention provides a method of treating patients with pulmonary disease such as asthma, COPD, respiratory tract illness caused by respiratory syncytial virus infection such as RSV-induced wheezing, airway hyperreactivity, or bronchiolitis, bronchiectasis, alpha-1-antitrypsin deficiency (AATD), lymphangioleiomyomatosis (LAM), cystic fibrosis, bronchiolitis or wheezing caused by agents other than respiratory syncytial virus, chronic bronchitis, and occupational lung diseases such as coal workers' pneumoconiosis, byssinosis (brown lung disease), asbestosis and silicosis, who have reduced responsiveness to therapy with beta adrenergic receptor agonist.

The present invention provides a method for treating pulmonary diseases in patients who have reduced responsiveness to beta adrenergic receptor agonists or the combined treatment with beta adrenergic receptor agonists and corticosteroids. The method comprises the steps of: (a) identifying a patient who suffers from a pulmonary disease and has reduced responsiveness to treatment with one or more beta adrenergic receptor agonists or the combined treatment with beta adrenergic receptor agonists and corticosteroids, and (b) administering to the patient an effective amount of a Rho kinase inhibitor compound. The reduced responsiveness to beta adrenergic receptor agonist or the combined treatment with beta adrenergic receptor agonists and corticosteroids can be due to tolerance (desensitization) developed in the patient to one or more beta adrenergic receptor agonists after repeated administration of beta adrenergic receptor agonists or can be due to inflammation of the airway. The reduced responsiveness can also be due to viral infection, bacterial infection, allergen exposure, corticosteroid resistance leading to uncontrolled inflammation, treatment with beta adrenergic receptor antagonists (beta blockers), workplace exposure to sensitizing chemicals, environmental exposure to irritants such as tobacco smoke, sulfite sensitivity, or some unknown reason. The reduced responsiveness can occur upon initial treatment with a beta agonist or the combined treatment with beta adrenergic receptor agonists and corticosteroids, or upon repeated treatment with beta agonists or the combined treatment with beta adrenergic receptor agonists and corticosteroids.

In one embodiment, the patient has been treated with a corticosteroid in combination with the one or more beta adrenergic receptor agonists. In another embodiment, the patient has not been treated with a corticosteroid in combination with the one or more beta adrenergic receptor agonists. In either case, the patient has reduced responsiveness to the beta adrenergic receptor agonist treatment and requires a different treatment.

The present invention also provides a method for treating pulmonary diseases in patients who had reduced responsiveness to treatment with beta adrenergic receptor agonists but has regained responsiveness to the beta adrenergic receptor agonist after a the combined treatment with the beta adrenergic receptor agonist and a corticosteroid. The method comprises the steps of: identifying such patient and administering to the patient an effective amount of a Rho kinase inhibitor compound. In this embodiment, the administration of a single Rho kinase inhibitor compound to treat both the bronchoconstriction and inflammation is more advantageous than the combined administration of beta adrenergic receptor agonist to treat bronchoconstriction and corticosteroids to treat inflammation.

### Rho Kinase Inhibitor Compounds

Rho kinase inhibitor compounds useful for the present invention are those that inhibit serine/threonine kinase activated with the activation of Rho. Examples of Rho kinase inhibitors are compounds which inhibit ROCK-II, or ROCK-I, and other compounds that inhibit proteins having a serine/threonine kinase activity. Rho kinase inhibitors include compounds of Formula I and Formula II disclosed in WO 2008/077057-A2 and in US 2008/0214614-A1, which are incorporated herein by reference. More specifically, compounds 1 to 35 in Table 1 are examples of Rho kinase inhibitors. Additionally, Rho kinase inhibitors include (R)-trans-N-(pyridin-4-yl)-4-(1-aminoethyl)cyclohexanecarboxamide and (R)-(+)-N-(1H-pyrrolo[2,3-b]pyridin-4-yl)-4-(1-aminoethyl)-benzamide disclosed in WO 98/06433 and WO 00/09162, 1-(5-isoquinolinesulfonyl)homopiperazine and 1-(5-isoquinolinesulfonyl)-2-methylpiperazine disclosed in WO 97/23222 and Nature, 389, 990-994 (1997), (1-benzylpyrrolidin-3-yl)-(1H-indaz-ol-5-yl)amine disclosed in WO 01/56988, (1-benzylpiperidin-4-yl)-(1H-indazol-5-yl)amine disclosed in WO 02/100833, N-[2-(4-fluorophenyl)-6,7-dimethoxy-4-quinazolinyl]-N-(1H-indazol-5-yl)amine disclosed in WO 02/076976, N-4-(1H-indazol-5-yl)-6,7-dimethoxy-N-2-pyr-idin-4-yl-quinazolin-2,4-diamine disclosed in WO 02/076977, and 4-methyl-5-(2-methyl-[1,4]diazepan-1-sulfonyl)isoquinoline disclosed in WO 99/64011, 2-(4-(1H-indazol-5-yl)phenyl)propan-2-amine and other ROCK inhibitory compounds disclosed in U.S. Ser. No. 07/129,404, N-(3-(4-(1H-indazol-5-ylamino)-6-(2-(dimethylamino)ethoxy)quinazolin-2-yl)phenyl)butyramide, 2-(3-(4-(1H-indazol-5-ylamino)quinazolin-2-yl)phenoxy)-N-(pyridin-3-yl)acetamide, and other ROCK inhibitory compounds disclosed in WO 06/105081, (R)-2-amino-3-phenyl-N-(4-(pyridin-4-yl)phenyl)propanamide and other ROCK inhibitory compounds disclosed in WO 07/26920, N-(6-fluoro-1H-indazol-5-yl)-2-methyl-6-oxo-4-(4-(trifluoromethyl)phenyl)-1,4,5,6-tetrahydropyridine-3-carboxamide and other ROCK inhibitory compounds disclosed in J. Med. Chem. 2007, 50, 6-9, N-(3-(2-(4-amino-1,2,5-oxadiazol-3-yl)-1-ethyl-1H-imidazo[4,5-c]pyridin-6-yloxy)phenyl)-4-(2-morpholinoethoxy)benzamide and other ROCK inhibitory compounds disclosed in WO 05/34866, WO 05/37197, and WO 05/37198, and 1-(1-(isoquinolin-5-ylsulfonyl)piperidin-4-yl)ethanamine and other ROCK inhibitory compounds disclosed in WO 05/80394.

The Rho kinase inhibitor compounds useful for this invention include compounds of general Formula I and Formula II, and/or tautomers thereof, and/or pharmaceutically-acceptable salts, and/or solvates, and/or hydrates thereof.

A compound according to Formula I or Formula II can exist in several diastereomeric forms. The general structures of Formula I and Formula II include all diastereomeric forms of such materials, when not specified otherwise. Formula I and Formula II also include mixtures of compounds of these Formulae, including mixtures of enantiomers, diastereomers and/or other isomers in any proportion.

### A. Formula I

### Compounds of Formula I are as Follows:

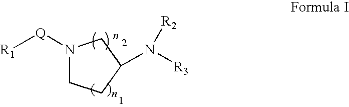

wherein: R₁ is aryl or heteroaryl, optionally substituted;
Q is C═O, SO₂, or (CR₄R₅)ₙ₃;
n₁ is 1, 2, or 3;
n₂ is 1 or 2;
n₃ is 0, 1, 2, or 3;
wherein the ring represented by

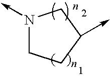

is optionally substituted by alkyl, halo, oxo, OR₆, NR₆R₇, or SR₆;
R₂ is selected from the following heteroaryl systems, optionally substituted:

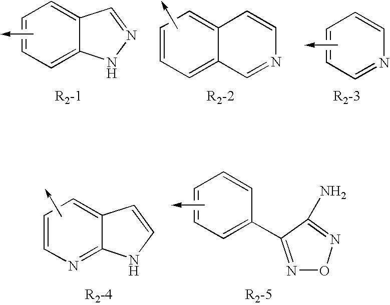

R₃-R₇ are independently H, alkyl, alkenyl, alkynyl, cycloalkyl, cycloalkenyl, cycloalkylalkyl, cycloalkylalkenyl, or cycloalkylalkynyl optionally substituted.

In Formula I, the preferred R₁ is substituted aryl, the more preferred R₁ is substituted phenyl, the preferred Q is (CR₄R₅)ₙ₃, the more preferred Q is CH₂, the preferred n₁ is 1 or 2, the preferred n₂ is 1, the preferred n₃ is 1 or 2, and the preferred R₃-R₇ are H.

[1] One embodiment of the invention is represented by Formula I, in which R₂ is 5-indazolyl or 6-indazolyl (R₂-1), optionally substituted.
[1a] In embodiment 1, R₂-1 is substituted by one or more alkyl or halo substituents.
[1b] In embodiment 1, R₂-1 is substituted by one or more amino, alkylamino, hydroxyl, or alkoxy substituents.
[1c] In embodiment 1, R₂-1 is unsubstituted.
[2] In another embodiment, the invention is represented by Formula I in which R₂ is 5-isoquinolinyl or 6-isoquinolinyl (R₂-2), optionally substituted.
[2a] In embodiment 2, R₂-2 is substituted by one or more alkyl or halo substituents.
[2b] In embodiment 2, R₂-2 is substituted by one or more amino, alkylamino, hydroxyl, or alkoxy substituents.
[2c] In embodiment 2, R₂-2 is unsubstituted.
[3] In another embodiment, the invention is represented by Formula I in which R₂ is 4-Pyridyl or 3-pyridyl (R₂-3), optionally substituted.
[3a] In embodiment 3, R₂-3 is substituted by one or more alkyl or halo substituents.
[3b] In embodiment 3, R₂-3 is substituted by one or more amino, alkylamino, hydroxyl, or alkoxy substituents.
[3c] In embodiment 3, R₂-3 is unsubstituted.
[4] In another embodiment, the invention is represented by Formula I in which R₂ is 7-azaindol-4-yl or 7-azaindol-5-yl (R₂-4), optionally substituted.
[4a] In embodiment 4, R₂-4 is substituted by one or more alkyl or halo substituents.
[4b] In embodiment 4, R₂-4 is substituted by one or more amino, alkylamino, hydroxyl, or alkoxy substituents.
[4c] In embodiment 4, R₂-4 is unsubstituted.
[5] In another embodiment, the invention is represented by Formula I in which R₂ is 4-(3-amino-1,2,5-oxadiazol-4-yl)phenyl or 3-(3-amino-1,2,5-oxadiazol-4-yl)phenyl (R₂-5), optionally substituted.
[5a] In embodiment 5, R₂-5 is unsubstituted.
[6] In another embodiment, the invention is represented by Formula I in which R₂ is one of the groups R₂-1-R₂-5, substituted by one or more alkyl, halo, amino, alkylamino, hydroxyl, or alkoxy substituents.
[6a] In embodiment 6, R₂ is substituted by one or more alkyl or halo substituents.
[6b] In embodiment 6, R₂ is substituted by one or more amino, alkylamino, hydroxyl, or alkoxy substituents.
[7] In another embodiment, the invention is represented by Formula I in which R₂ is one of the groups R₂-1-R₂-5, and is unsubstituted.
[8] In another embodiment, the invention is represented by Formula I in which R₃ is H.
[9] In another embodiment, the invention is represented by Formula I in which Q is (CR₄R₅)ₙ₃, and n₃ is 1 or 2.
[10] In another embodiment, the invention is represented by Formula I in which Q is (CH₂)ₙ₃, and n₃ is 1.
[11] In another embodiment, the invention is represented by Formula I in which R₁ is aryl or heteroaryl substituted with one or more alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, optionally further substituted.

Compounds exemplifying embodiment 11 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table I.

[12] In another embodiment, the invention is represented by Formula I in which R₁ is aryl or heteroaryl substituted with one or more heteroatom-containing substituents, with the proviso that if the R₁ substituent is acyclic and is connected to R₁ by a carbon atom, then this substituent contains at least one nitrogen or sulfur atom, with the second proviso that if the substituent is acyclic and is connected to R₁ by an oxygen or nitrogen atom, then this substituent contains at least one additional oxygen, nitrogen or sulfur atom, and with the third proviso that if the substituent is connected to R₁ by a sulfone linkage “—SO₂—”, then R₂ is not nitrogen- or oxygen-substituted R₂-2.
[12a] In embodiment 12, the heteroatom-containing substituent is connected to R₁ by an oxygen or nitrogen atom.
[12b] In embodiment 12, the heteroatom-containing substituent is connected to R₁ by a sulfide linkage, “—S—”.

Compounds exemplifying embodiment 12 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table I.

[13] In another embodiment, the invention is represented by Formula I in which R₁ is aryl or heteroaryl substituted with one or more alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, which are further substituted with one or more heteroatom-containing substituents, with the proviso that if the R₁ substituent is acyclic and its heteroatom-containing substituent falls on the carbon by which it is attached to R₁, then the heteroatom-containing substituent contains at least one nitrogen or sulfur atom.

Compounds exemplifying embodiment 13 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, and 1.122, shown below in Table I.

[14] In another embodiment, the invention is represented by Formula I in which R₁ is aryl or heteroaryl substituted with one or more alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, optionally further substituted, and R₂ is 5-indazolyl (R₂-1) or 5-isoquinolinyl (R₂-2), optionally substituted.
[14a] In embodiment 14, R₂ is 5-indazolyl (R₂-1), optionally substituted by one or more alkyl, halo, amino, alkylamino, hydroxyl, or alkoxy substituents.
[14b] In embodiment 14, R₂ is 5-isoquinolinyl (R₂-2), optionally substituted by one or more alkyl, halo, amino, alkylamino, hydroxyl, or alkoxy substituents.
[14c] In embodiment 14, R₂ is unsubstituted.

Compounds exemplifying embodiment 14 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table I.

[15] In another embodiment, the invention is represented by Formula I in which R₁ is aryl or heteroaryl substituted with one or more heteroatom-containing substituents, and R₂ is 5-indazolyl (R₂-1) or 5-isoquinolinyl (R₂-2), optionally substituted, with the proviso that if the R₁ substituent is acyclic and is connected to R₁ by a carbon atom, then this substituent contains at least one nitrogen or sulfur atom, with the second proviso that if the substituent is acyclic and is connected to R₁ by an oxygen or nitrogen atom, then this substituent contains at least one additional oxygen, nitrogen or sulfur atom, and with the third proviso that if the substituent is connected to R₁ by a sulfone linkage “—SO₂—”, then R₂ is not nitrogen- or oxygen-substituted R₂-2.
[15a] In embodiment 15, R₂ is 5-indazolyl (R₂-1), optionally substituted by one or more alkyl, halo, amino, alkylamino, hydroxyl, or alkoxy substituents.
[15b] In embodiment 15, R₂ is 5-isoquinolinyl (R₂-2), optionally substituted by one or more alkyl, halo, amino, alkylamino, hydroxyl, or alkoxy substituents.
[15c] In embodiment 15, R₂ is unsubstituted.
[15d] In embodiment 15, the heteroatom-containing substituent is connected to R₁ by an oxygen or nitrogen atom.
[15e] In embodiment 15, the heteroatom-containing substituent is connected to R₁ by a sulfide linkage, “—S—”.

Compounds exemplifying embodiment 15 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table I.

[16] In another embodiment, the invention is represented by Formula I in which R₁ is aryl or heteroaryl substituted with one or more alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, at least one of which is further substituted with one or more heteroatom-containing substituents, and R₂ is 5-indazolyl (R₂-1) or 5-isoquinolinyl (R₂-2), optionally substituted, with the proviso that if the R₁ substituent is acyclic and its heteroatom-containing substituent falls on the carbon by which it is attached to R₁, then the heteroatom-containing substituent contains at least one nitrogen or sulfur atom.
[16a] In embodiment 16, R₂ is 5-indazolyl (R₂-1), optionally substituted by one or more alkyl, halo, amino, alkylamino, hydroxyl, or alkoxy substituents.
[16b] In embodiment 16, R₂ is 5-isoquinolinyl (R₂-2), optionally substituted by one or more alkyl, halo, amino, alkylamino, hydroxyl, or alkoxy substituents.
[16c] In embodiment 16, R₂ is unsubstituted.

Compounds exemplifying embodiment 16 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, and 1.122, shown below in Table I.

### B. Formula II

A preferred compound of Formula I is where R₁═Ar—X, shown below as Formula II:

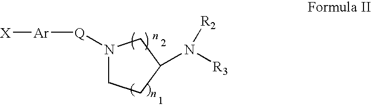

wherein:
Ar is a monocyclic or bicyclic aryl or heteroaryl ring, such as phenyl;
X is from 1 to 3 substituents on Ar, each independently in the form Y—Z, in which Z is attached to Ar;
Y is one or more substituents on Z, and each is chosen independently from H, halogen, or the heteroatom-containing substituents, including but not limited to OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀;

Each instance of Z is chosen independently from alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, heterocycle, (heterocycle)alkyl, (heterocycle)alkenyl, (heterocycle)alkynyl, or is absent; R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents, including but not limited to OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, or NR₁₁C(═O)NR₁₂R₁₃;

R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents, including but not limited to OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, or NR₁₄C(═O)NR₁₅R₁₆; any two of the groups R₈, R₉ and R₁₀ are optionally joined with a link selected from the group consisting of bond, —O—, —S—, —SO—, —SO₂—, and —NR₁₇— to form a ring;

R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle.

In Formula II, the preferred Y is H, halogen, OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀, the more preferred Y is H, halogen, OR₈, SR₈, SOR₈, SO₂R₈, SO₂NR₈R₉, NR₈SO₂R₉, CONR₈R₉, or NR₈C(═O)NR₉R₁₀, the preferred Z is alkyl, alkenyl, alkynyl, cycloalkyl, cycloalkenyl, cycloalkylalkyl, or is absent; the more preferred Z is alkyl, alkenyl, alkynyl, cycloalkyl, or is absent, the preferred Q is (CR₄R₅)ₙ₃, the more preferred Q is CH₂, the preferred n₁ is 1 or 2, the preferred n₂ is 1, the preferred n₃ is 1 or 2, the preferred R₃-R₇ are H, the preferred R₈ is H, alkyl, arylalkyl, cycloalkyl, cycloalkylalkyl, or heterocycle, the preferred R₈ substituents are halogen, OR₁₁, NR₁₁R₁₂, SR₁₁, SOR₁₁, SO₂R₁₁, SO₂NR₁₁R₁₂, NR₁₁SO₂R₁₂, CONR₁₁R₁₂, NR₁₁C(═O)R₁₂, and the preferred R₉-R₁₇ are H or alkyl.

[1] One embodiment of the invention is represented by Formula II in which R₂ is 5-indazolyl or 6-indazolyl (R₂-1), optionally substituted.
[1a] In embodiment 1, R₂-1 is substituted by one or more alkyl or halo substituents.
[1b] In embodiment 1, R₂-1 is substituted by one or more amino, alkylamino, hydroxyl, or alkoxy substituents.
[1c] In embodiment 1, R₂-1 is unsubstituted.
[2] In another embodiment, the invention is represented by Formula II in which R₂ is 5-isoquinolinyl or 6-isoquinolinyl (R₂-2), optionally substituted.
[2a] In embodiment 2, R₂-2 is substituted by one or more alkyl or halo substituents.
[2b] In embodiment 2, R₂-2 is substituted by one or more amino, alkylamino, hydroxyl, or alkoxy substituents.
[2c] In embodiment 2, R₂-2 is unsubstituted.
[3] In another embodiment, the invention is represented by Formula II in which R₂ is 4-pyridyl or 3-pyridyl (R₂-3), optionally substituted.
[3a] In embodiment 3, R₂-3 is substituted by one or more alkyl or halo substituents.
[3b] In embodiment 3, R₂-3 is substituted by one or more amino, alkylamino, hydroxyl, or alkoxy substituents.
[3c] In embodiment 3, R₂-3 is unsubstituted.
[4] In another embodiment, the invention is represented by Formula II in which R₂ is 7-azaindol-4-yl or 7-azaindol-5-yl (R₂-4), optionally substituted.
[4a] In embodiment 4, R₂-4 is substituted by one or more alkyl or halo substituents.
[4b] In embodiment 4, R₂-4 is substituted by one or more amino, alkylamino, hydroxyl, or alkoxy substituents.
[4c] In embodiment 4, R₂-4 is unsubstituted.
[5] In another embodiment, the invention is represented by Formula II in which R₂ is 4-(3-amino-1,2,5-oxadiazol-4-yl)phenyl or 3-(3-amino-1,2,5-oxadiazol-4-yl)phenyl (R₂-5), optionally substituted.
[5a] In embodiment 5, R₂-5 is unsubstituted.
[6] In another embodiment, the invention is represented by Formula II in which R₂ is one of the groups R₂-1-R₂-5, substituted by one or more alkyl, halo, amino, alkylamino, hydroxyl, or alkoxy substituents.
[6a] In embodiment 6, R₂ is substituted by one or more alkyl or halo substituents.
[6b] In embodiment 6, R₂ is substituted by one or more amino, alkylamino, hydroxyl, or alkoxy substituents.
[7] In another embodiment, the invention is represented by Formula II in which R₂ is one of the groups R₂-1-R₂-5, and is unsubstituted.
[8] In another embodiment, the invention is represented by Formula II in which R₃ is H.
[9] In another embodiment, the invention is represented by Formula II in which Q is (CR₄R₅)ₙ₃, and n₃ is 1 or 2.
[10] In another embodiment, the invention is represented by Formula II in which Q is (CH₂)ₙ₃, and n₃ is 1.
[11] In another embodiment, the invention is represented by Formula II in which Z is alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkylalkenyl, cycloalkylalkynyl, cycloalkenyl, cycloalkylalkyl, heterocycle, (heterocycle)alkyl, (heterocycle)alkenyl, or (heterocycle)alkynyl.

Compounds exemplifying embodiment 11 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table I.

[12] In another embodiment, the invention is represented by Formula II in which Z is absent, Y is a heteroatom-containing substituent, including but not limited to OR₈, NR₈R₉, SR₈, SOR₈, SO₂R₈, SO₂NR₈R₉, NR₈SO₂R₉, CONR₈R₉, NR₈C(═O)R₉, NR₈C(═O)OR₉, OC(═O)NR₈R₉, or NR₈C(═O)NR₉R₁₀, with the proviso that if the substituent Y is acyclic and is connected to Ar by a carbon atom, then this substituent contains at least one nitrogen or sulfur atom, with the second proviso, that if the substituent Y is acyclic and is connected to Ar by an oxygen or nitrogen atom, then this substituent contains at least one additional oxygen, nitrogen or sulfur atom, and with the third proviso that if the substituent Y is connected to Ar by a sulfone linkage “—SO₂—”, then R₂ is not nitrogen- or oxygen-substituted R₂-2.
[12a] In embodiment 12, the heteroatom-containing substituent is connected to R₁ by an oxygen or nitrogen atom,
[12b] In embodiment 12, the heteroatom-containing substituent is connected to R₁ by a sulfide linkage, “—S—”.

Compounds exemplifying embodiment 12 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table I.

[13] In another embodiment, the invention is represented by Formula II in which Z is alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl, and Y is a heteroatom-containing substituent, including but not limited to OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀, with the proviso that if Z is acyclic and Y falls on the carbon by which Z is attached to Ar, then Y contains at least one nitrogen or sulfur atom.

Compounds exemplifying embodiment 13 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, and 1.122, shown below in Table I.

[14] In another embodiment, the invention is represented by Formula II in which Z is alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl, and R₂ is 5-indazolyl(R₂-1) or 5-isoquinolinyl (R₂-2), optionally substituted.
[14a] In embodiment 14, R₂ is 5-indazolyl (R₂-1), optionally substituted by one or more alkyl, halo, amino, alkylamino, hydroxyl, or alkoxy substituents.
[14b] In embodiment 14, R₂ is 5-isoquinolinyl (R₂-2), optionally substituted by one or more alkyl, halo, amino, alkylamino, hydroxyl, or alkoxy substituents.
[14c] In embodiment 14, R₂ is unsubstituted.

Compounds exemplifying embodiment 14 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table I.

[15] In another embodiment, the invention is represented by Formula II in which Z is absent, Y is a heteroatom-containing substituent, including but not limited to OR₈, NR₈R₉, SR₈, SOR₈, SO₂R₈, SO₂NR₈R₉, NR₈SO₂R₉, CONR₈R₉, NR₈C(═O)R₉, NR₈C(═O)OR₉, OC(═O)NR₈R₉, or NR₈C(═O)NR₉R₁₀, and R₂ is 5-indazolyl (R₂-1) or 5-isoquinolinyl (R₂-2), optionally substituted, with the proviso that if the substituent Y is acyclic and is connected to Ar by a carbon atom, then this substituent contains at least one nitrogen or sulfur atom, with the second proviso that if the substituent Y is acyclic and is connected to Ar by an oxygen or nitrogen atom, then this substituent contains at least one additional oxygen, nitrogen or sulfur atom, and with the third proviso that if the substituent Y is connected to Ar by a sulfone linkage “—SO₂—”, then R₂ is not nitrogen- or oxygen-substituted R₂-2.
[15a] In embodiment 15, R₂ is 5-indazolyl (R₂-1), optionally substituted by one or more alkyl, halo, amino, alkylamino, hydroxyl, or alkoxy substituents.
[15b] In embodiment 15, R₂ is 5-isoquinolinyl (R₂-2), optionally substituted by one or more alkyl, halo, amino, alkylamino, hydroxyl, or alkoxy substituents.
[15c] In embodiment 15, R₂ is unsubstituted.
[15d] In embodiment 15, the heteroatom-containing substituent is connected to R₁ by an oxygen or nitrogen atom.
[15e] In embodiment 15, the heteroatom-containing substituent is connected to R₁ by a sulfide linkage, “—S—”.

Compounds exemplifying embodiment 15 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table I.

[16] In another embodiment, the invention is represented by Formula II in which Z is alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl, and Y is a heteroatom-containing substituent, including but not limited to OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀, and R₂ is 5-indazolyl (R₂-1) or 5-isoquinolinyl (R₂-2), optionally substituted, with the proviso that if Z is acyclic and Y falls on the carbon by which Z is attached to Ar, then Y contains at least one nitrogen or sulfur atom.
[16a] In embodiment 16, R₂ is 5-indazolyl(R₂-1), optionally substituted by one or more alkyl, halo, amino, alkylamino, hydroxyl, or alkoxy substituents.
[16b] In embodiment 16, R₂ is 5-isoquinolinyl (R₂-2), optionally substituted by one or more alkyl, halo, amino, alkylamino, hydroxyl, or alkoxy substituents.
[16c] In embodiment 16, R₂ is unsubstituted.
[16d] In embodiment 16, Ar is heteroaryl.

Compounds exemplifying embodiment 16 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, 1.122, and 1.123, shown below in Table A.

In Embodiments 11-16 of Formula II, the preferred Q is (CR₄R₅)ₙ₃, the more preferred Q is CH₂, the preferred n₁ is 1 or 2, the preferred n₂ is 1, the preferred n₃ is 1 or 2, and the preferred R₃ is H.

The inventors have discovered certain compounds of Formula II that have properties that render them particularly useful for treating the conditions addressed by the invention. In particular, these preferred compounds of Embodiments 14, 15 and 16 can be described as compounds of Formula II in which R₂, R₃, n₁, and n₂ are limited to the combinations shown in Formulae IIa, IIb, and IIc:

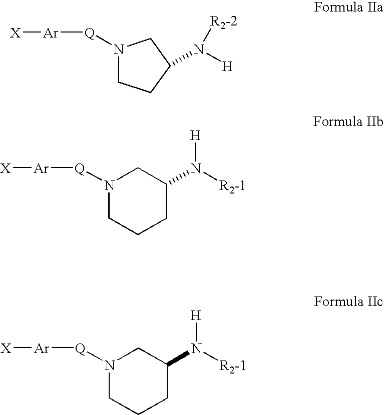

In Formulae IIa, IIb, and IIc, the stereochemistry of the central pyrrolidine or piperidine ring is limited to the R, R, and S configurations respectively, as drawn.

In Formula IIa, IIb, and IIc, Q is C═O, SO₂, or (CR₄R₅)ₙ₃; where R₄ and R₅ are independently H, alkyl, cycloalkyl, optionally substituted. The preferred R₄ and R₅ are H or unsubstituted alkyl. The preferred Q is CH₂.

In Formula IIa, IIb, and IIc, a preferred R₂ substituent is halo, alkyl, cycloalkyl, hydroxyl, alkoxy, cycloalkyloxy, amino, alkylamino, or R₂ is unsubstituted. A more preferred R₂ substituent is halo, methyl, ethyl, isopropyl, cyclopropyl, hydroxyl, methoxy, ethoxy, amino, methylamino, dimethylamino, or R₂ is unsubstituted.

In a more preferred form of Formulae IIa, IIb, and IIc, Ar is phenyl or a 6,5- or 6,6-fused bicyclic heteroaryl ring, substituted by 1 or 2 substituents X, and Q is CH₂. The most preferred 6,5-fused bicyclic heteroaryl rings are benzofuran, benzothiophene, indole, and benzimidazole.

In its more preferred form, Ar of Formulae IIa, IIb, and IIc is mono- or disubstituted when Ar is phenyl, with 3-substituted, 4-substituted, 2,3-disubstituted, and 3,4-disubstituted being most preferred. When Ar is bicyclic heteroaryl, a monosubstituted Ar is most preferred.

The inventors have found that certain members of Formulae IIa, IIb, and IIc, as defined above, are particularly useful in treating the conditions addressed in this invention. The compounds of the invention are multikinase inhibitors, with inhibitory activity against ROCK1 and ROCK2, in addition to several other kinases in individual compound cases. These kinase inhibitory properties endow the compounds of the invention not only with smooth muscle relaxant properties, but additionally with antiproliferative, antichemotactic, and cytokine secretion inhibitory properties that render them particularly useful in treating conditions with proliferative or inflammatory components as described in the invention.

[17] In particular, we have found that compounds in which R₂ is R₂-2 are particularly potent inhibitors of both ROCK1 and ROCK2, and that these agents inhibit the migration of neutrophils toward multiple chemotactic stimuli and inhibit the secretion of the cytokines IL-1β, TNF-α and IL-9 from LPS-stimulated human monocytes. Compounds in which Ar is heteroaryl, particularly 6,5-fused bicyclic heteroaryl, are especially preferred. These compounds are of particular value in addressing conditions with an inflammatory component.

Compounds exemplifying embodiment 17 include compounds 2.020, 2.021, 2.022, 2.026, 2.031, 2.033, 2.034, 2.038, 2.039, 2.040, 2.041, 2.043, 2.044, 2.054, 2.058, 2.059, 2.060, 2.063, 2.064, 2.066, 2.067, 2.068, 2.069, 2.070, 2.071, 2.072, 2.073, 2.076, 2.077, 2.078, 2.079, 2.080, 2.081, 2.082, 2.087, 2.092, 2.093, 2.094, 2.095, 2.096, 2.097, 2.098, 2.099, and 2.100.

[18] In another embodiment, we have found that compounds of Formula IIc are potent and selective inhibitors of ROCK2, with comparatively lower inhibitory potency against ROCK1. We have demonstrated that compounds of this class typically show good smooth muscle relaxation properties and that smooth muscle relaxation effects in this class are generally correlated with ROCK2 potency. Compounds in which Ar is phenyl are particularly preferred, and compounds bearing one polar group X1 in the 3-position and a second group X2 in the 4-position are most preferred. Compounds of this embodiment are of particular value in addressing conditions where relaxation of smooth muscle, in particular vascular and bronchial smooth muscle, is of highest importance.

Compounds exemplifying embodiment 18 include compounds 1.075, 1.077, 1.090, 1.091, 1.094, 1.095, 1.107, 1.109, 1.117, 1.118, 1.124, 1.152, 1.153, 1.157, 1.158, 1.165, 1.168, 1.176, 1.181, 1.182, 1.184, 1.185, 1.186, 1.187, 1.195, 1.196, 1.197, 1.198, 1.199, 1.200, 1.201, 1.213, 1.214, 1.215, 1.217, 1.218, 1.219, 1.223, 1.224, 1.228, 1.229, 1.230, 1.233, 1.234, 1.236, 1.237, 1.238, 1.239, 1.240, 1.253, 1.255, 1.261, 1.269, 1.270, 1.272, 1.274, 1.275, 1.280, and 1.282.

[19] In another embodiment, the inventors have found that compounds of Formula IIb are potent mixed inhibitors of ROCK1 and ROCK2, display additional inhibitory activity against the kinases Akt3 and p70S6K, and that these compounds generally display potent antiproliferative activity in models of smooth muscle cell proliferation. Compounds of this class are of particular value in addressing conditions in which an antiproliferative component is desired in combination with a smooth muscle relaxing effect.

Compounds exemplifying embodiment 19 include compounds 1.074, 1.076, 1.092, 1.093, 1.096, 1.097, 1.106, 1.108, 1.113, 1.115, 1.116, 1.123, 1.125, 1.126, 1.127, 1.128, 1.129, 1.139, 1.140, 1.147, 1.159, 1.160, 1.161, 1.162, 1.174, 1.188, 1.189, 1.193, 1.194, 1.202, 1.205, 1.206, 1.207, 1.208, 1.211, 1.212, 1.221, 1.222, 1.225, 1.231, 1.232, 1.235, 1.244, 1.248, 1.249, 1.258, 1.259, 1.260, 1.262, 1.263, 1.264, 1.265, 1.266, 1.267, 1.268, 1.271, 1.273, 1.276, and 1.281.

[20] In another embodiment, the inventors have found that certain compounds of Formulae IIa, IIb, and IIc distribute preferentially to the lung on oral dosing. In particular, compounds in which Ar is a lipophilic bicyclic heteroaryl group are preferred for this dosing behavior. Compounds of this type are especially useful for treating diseases of the lung by oral dosing while minimizing impact on other tissues.

Compounds exemplifying embodiment 20 include compounds 1.107, 1.109, 1.165, 1.106, 1.108, 2.058, 1.162, 1.264, 1.268, 1.271, 1.273, 1.217, 1.269, 2.059, 2.060, 2.066, and 2.072.

As discussed above for the compounds of Formulae Ia, Ib, and Ic, preparation of compounds of Formulae IIa, IIb, and IIc can be problematic using methods commonly known in the art. The inventors have disclosed and exemplified in US2008/0214614A1 methods to allow successful protection, coupling, and deprotection sequence that allows the successful preparation of the compounds of Formulae IIb and IIc and the demonstration of their useful biological properties.

The present compounds are useful for both oral and topical use, including use by the inhalation route. To be therapeutically effective in this way, the compounds must have both adequate potency and proper pharmacokinetic properties such as good permeability across the biological surface relevant to the delivery route. In general, compounds of Formulae I and II bearing polar functionality, particularly on Ar, have preferred absorption properties and are particularly suitable for topical use. In general, compounds bearing small lipophilic functional groups have good ROCK inhibitory potency.

R₁ substitution in Formula I and X in Formula II are important factors for pharmacokinetic properties and ROCK inhibitory potency. Specifically, compounds bearing polar functionality, especially those specified in the embodiments 11, 12, 13, 14, 15, and 16 in Formulae I and II, above, are particularly suitable for topical use with adequate ROCK inhibiting activity. Compounds bearing small lipophilic functional groups, as specified in the embodiments 11, 12, 13, 14, 15, and 16 in Formulae I and II, above, display ROCK inhibition with adequate permeability across biological surfaces. Compounds bearing substituents of both types are particularly preferred, and when R₁ (Formula I) or Ar (Formula II) is a phenyl ring, compounds with small lipophilic groups in the 4-position and polar functionality in the 3-position are most preferred.

Specific compounds illustrative of Formula I and Formula II are shown in the following Table A. The example compounds have been numbered in such a way that numbers of the form 1.nnn indicate compounds in which R₂ is R₂-1, numbers of the form 2.nnn indicate compounds in which R₂ is R₂-2, and so on in a similar fashion for the remaining compound numbers and groups R₂. In the following structures, hydrogens are omitted from the drawings for the sake of simplicity. Tautomers drawn represent all tautomers possible. Structures are drawn to indicate the preferred stereochemistry; where stereoisomers may be generated in these compounds, structures are taken to mean any of the possible stereoisomers alone or a mixture of stereoisomers in any ratio.

TABLE A
Exemplified Compounds.

| | | Select |
|---|---|---|
| Compound | Structure | Embodiments 1-16 |
| 1.001 | 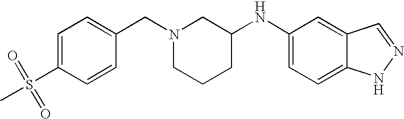 | 1c, 7, 8, 9, 10, 12, 15c |
| | N-(1-(4-(methylsulfonyl)benzyl)piperidin-3-yl)-1H- | |
| | indazol-5-amine | |
| 1.002 | 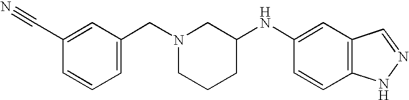 | 1c, 7, 8, 9, 10, 12, 15c |
| | 3-((3-(1H-indazol-5-ylamino)piperidin-1- | |
| | yl)methyl)benzonitrile | |
| 1.003 | 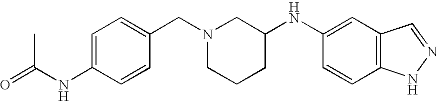 | 1c, 7, 8, 9, 10, 12a, 15c, 15d |
| | N-(4-((3-(1H-indazol-5-ylamino)piperidin-1- | |
| | yl)methyl)phenyl)acetamide | |
| 1.004 | 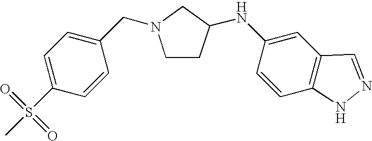 | 1c, 7, 8, 9, 10, 12, 15c |
| | N-(1-(4-(methylsulfonyl)benzyl)pyrrolidin-3-yl)-1H- | |
| | indazol-5-amine | |
| 1.005 | 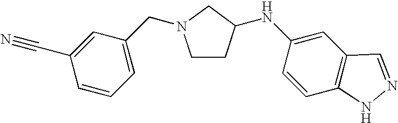 | 1c, 7, 8, 9, 10, 12, 15c |
| | 3-((3-(1H-indazol-5-ylamino)pyrrolidin-1- | |
| | yl)methyl)benzonitrile | |
| 1.006 | 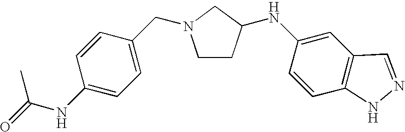 | 1c, 7, 8, 9, 10, 12a, 15c, 15d |
| | N-(4-((3-(1H-indazol-5-ylamino)pyrrolidin-1- | |
| | yl)methyl)phenyl)acetamide | |
| 1.007 | 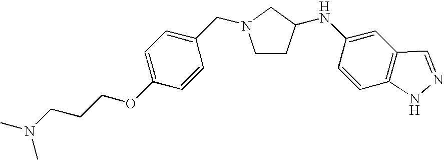 | 1c, 7, 8, 9, 10, 12a, 15c, 15d |
| | N-(1-(4-(3-(dimethylamino)propoxy)benzyl)pyrrolidin- | |
| | 3-yl)-1H-indazol-5-amine | |
| 1.008 | 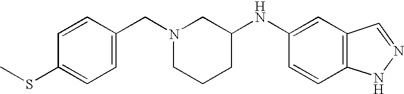 | 1c, 7,8, 9,10, 12b, 15c, 15e |
| | N-(1-(4-(methylthio)benzyl)piperidin-3-yl)-1H-indazol- | |
| | 5-amine | |
| 1.009 | 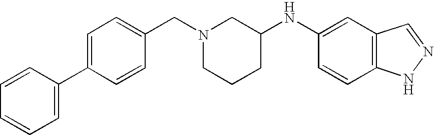 | 1c, 7, 8, 9, 10, 11, 14c |
| | N-(1-(biphenyl-4-ylmethyl)piperidin-3-yl)-1H-indazol-5- | |
| | amine | |
| 1.010 | 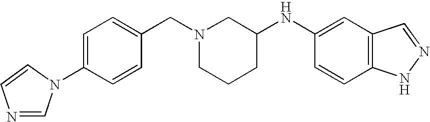 | 1c, 7, 8, 9, 10, 11, 14c |
| | N-(1-(1H-imidazol-1-yl)benzyl)piperidin-3-yl)-1H- | |
| | indazol-5-amine | |
| 1.011 | 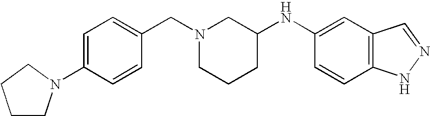 | 1c, 7, 8, 9, 10, 11, 14c |
| | N-(1-(4-(pyrrolidin-1-yl)benzyl)piperidin-3-yl)-1H- | |
| | indazol-5-amine | |
| 1.012 | 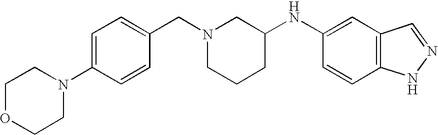 | 1c, 7, 8, 9, 10, 11, 14c |
| | N-(1-(4-morpholinobenzyl)piperidin-3-yl)-1H-indazol-5- | |
| | amine | |
| 1.013 | 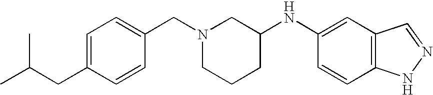 | 1c, 7, 8, 9, 10 |
| | N-(1-(4-isobutylbenzyl)piperidin-3-yl)-1H-indazol-5- | |
| | amine | |
| 1.014 | 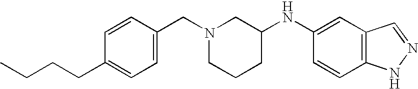 | 1c, 7, 8, 9, 10 |
| | N-(1-(4-butylbenzyl)piperidin-3-yl)-1H-indazol-5-amine | |
| 1.015 | 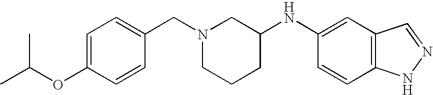 | 1c, 7, 8, 9, 10 |
| | N-(1-(4-isopropoxybenzyl)piperidin-3-yl)-1H-indazol-5- | |
| | amine | |
| 1.016 | 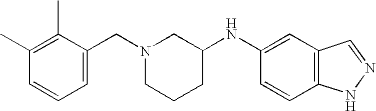 | 1c, 7, 8, 9, 10 |
| | N-(1-(2,3-dimethylbenzyl)piperidin-3-yl)-1H-indazol-5- | |
| | amine | |
| 1.017 | 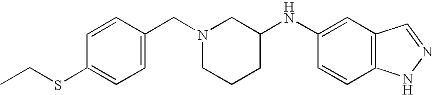 | 1c, 7, 8, 9, 10, 12b, 15c, 15e |
| | N-(1-(4-(ethylthio)benzyl)piperidin-3-yl)-1H-indazol-5- | |
| | amine | |
| 1.018 | 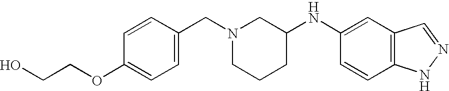 | 1c, 7, 8, 9, 10, 12a, 15c, 15d |
| | 2-(4-((3-(1H-indazol-5-ylamino)piperidin-1-yl)methyl) | |
| | phenoxy)ethanol | |
| 1.019 | 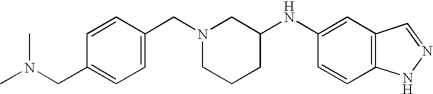 | 1c, 7, 8, 9, 10, 13, 16c |
| | N-(1-(4-((dimethylamino)methyl)benzyl)piperidin-3-yl)- | |
| | 1H-indazol-5-amine | |
| 1.020 | 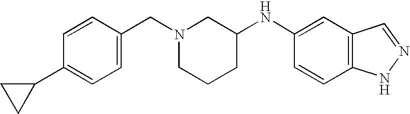 | 1c, 7, 8, 9, 10, 11, 14c |
| | N-(1-(4-cyclopropylbenzyl)piperidin-3-yl)-1H-indazol-5- | |
| | amine | |
| 1.021 | 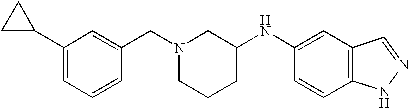 | 1c, 7, 8, 9, 10, 11, 14c |
| | N-(1-(3-cyclopropylbenzyl)piperidin-3-yl)-1H-indazol-5- | |
| | amine | |
| 1.022 | 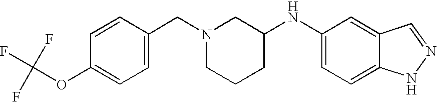 | 1c, 7, 8, 9, 10 |
| | N-(1-(4-(trifluoromethoxy)benzyl)piperidin-3-yl)-1H- | |
| | indazol-5-amine | |
| 1.023 | 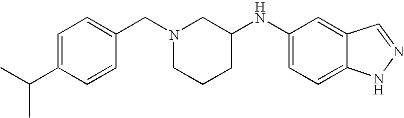 | 1c, 7, 8, 9, 10 |
| | N-(1-(4-isopropylbenzyl)piperidin-3-yl)-1H-indazol-5- | |
| | amine | |
| 1.024 | 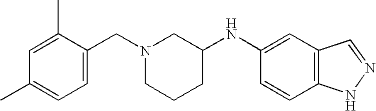 | 1c, 7, 8, 9, 10 |
| | N-(1-(2,4-dimethylbenzyl)piperidin-3-yl)-1H-indazol-5- | |
| | amine | |
| 1.025 | 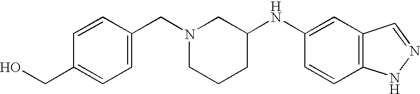 | 1c, 7, 8, 9, 10 |
| | (4-((3-(1H-indazol-5-ylamino)piperidin-1- | |
| | yl)methyl)phenyl)methanol | |
| 1.026 | 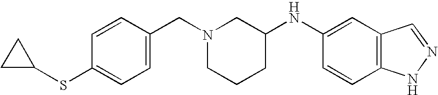 | 1c, 7, 8, 9, 10, 12b, 15c, 15e |
| | N-(1-(4-(cyclopropylthio)benzyl)piperidin-3-yl)-1H- | |
| | indazol-5-amine | |
| 1.027 | 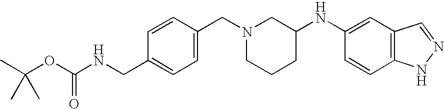 | 1c, 7, 8, 9, 10, 13, 16c |
| | tert-butyl 4-((3-(1H-indazol-5-ylamino)piperidin-1- | |
| | yl)methyl)benzylcarbamate | |
| 1.028 | 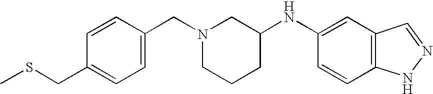 | 1c, 7, 8, 9, 10, 13, 16c |
| | N-(1-(4-(methylthiomethyl)benzyl)piperidin-3-yl)-1H- | |
| | indazol-5-amine | |
| 1.029 | 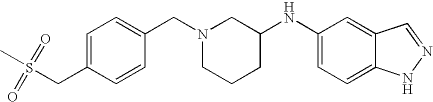 | 1c, 7, 8, 9, 10, 13, 16c |
| | N-(1-(4-(methylsulfonylmethyl)benzyl)piperidin-3-yl)- | |
| | 1H-indazol-5-amine | |
| 1.030 | 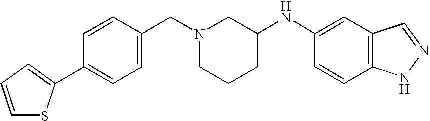 | 1c, 7, 8, 9, 10, 11, 14c |
| | N-(1-(4-(thiophen-2-yl)benzyl)piperidin-3-yl)-1H- | |
| | indazol-5-amine | |
| 1.031 | 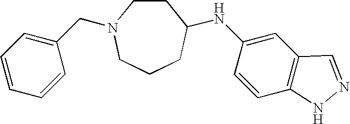 | 1c, 7, 8, 9, 10 |
| | N-(1-benzylazepan-4-yl)-1H-indazol-5-amine | |
| 1.032 | 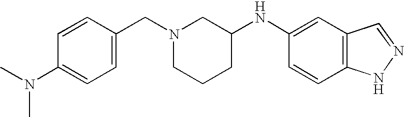 | 1c, 7, 8, 9, 10 |
| | N-(1-(4-(dimethylamino)benzyl)piperidin-3-yl)-1H- | |
| | indazol-5-amine | |
| 1.033 | 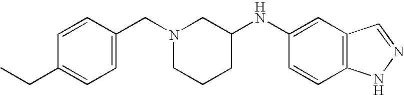 | 1c, 7, 8, 9, 10 |
| | N-(1-(4-ethylbenzyl)piperidin-3-yl)-1H-indazol-5-amine | |
| 1.034 | 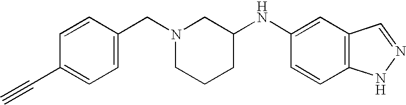 | 1c, 7, 8, 9, 10, 11, 14c |
| | N-(1-(4-ethynylbenzyl)piperidin-3-yl)-1H-indazol-5- | |
| | amine | |
| 1.035 | 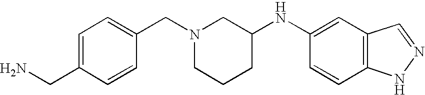 | 1c, 7, 8, 9, 10, 13 16c |
| | N-(1-(4-(aminomethyl)benzyl)piperidin-3-yl)-1H- | |
| | indazol-5-amine | |
| 1.036 | 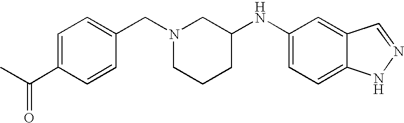 | 1c, 7, 8, 9, 10 |
| | 1-(4-((3-(1H-indazol-5-ylamino)piperidin-1- | |
| | yl)methyl)phenyl)ethanone | |
| 1.037 | 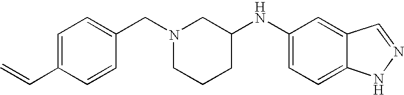 | 1c, 7, 8, 9, 10, 11, 14c |
| | N-(1-(4-vinylbenzyl)piperidin-3-yl)-1H-indazol-5-amine | |
| 1.038 | 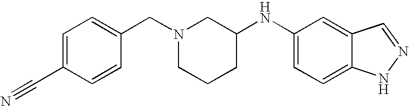 | 1c, 7, 8, 9, 10, 12, 15c |
| | 4-((3-(1H-indazol-5-ylamino)piperidin-1- | |
| | yl)methyl)benzonitrile | |
| 1.039 | 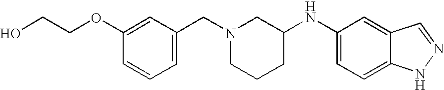 | 1c, 7, 8, 9, 10, 12a, 15c, 15d |
| | 2-(3-((3-(1H-indazol-5-ylamino)piperidin-1-yl)methyl) | |
| | phenoxy)ethanol | |
| 1.040 | 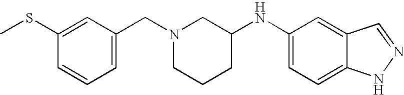 | 1c, 7, 8, 9, 10, 12b, 15c, 15e |
| | N-(1-(3-(methylthio)benzyl)piperidin-3-yl)-1H-indazol- | |
| | 5-amine | |
| 1.041 | 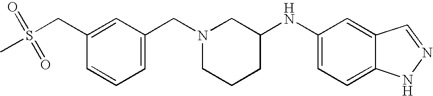 | 1c, 7, 8, 9, 10, 13, 16c |
| | N-(1-(3-(methylsulfonylmethyl)benzyl)piperidin-3-yl)- | |
| | 1H-indazol-5-amine | |
| 1.042 | 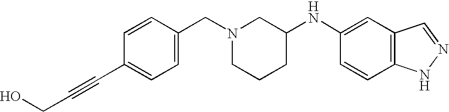 | 1c, 7, 8, 9, 10, 13, 16c |
| | 3-(4-((3-(1H-indazol-5-ylamino)piperidin-1- | |
| | yl)methyl)phenyl)prop-2-yn-1-ol | |
| 1.043 | 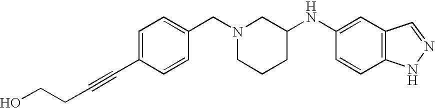 | 1c, 7, 8, 9, 10, 13, 16c |
| | 4-(4-((3-(1H-indazol-5-ylamino)piperidin-1- | |
| | yl)methyl)phenyl)but-3-yn-1-ol | |
| 1.044 | 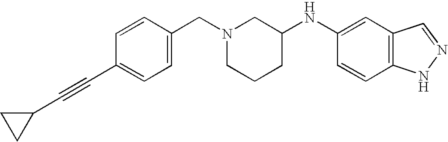 | 1c, 7, 8, 9, 10, 11, 14c |
| | N-(1-(4-(cyclopropylethynyl)benzyl)piperidin-3-yl)-1H- | |
| | indazol-5-amine | |
| 1.045 | 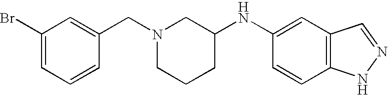 | 1c, 7, 8, 9, 10 |
| | N-(1-(3-bromobenzyl)piperidin-3-yl)-1H-indazol-5- | |
| | amine | |
| 1.046 | 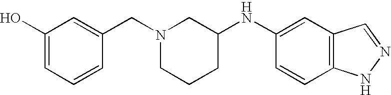 | 1c, 7, 8, 9, 10 |
| | 3-((3-(1H-indazol-5-ylamino)piperidin-1- | |
| | yl)methyl)phenol | |
| 1.047 | 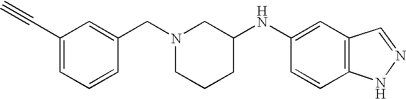 | 1c, 7, 8, 9, 10, 11, 14c |
| | N-(1-(3-ethynylbenzyl)piperidin-3-yl)-1H-indazol-5- | |
| | amine | |
| 1.048 | 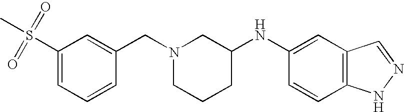 | 1c, 7, 8, 9, 10, 12, 15c |
| | N-(1-(3-(methylsulfonyl)benzyl)piperidin-3-yl)-1H- | |
| | indazol-5-amine | |
| 1.049 | 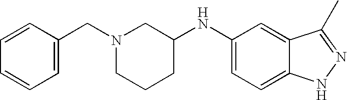 | 1a, 6a, 8, 9, 10 |
| | N-(1-benzylpiperidin-3-yl)-3-methyl-1H-indazol-5- | |
| | amine | |
| 1.050 | 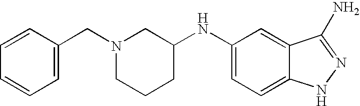 | 1b, 6b, 8, 9, 10 |
| | N5-(1-benzylpiperidin-3-yl)-1H-indazole-3,5-diamine | |
| 1.051 | 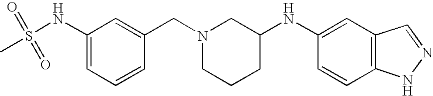 | 1c, 7, 8, 9, 10, 12a, 15c, 15d |
| | N-(3-((3-(1H-indazol-5-ylamino)piperidin-1- | |
| | yl)methyl)phenyl)methanesulfonamide | |
| 1.052 | 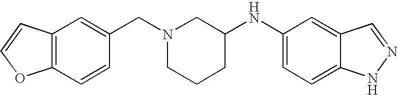 | 1c, 7, 8, 9, 10 |
| | N-(1-(benzofuran-5-ylmethyl)piperidin-3-yl)-1H- | |
| | indazol-5-amine | |
| 1.053 | 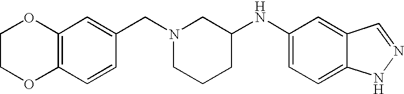 | 1c, 7, 8, 9, 10 |
| | N-(1-((2,3-dihydrobenzo[b][1,4]dioxin-6- | |
| | yl)methyl)piperidin-3-yl)-1H-indazol-5-amine | |
| 1.054 | 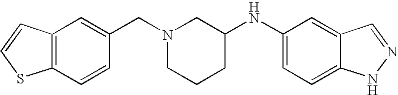 | 1c, 7, 8, 9, 10 |
| | N-(1-(benzo[b]thiophen-5-ylmethyl)piperidin-3-yl)-1H- | |
| | indazol-5-amine | |
| 1.055 | 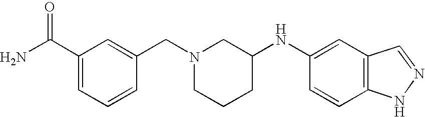 | 1c, 7, 8, 9, 10, 12, 15c |
| | 3-((3-(1H-indazol-5-ylamino)piperidin-1- | |
| | yl)methyl)benzamide | |
| 1.056 | 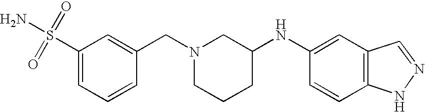 | 1c, 7, 8, 9, 10, 12, 15c |
| | 3-((3-(1H-indazol-5-ylamino)piperidin-1- | |
| | yl)methyl)benzenesulfonamide | |
| 1.057 | 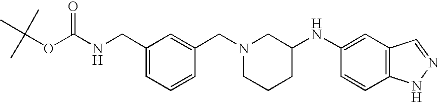 | 1c, 7, 8, 9, 10, 13, 16c |
| | tert-butyl 3-((3-(1H-indazol-5-ylamino)piperidin-1- | |
| | yl)methyl)benzylcarbamate | |
| 1.058 | 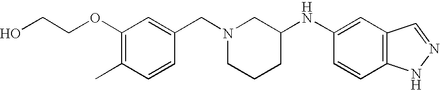 | 1c, 7, 8, 9, 10, 12a, 15c, 15d |
| | 2-(5-((3-(1H-indazol-5-ylamino)piperidin-1-yl)methyl)- | |
| | 2-methylphenoxy)ethanol | |
| 1.059 | 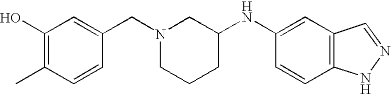 | 1c, 7, 8, 9, 10 |
| | 5-((3-(1H-indazol-5-ylamino)piperidin-1-yl)methyl)-2- | |
| | methylphenol | |
| 1.060 | 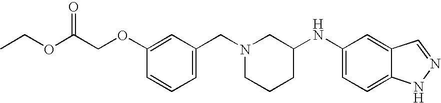 | 1c, 7, 8, 9, 10, 12a, 15c, 15d |
| | ethyl 2-(3-((3-(1H-indazol-5-ylamino)piperidin-1- | |
| | yl)methyl)phenoxy)acetate | |
| 1.061 | 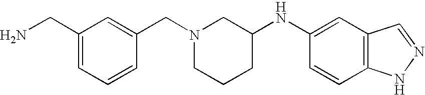 | 1c, 7, 8, 9, 10, 13, 16c |
| | N-(1-(3-(aminomethyl)benzyl)piperidin-3-yl)-1H- | |
| | indazol-5-amine | |
| 1.062 | 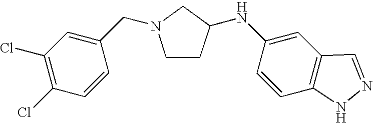 | 1c, 7, 8, 9, 10 |
| | N-(1-(3,4-dichlorobenzyl)pyrrolidin-3-yl)-1H-indazol-5- | |
| | amine | |
| 1.063 | 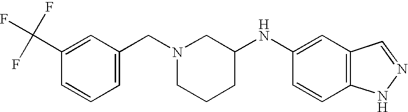 | 1c, 7, 8, 9, 10 |
| | N-(1-(3-(trifluoromethyl)benzyl)piperidin-3-yl)-1H- | |
| | indazol-5-amine | |
| 1.064 | 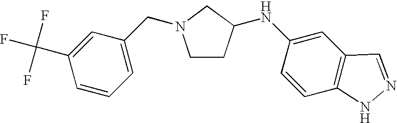 | 1c, 7, 8, 9, 10 |
| | N-(1-(3-(trifluoromethyl)benzyl)pyrrolidin-3-yl)-1H- | |
| | indazol-5-amine | |
| 1.065 | 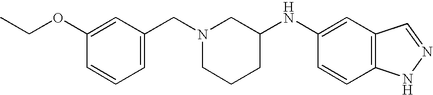 | 1c, 7, 8, 9, 10 |
| | N-(1-(3-ethoxybenzyl)piperidin-3-yl)-1H-indazol-5- | |
| | amine | |
| 1.066 | 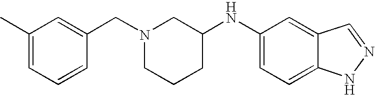 | 1c, 7, 8, 9, 10 |
| | N-(1-(3-methylbenzyl)piperidin-3-yl)-1H-indazol-5- | |
| | amine | |
| 1.067 | 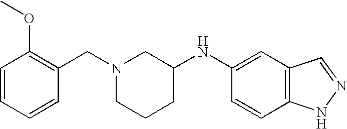 | 1c, 7, 8, 9, 10 |
| | N-(1-(2-methoxybenzyl)piperidin-3-yl)-1H-indazol-5- | |
| | amine | |
| 1.068 | 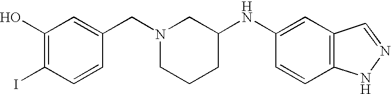 | 1c, 7, 8, 9, 10 |
| | 5-((3-(1H-indazol-5-ylamino)piperidin-1-yl)methyl)-2- | |
| | iodophenol | |
| 1.069 | 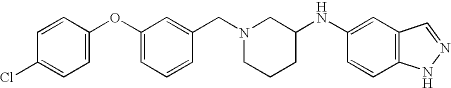 | 1c, 7, 8, 9, 10 |
| | N-(1-(3-(4-chlorophenoxy)benzyl)piperidin-3-yl)-1H- | |
| | indazol-5-amine | |
| 1.070 | 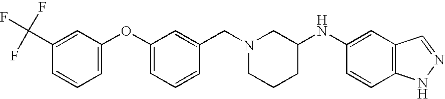 | 1c, 7, 8, 9, 10 |
| | N-(1-(3-(3-(trifluoromethyl)phenoxy)benzyl)piperidin-3- | |
| | yl)-1H-indazol-5-amine | |
| 1.071 | 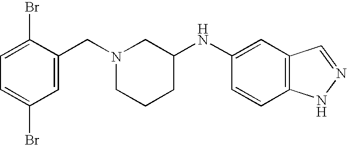 | 1c, 7, 8, 9, 10 |
| | N-(1-(2,5-dibromobenzyl)piperidin-3-yl)-1H-indazol-5- | |
| | amine | |
| 1.072 | 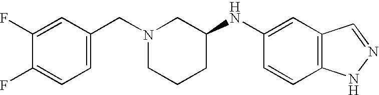 | 1c, 7, 8, 9, 10 |
| | (S)-N-(1-(3,4-difluorobenzyl)piperidin-3-yl)-1H-indazol- | |
| | 5-amine | |
| 1.073 | 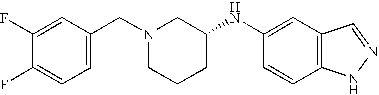 | 1c, 7, 8, 9, 10 |
| | (R)-N-(1-(3,4-difluorobenzyl)piperidin-3-yl)-1H-indazol- | |
| | 5-amine | |
| 1.074 | 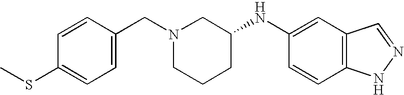 | 1c, 7, 8, 9, 10, 12b, 15c, 15e |
| | (R)-N-(1-(4-(methylthio)benzyl)piperidin-3-yl)-1H- | |
| | indazol-5-amine | |
| 1.075 | 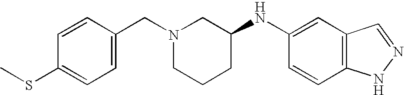 | 1c, 7, 8, 9, 10, 12b, 15c, 15e |
| | (S)-N-(1-(4-(methylthio)benzyl)piperidin-3-yl)-1H- | |
| | indazol-5-amine | |
| 1.076 | 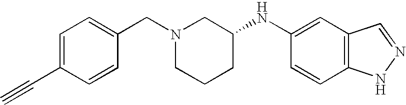 | 1c, 7, 8, 9, 10, 11, 14c |
| | (R)-N-(1-(4-ethynylbenzyl)piperidin-3-yl)-1H-indazol-5- | |
| | amine | |
| 1.077 | 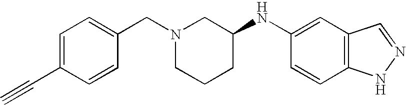 | 1c, 7, 8, 9, 10, 11, 14c |
| | (S)-N-(1-(4-ethynylbenzyl)piperidin-3-yl)-1H-indazol-5- | |
| | amine | |
| 1.078 | 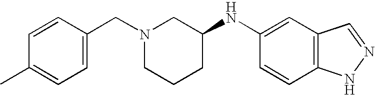 | 1c, 7, 8, 9, 10 |
| | (S)-N-(1-(4-methylbenzyl)piperidin-3-yl)-1H-indazol-5- | |
| | amine | |
| 1.079 | 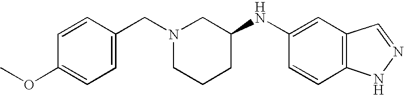 | 1c, 7, 8, 9, 10 |
| | (S)-N-(1-(4-methoxybenzyl)piperidin-3-yl)-1H-indazol- | |
| | 5-amine | |
| 1.080 | 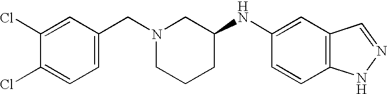 | 1c, 7, 8, 9, 10 |
| | (S)-N-(1-(3,4-dichlorobenzyl)piperidin-3-yl)-1H- | |
| | indazol-5-amine | |
| 1.082 | 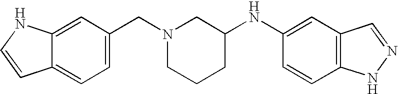 | 1c, 7, 8, 9, 10 |
| | N-(1-((1H-indol-6-yl)methyl)piperidin-3-yl)-1H-indazol- | |
| | 5-amine | |
| 1.083 | 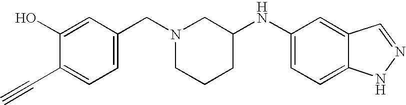 | 1c, 7, 8, 9, 10, 11, 14c |
| | 5-((3-(1H-indazol-5-ylamino)piperidin-1-yl)methyl)-2- | |
| | ethynylphenol | |
| 1.084 | 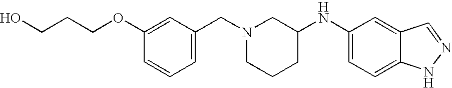 | 1c, 7, 8, 9, 10, 12a, 15c, 15d |
| | 3-(3-((3-(1H-indazol-5-ylamino)piperidin-1-yl)methyl) | |
| | phenoxy)propan-1-ol | |
| 1.085 | 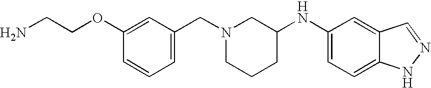 | 1c, 7, 8, 9, 10, 12a, 15c, 15d |
| | N-(1-(3-(2-aminoethoxy)benzyl)piperidin-3-yl)-1H- | |
| | indazol-5-amine | |
| 1.086 | 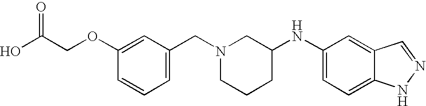 | 1c, 7, 8, 9, 10, 12a, 15c, 15d |
| | 2-(3-((3-(1H-indazol-5-ylamino)piperidin-1-yl)methyl) | |
| | phenoxy)acetic acid | |
| 1.087 | 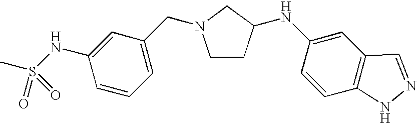 | 1c, 7, 8, 9, 10, 12a, 15c, 15d |
| | N-(3-((3-(1H-indazol-5-ylamino)pyrrolidin-1- | |
| | yl)methyl)phenyl)methanesulfonamide | |
| 1.088 | 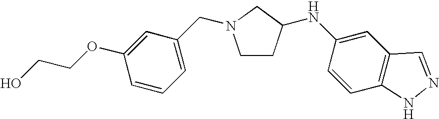 | 1c, 7, 8, 9, 10, 12a, 15c, 15d |
| | 2-(3-((3-(1H-indazol-5-ylamino)pyrrolidin-1-yl)methyl) | |
| | phenoxy)ethanol | |
| 1.089 | 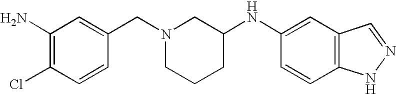 | 1c, 7, 8, 9, 10 |
| | N-(1-(3-amino-4-chlorobenzyl)piperidin-3-yl)-1H- | |
| | indazol-5-amine | |
| 1.090 | 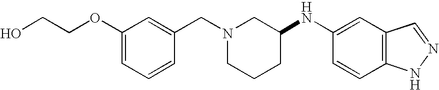 | 1c, 7, 8, 9, 10, 12a, 15c, 15d |
| | (S)-2-(3-((3-(1H-indazol-5-ylamino)piperidin-1- | |
| | yl)methyl)phenoxy)ethanol | |
| 1.091 | 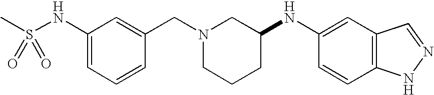 | 1c, 7, 8, 9, 10, 12a, 15c, 15d |
| | (S)-N-(3-((3-(1H-indazol-5-ylamino)piperidin-1- | |
| | yl)methyl)phenyl)methanesulfonamide | |
| 1.092 | 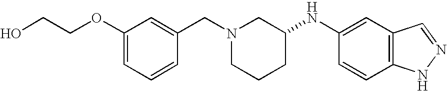 | 1c, 7, 8, 9, 10, 12a, 15c, 15d |
| | (R)-2-(3-((3-(1H-indazol-5-ylamino)piperidin-1- | |
| | yl)methyl)phenoxy)ethanol | |
| 1.093 | 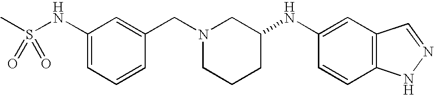 | 1c, 7, 8, 9, 10, 12a, 15c, 15d |
| | (R)-N-(3-((3-(1H-indazol-5-ylamino)piperidin-1- | |
| | yl)methyl)phenyl)methanesulfonamide | |
| 1.094 | 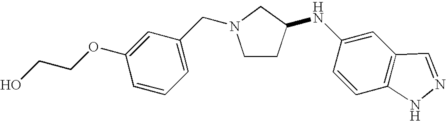 | 1c, 7, 8, 9, 10, 12a, 15c, 15d |
| | (S)-2-(3-((3-(1H-indazol-5-ylamino)pyrrolidin-1- | |
| | yl)methyl)phenoxy)ethanol | |
| 1.095 | 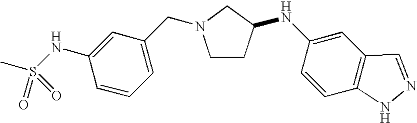 | 1c, 7, 8, 9, 10, 12a, 15c, 15d |
| | (S)-N-(3-((3-(1H-indazol-5-ylamino)pyrrolidin-1- | |
| | yl)methyl)phenyl)methanesulfonamide | |
| 1.096 | 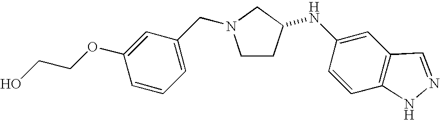 | 1c, 7, 8, 9, 10, 12a, 15c, 15d |
| | (R)-2-(3-((3-(1H-indazol-5-ylamino)pyrrolidin-1- | |
| | yl)methyl)phenoxy)ethanol | |
| 1.097 | 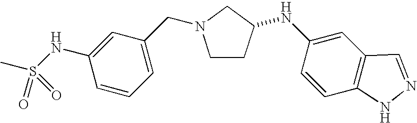 | 1c, 7, 8, 9, 10, 12a, 15c, 15d |
| | (R)-N-(3-((3-(1H-indazol-5-ylamino)pyrrolidin-1- | |
| | yl)methyl)phenyl)methanesulfonamide | |
| 1.098 | 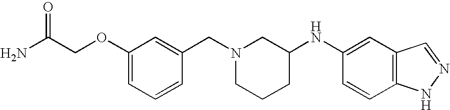 | 1c, 7, 8, 9, 10, 12a, 15c, 15d |
| | 2-(3-((3-(1H-indazol-5-ylamino)piperidin-1- | |
| | yl)methyl)phenoxy)acetamide | |
| 1.099 | 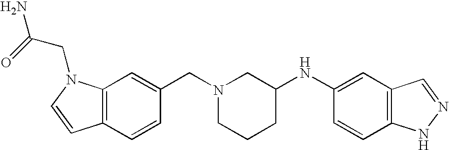 | 1c, 7, 8, 9, 10, 13, 16c |
| | 2-(6-((3-(1H-indazol-5-ylamino)piperidin-1-yl)methyl)- | |
| | 1H-indol-1-yl)acetamide | |
| 1.100 | 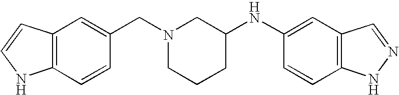 | 1c, 7, 8, 9, 10, 13, 16c |
| | N-(1-((1H-indol-5-yl)methyl)piperidin-3-yl)-1H-indazol- | |
| | 5-amine | |
| 1.101 | 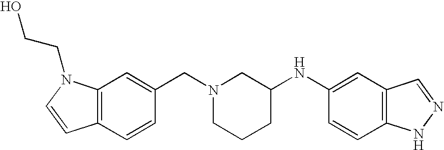 | 1c, 7, 8, 9, 10, 13, 16c |
| | 2-(6-((3-(1H-indazol-5-ylamino)piperidin-1-yl)methyl)- | |
| | 1H-indol-1-yl)ethanol | |
| 1.102 | 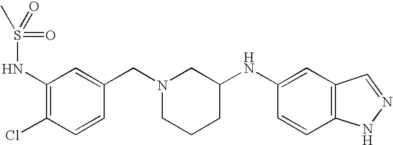 | 1c, 7, 8, 9, 10, 12a, 15c, 15d |
| | N-(5-((3-(1H-indazol-5-ylamino)piperidin-1-yl)methyl)- | |
| | 2-chlorophenyl)methanesulfonamide | |
| 1.103 | 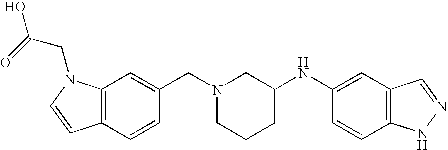 | 1c, 7, 8, 9, 10, 13, 16c |
| | 2-(6-((3-(1H-indazol-5-ylamino)piperidin-1-yl)methyl)- | |
| | 1H-indol-1-yl)acetic acid | |
| 1.104 | 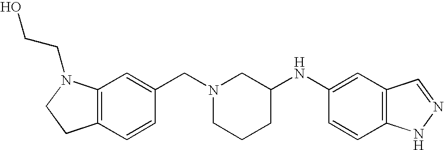 | 1c, 7, 8, 9, 10, 13, 16c |
| | 2-(6-((3-(1H-indazol-5-ylamino)piperidin-1- | |
| | yl)methyl)indolin-1-yl)ethanol | |
| 1.105 | 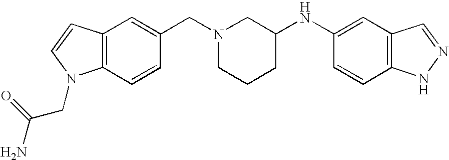 | 1c, 7, 8, 9, 10, 13, 16c |
| | 2-(5-((3-(1H-indazol-5-ylamino)piperidin-1-yl)methyl)- | |
| | 1H-indol-1-yl)acetamide | |
| 1.106 | 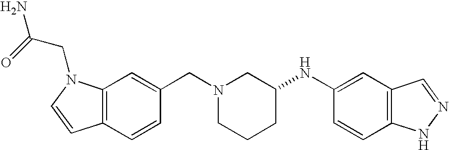 | 1c, 7, 8, 9, 10, 13, 16c |
| | (R)-2-(6-((3-(1H-indazol-5-ylamino)piperidin-1- | |
| | yl)methyl)-1H-indol-1-yl)acetamide | |
| 1.107 | 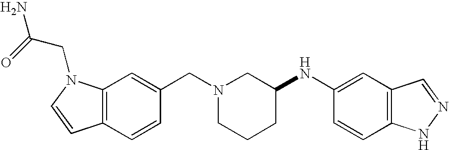 | 1c, 7, 8, 9, 10, 13, 16c |
| | (S)-2-(6-((3-(1H-indazol-5-ylamino)piperidin-1- | |
| | yl)methyl)-1H-indol-1-yl)acetamide | |
| 1.108 | 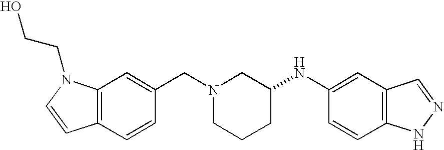 | 1c, 7, 8, 9, 10, 13, 16c |
| | (R)-2-(6-((3-(1H-indazol-5-ylamino)piperidin-1- | |
| | yl)methyl)-1H-indol-1-yl)ethanol | |
| 1.109 | 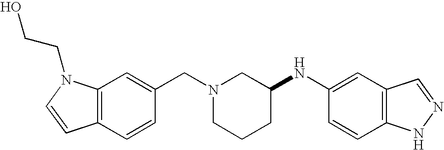 | 1c, 7, 8, 9, 10, 13, 16c |
| | (S)-2-(6-((3-(1H-indazol-5-ylamino)piperidin-1- | |
| | yl)methyl)-1H-indol-1-yl)ethanol | |
| 1.110 | 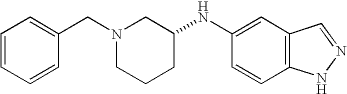 | 1c, 7, 8, 9, 10 |
| | (R)-N-(1-benzylpiperidin-3-yl)-1H-indazol-5-amine | |
| 1.111 | 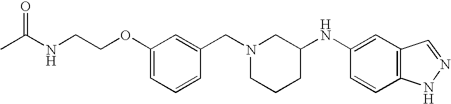 | 1c, 7, 8, 9, 10, 12a, 15c, 15d |
| | N-(2-(3-((3-(1H-indazol-5-ylamino)piperidin-1- | |
| | yl)methyl)phenoxy)ethyl)acetamide | |
| 1.112 | 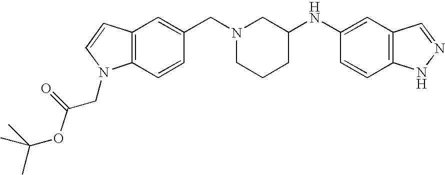 | 1c, 7, 8, 9, 10, 13, 16c |
| | tert-butyl 2-(5-((3-(1H-indazol-5-ylamino)piperidin-1- | |
| | yl)methyl)-1H-indol-1-yl)acetate | |
| 1.113 | 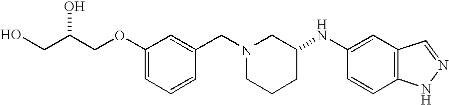 | 1c, 7, 8, 9, 10, 12a, 15c, 16d |
| | (S)-3-(3-(((R)-3-(1H-indazol-5-ylamino)piperidin-1- | |
| | yl)methyl)phenoxy)propane-1,2-diol | |
| 1.114 | 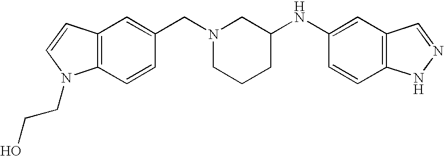 | 1c, 7, 8, 9, 10, 13, 16c |
| | 2-(5-((3-(1H-indazol-5-ylamino)piperidin-1-yl)methyl)- | |
| | 1H-indol-1-yl)ethanol | |
| 1.115 | 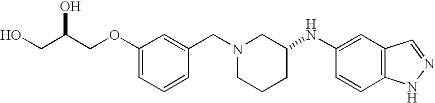 | 1c, 7, 8, 9, 10, 12a, 15c, 15d |
| | (R)-3-(3-(((R)-3-(1H-indazol-5-ylamino)piperidin-1- | |
| | yl)methyl)phenoxy)propane-1,2-diol | |
| 1.116 | 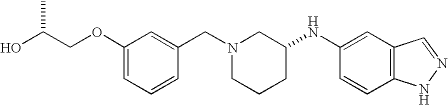 | 1c, 7, 8, 9, 10, 12a, 15c, 15d |
| | (R)-1-(3-(((R)-3-(1H-indazol-5-ylamino)piperidin-1- | |
| | yl)methyl)phenoxy)propan-2-ol | |
| 1.117 | 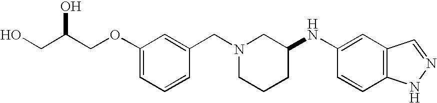 | 1c, 7, 8, 9, 10, 12a, 15c, 15d |
| | (R)-3-(3-(((S)-3-(1H-indazol-5-ylamino)piperidin-1- | |
| | yl)methyl)phenoxy)propane-1,2-diol | |
| 1.118 | 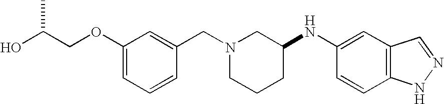 | 1c, 7, 8, 9, 10, 12a, 15c, 15d |
| | (R)-1-(3-(((S)-3-(1H-indazol-5-ylamino)piperidin-1- | |
| | yl)methyl)phenoxy)propan-2-ol | |
| 1.119 | 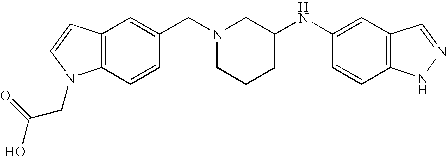 | 1c, 7, 8, 9, 10, 13, 16c |
| | 2-(5-((3-(1H-indazol-5-ylamino)piperidin-l-yl)methyl)- | |
| | 1H-indol-1-yl)acetic acid | |
| 1.120 | 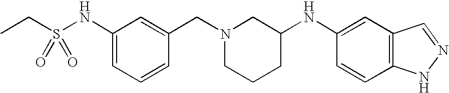 | 1c, 7, 8, 9, 10, 12a, 15c, 15d |
| | N-(3-((3-(1H-indazol-5-yamino)piperidin-1- | |
| | yl)methyl)phenyl)ethanesulfonamide | |
| 1.121 | 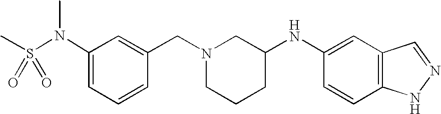 | 1c, 7, 8, 9, 10, 12a, 15c, 15d |
| | N-(3-((3-(1H-indazol-5-ylamino)piperidin-1- | |
| | yl)methyl)phenyl)-N-methylmethanesulfonamide | |
| 1.122 | 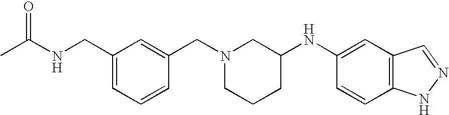 | 1c, 7, 8, 9, 10, 13, 16c |
| | N-(3-((3-(1H-indazol-5-ylamino)piperidin-1- | |
| | yl)methyl)benzyl)acetamide | |
| 1.123 | 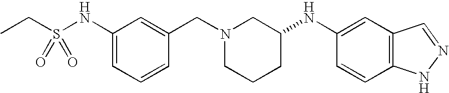 | 1c, 7, 8, 9, 10, 12a, 15c, 15d |
| | (R)-N-(3-((3-(1H-indazol-5-ylamino)piperidin-1- | |
| | yl)methyl)phenyl)ethanesulfonamide | |
| 1.124 | 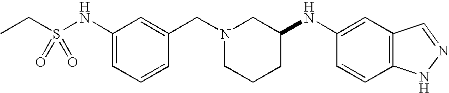 | 1c, 7, 8, 9, 10, 12a, 15c, 15d |
| | (S)-N-(3-((3-(1H-indazol-5-ylamino)piperidin-1- | |
| | yl)methyl)phenyl)ethanesulfonamide | |
| 1.125 | 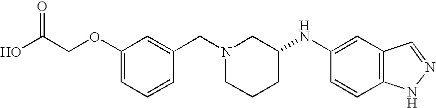 | 1c, 7, 8, 9, 10, 12a, 15c, 15d |
| | (R)-2-(3-((3-(1H-indazol-5-ylamino)piperidin-1- | |
| | yl)methyl)phenoxy)acetic acid | |
| 1.126 | 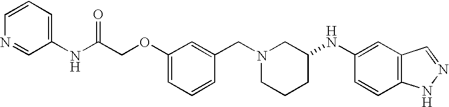 | 1c, 7, 8, 9, 10, 12a, 15c, 15d |
| | (R)-2-(3-((3-(1H-indazol-5-ylamino)piperidin-1- | |
| | yl)methyl)phenoxy)-N-(pyridin-3-yl)acetamide | |
| 1.127 | 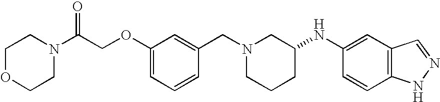 | 1c, 7, 8, 9, 10, 12a, 15c, 15d |
| | (R)-2-(3-((3-(1H-indazol-5-ylamino)piperidin-1- | |
| | yl)methyl)phenoxy)-1-morpholinoethanone | |
| 1.128 | 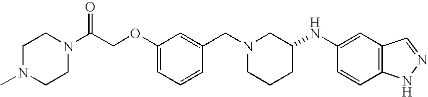 | 1c, 7, 8, 9, 10, 12a, 15c, 15d |
| | (R)-2-(3-((3-(1H-indazol-5-ylamino)piperidin-1- | |
| | yl)methyl)phenoxy)-1-(4-methylpiperazin-1- | |
| | yl)ethanone | |
| 1.129 | 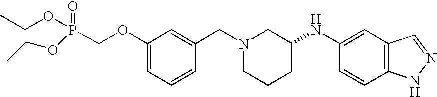 | 1c, 7, 8, 9, 10, 12a, 15c, 15d |
| | (R)-diethyl (3-((3-(1H-indazol-5-ylamino)piperidin-1- | |
| | yl)methyl)phenoxy)methylphosphonate | |
| 1.130 | 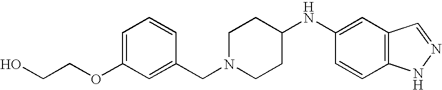 | 1c, 7, 8, 9, 10, 12a, 15c, 15d |
| | 2-(3-((4-(1H-indazol-5-ylamino)piperidin-1- | |
| | yl)methyl)phenoxy)ethanol | |
| 1.131 | 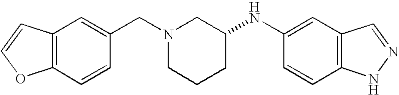 | 1c, 7, 8, 9, 10 |
| | (R)-N-(1-(benzofuran-5-ylmethyl)piperidin-3-yl)-1H- | |
| | indazol-5-amine | |
| 1.132 | 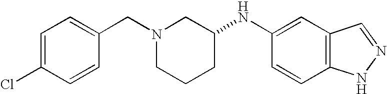 | 1c, 7, 8, 9, 10 |
| | (R)-N-(1-(4-chlorobenzyl)piperidin-3-yl)-1H-indazol-5- | |
| | amine | |
| 1.133 | 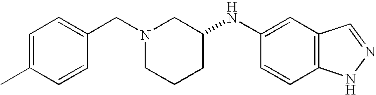 | 1c, 7, 8, 9, 10 |
| | (R)-N-(1-(4-methylbenzyl)piperidin-3-yl)-1H-indazol-5- | |
| | amine | |
| 1.134 | 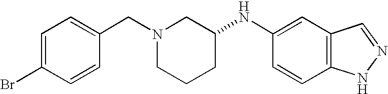 | 1c, 7, 8, 9, 10 |
| | (R)-N-(1-(4-bromobenzyl)piperidin-3-yl)-1H-indazol-5- | |
| | amine | |
| 1.136 | 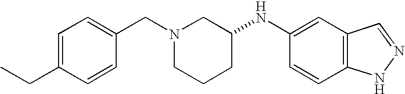 | 1c, 7, 8, 9, 10 |
| | (R)-N-(1-(4-ethylbenzyl)piperidin-3-yl)-1H-indazol-5- | |
| | amine | |
| 1.137 | 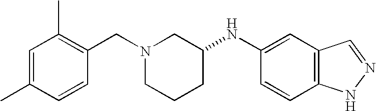 | 1c, 7, 8, 9, 10 |
| | (R)-N-(1-(2,4-dimethylbenzyl)piperidin-3-yl)-1H- | |
| | indazol-5-amine | |
| 1.138 | 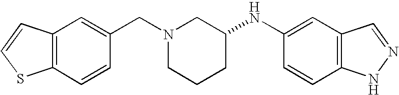 | 1c, 7, 8, 9, 10 |
| | (R)-N-(1-(benzo[b]thiophen-5-ylmethyl)piperidin-3-yl)- | |
| | 1H-indazol-5-amine | |
| 1.139 | 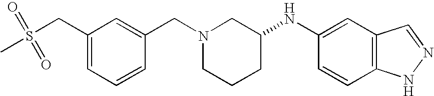 | 1c, 7, 8, 9, 10, 12, 15c |
| | (R)-N-(1-(3-(methylsulfonylmethyl)benzyl)piperidin-3- | |
| | yl)-1H-indazol-5-amine | |
| 1.140 | 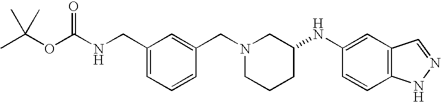 | 1c, 7, 8, 9, 10, 13, 16c |
| | (R)-tert-butyl 3-((3-(1H-indazol-5-ylamino)piperidin-1- | |
| | yl)methyl)benzylcarbamate | |
| 1.141 | 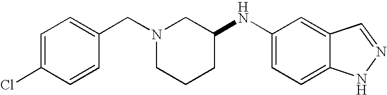 | 1c, 7, 8, 9, 10 |
| | (S)-N-(1-(4-chlorobenzyl)piperidin-3-yl)-1H-indazol-5- | |
| | amine | |
| 1.142 | 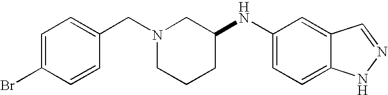 | 1c, 7, 8, 9, 10 |
| | (S)-N-(1-(4-bromobenzyl)piperidin-3-yl)-1H-indazol-5- | |
| | amine | |
| 1.143 | 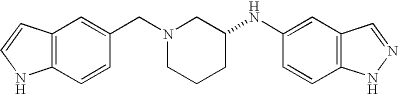 | 1c, 7, 8, 9, 10, 13, 16c |
| | (R)-N-(1-((1-indol-5-yl)methyl)piperidin-3-yl)-1H- | |
| | indazol-5-amine | |
| 1.144 | 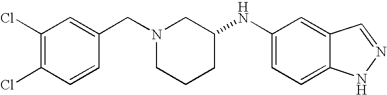 | 1c, 7, 8, 9, 10 |
| | (R)-N-(1-(3,4-dichlorobenzyl)piperidin-3-yl)-1H- | |
| | indazol-5-amine | |
| 1.145 | 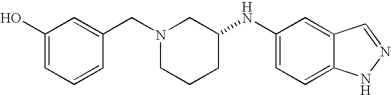 | 1c, 7, 8, 9, 10 |
| | (R)-3-((3-(1H-indazol-5-ylamino)piperidin-1- | |
| | yl)methyl)phenol | |
| 1.146 | 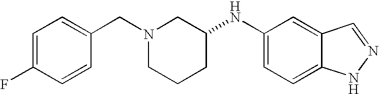 | 1c, 7, 8, 9, 10 |
| | (R)-N-(1-(4-fluorobenzyl)piperidin-3-yl)-1H-indazol-5- | |
| | amine | |
| 1.147 | 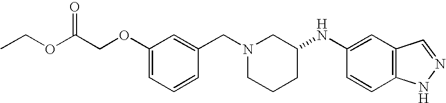 | 1c, 7, 8, 9, 10, 12a, 15c, 15d |
| | (R)-ethyl 2-(3-((3-(1H-indazol-5-ylamino)piperidin-1- | |
| | yl)methyl)phenoxy)acetate | |
| 1.148 | 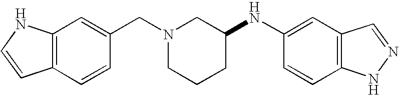 | 1c, 7, 8, 9, 10 |
| | (S)-N-(1-((1H-indol-6-yl)methyl)piperidin-3-yl)-1H- | |
| | indazol-5-amine | |
| 1.149 | 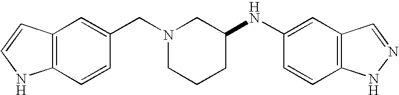 | 1c, 7, 8, 9, 10 |
| | (S)-N-(1-((1H-indol-5-yl)methyl)piperidin-3-yl)-1H- | |
| | indazol-5-amine | |
| 1.150 | 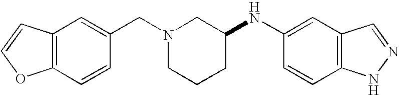 | 1c, 7, 8, 9, 10 |
| | (S)-N-(1-(benzofuran-5-ylmethyl)piperidin-3-yl)-1H- | |
| | indazol-5-amine | |
| 1.151 | 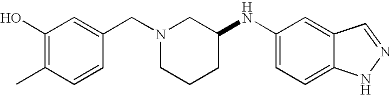 | 1c, 7, 8, 9, 10 |
| | (S)-5-((3-(1H-indazol-5-ylamino)piperidin-1-yl)methyl)- | |
| | 2-methylphenol | |
| 1.152 | 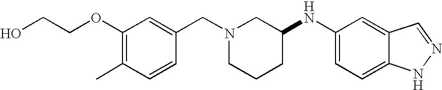 | 1c, 7, 8, 9, 10, 12a, 15c, 15d |
| | (S)-2-(5-((3-(1H-indazol-5-ylamino)piperidin-1- | |
| | yl)methyl)-2-methylphenoxy)ethanol | |
| 1.153 | 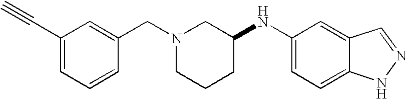 | 1c, 7, 8, 9, 10, 11, 14c |
| | (S)-N-(1-(3-ethynylbenzyl)piperidin-3-yl)-1H-indazol-5- | |
| | amine | |
| 1.154 | 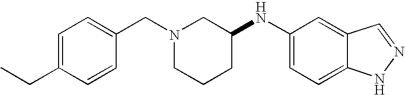 | 1c, 7, 8, 9, 10 |
| | (S)-N-(1-(4-ethylbenzyl)piperidin-3-yl)-1H-indazol-5- | |
| | amine | |
| 1.155 | 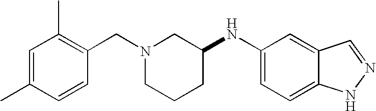 | 1c, 7, 8, 9, 10 |
| | (S)-N-(1-(2,4-dimethylbenzyl)piperidin-3-yl)-1H- | |
| | indazol-5-amine | |
| 1.156 | 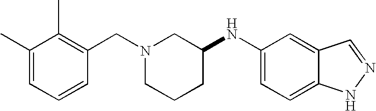 | 1c, 7, 8, 9, 10 |
| | (S)-N-(1-(2,3-dimethylbenzyl)piperidin-3-yl)-1H- | |
| | indazol-5-amine | |
| 1.157 | 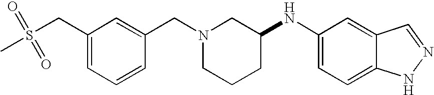 | 1c, 7, 8, 9, 10, 12, 15c |
| | (S)-N-(1-(3-(methylsulfonylmethyl)benzyl)piperidin-3- | |
| | yl)-1H-indazol-5-amine | |
| 1.158 | 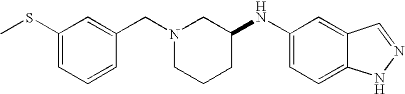 | 1c, 7, 8, 9, 10, 12b, 15c, 15e |
| | (S)-N-(1-(3-(methylthio)benzyl)piperidin-3-yl)-1H- | |
| | indazol-5-amine | |
| 1.159 | 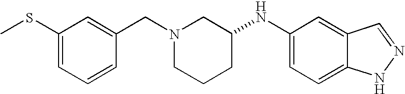 | 1c, 7, 8, 9, 10, 12b, 15c, 15e |
| | (R)-N-(1-(3-(methylthio)benzyl)piperidin-3-yl)-1H- | |
| | indazol-5-amine | |
| 1.160 | 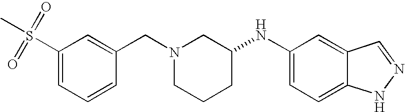 | 1c, 7, 8, 9, 10, 12, 15c |
| | (R)-N-(1-(3-(methylsulfonyl)benzyl)piperidin-3-yl)-1H- | |
| | indazol-5-amine | |
| 1.161 | 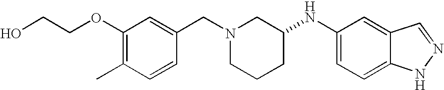 | 1c, 7, 8, 9, 10, 12a, 15c, 15d |
| | (R)-2-(5-((3-(1H-indazol-5-ylamino)piperidin-1- | |
| | yl)methyl)-2-methylphenoxy)ethanol | |
| 1.162 | 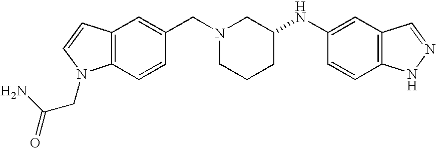 | 1c, 7, 8, 9, 10, 13, 16c |
| | (R)-2-(5-((3-(1H-indazol-5-ylamino)piperidin-1- | |
| | yl)methyl)-1H-indol-1-yl)acetamide | |
| 1.163 | 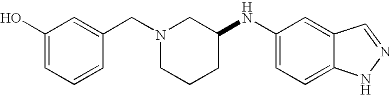 | 1c, 7, 8, 9, 10 |
| | (S)-3-((3-(1H-indazol-5-ylamino)piperidin-1- | |
| | yl)methyl)phenol | |
| 1.164 | 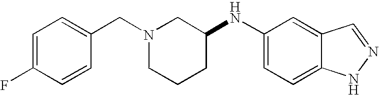 | 1c, 7, 8, 9, 10 |
| | (S)-N-(1-(4-fluorobenzyl)piperidin-3-yl)-1H-indazol-5- | |
| | amine | |
| 1.165 | 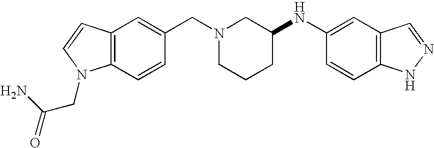 | 1c, 7, 8, 9, 10, 13, 16c |
| | (S)-2-(5-((3-(1H-indazol-5-ylamino)piperidin-1- | |
| | yl)methyl)-1H-indol-1-yl)acetamide | |
| 1.166 | 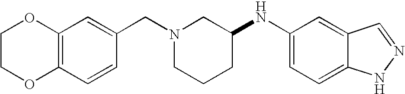 | 1c, 7, 8, 9, 10 |
| | (S)-N-(1-((2,3-dihydrobenzo[1,4]dioxin-6- | |
| | yl)methyl)piperidin-3-yl)-1H-indazol-5-amine | |
| 1.167 | 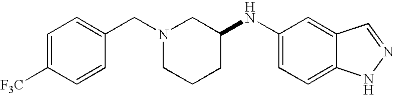 | 1c, 7, 8, 9, 10 |
| | (S)-N-(1-(4-(trifluoromethyl)benzyl)piperidin-3-yl)-1H- | |
| | indazol-5-amine | |
| 1.168 | 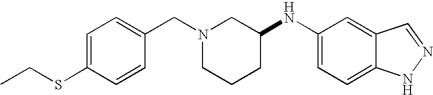 | 1c, 7, 8, 9, 10, 12b, 15c, 15e |
| | (S)-N-(1-(4-(ethylthio)benzyl)piperidin-3-yl)-1H- | |
| | indazol-5-amine | |
| 1.169 | 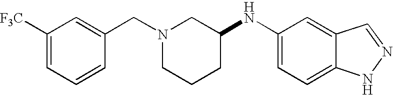 | 1c, 7, 8, 9, 10 |
| | (S)-N-(1-(3-(trifluoromethyl)benzyl)piperidin-3-yl)-1H- | |
| | indazol-5-amine | |
| 1.170 | 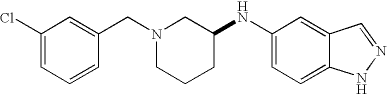 | 1c, 7, 8, 9, 10 |
| | (S)-N-(1-(3-chlorobenzyl)piperidin-3-yl)-1H-indazol-5- | |
| | amine | |
| 1.171 | 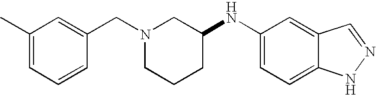 | 1.171 |
| | (S)-N-(1-(3-methylbenzyl)piperidin-3-yl)-1H-indazol-5- | |
| | amine | |
| 1.172 | 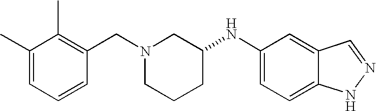 | 1.172 |
| | (R)-N-(1-(2,3-dimethylbenzyl)piperidin-3-yl)-1H- | |
| | indazol-5-amine | |
| 1.173 | 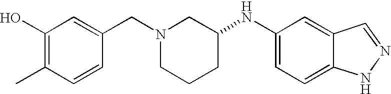 | 1.173 |
| | (R)-5-((3-(1H-indazol-5-ylamino)piperidin-1-yl)methyl)- | |
| | 2-methylphenol | |
| 1.174 | 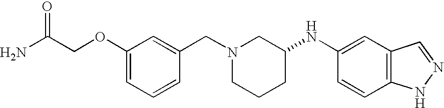 | 1.174 |
| | (R)-2-(3-((3-(1H-indazol-5-ylamino)piperidin-1- | |
| | yl)methyl)phenoxy)acetamide | |
| 1.175 | 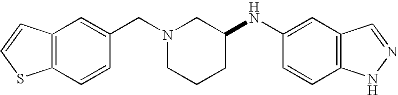 | 1.175 |
| | (S)-N-(1-(benzo[b]thiophen-5-ylmethyl)piperidin-3-yl)- | |
| | 1H-indazol-5-amine | |
| 1.176 | 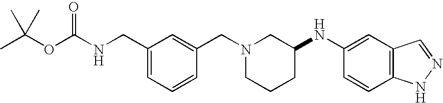 | 1.176 |
| | (S)-tert-butyl 3-((3-(1H-indazol-5-ylamino)piperidin-1- | |
| | yl)methyl)benzylcarbamate | |
| 1.177 | 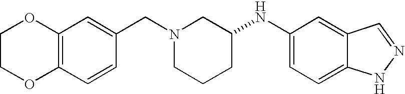 | 1.177 |
| | (R)-N-(1-((2,3-dihydrobenzo[b][1,4]dioxin-6- | |
| | yl)methyl)piperidin-3-yl)-1H-indazol-5-amine | |
| 1.178 | 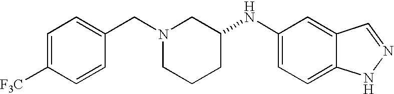 | 1.178 |
| | (R)-N-(1-(4-(trifluoromethyl)benzyl)piperidin-3-yl)-1H- | |
| | indazol-5-amine | |
| 1.179 | 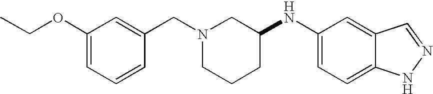 | 1.179 |
| | (S)-N-(1-(3-ethoxybenzyl)piperidin-3-yl)-1H-indazol-5- | |
| | amine | |
| 1.180 | 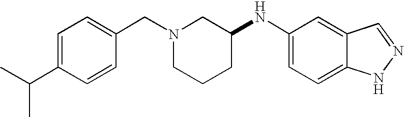 | 1.180 |
| | (S)-N-(1-(4-isopropylbenzyl)piperidin-3-yl)-1H-indazol- | |
| | 5-amine | |
| 1.181 | 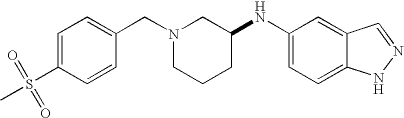 | 1.181 |
| | (S)-N-(1-(4-(methylsulfonyl)benzyl)piperidin-3-yl)-1H- | |
| | indazol-5-amine | |
| 1.182 | 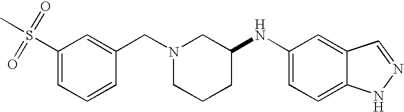 | 1.182 |
| | (S)-N-(1-(3-(methylsulfonyl)benzyl)piperidin-3-yl)-1H- | |
| | indazol-5-amine | |
| 1.183 | 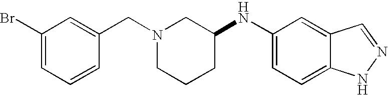 | 1.183 |
| | (S)-N-(1-(3-bromobenzyl)piperidin-3-yl)-1H-indazol-5- | |
| | amine | |
| 1.184 | 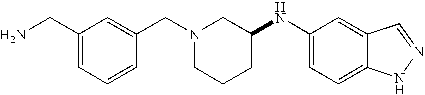 | 1.184 |
| | (S)-N-(1-(3-(aminomethyl)benzyl)piperidin-3-yl)-1H- | |
| | indazol-5-amine | |
| 1.185 | 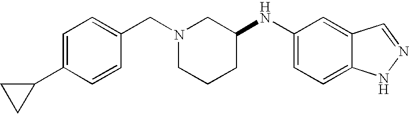 | 1.185 |
| | (S)-N-(1-(4-cyclopropylbenzyl)piperidin-3-yl)-1H- | |
| | indazol-5-amine | |
| 1.186 | 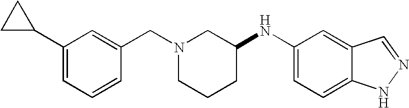 | 1.186 |
| | (S)-N-(1-(3-cyclopropylbenzyl)piperidin-3-yl)-1H- | |
| | indazol-5-amine | |
| 1.187 | 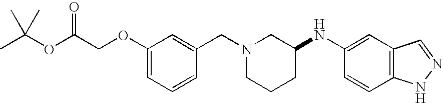 | 1.187 |
| | (S)-tert-butyl 2-(3-((3-(1H-indazol-5-ylamino)piperidin- | |
| | 1-yl)methyl)phenoxy)acetate | |
| 1.188 | 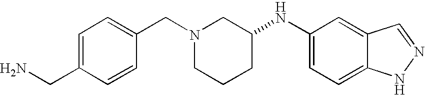 | 1.188 |
| | (R)-N-(1-(4-(aminomethyl)benzyl)piperidin-3-yl)-1H- | |
| | indazol-5-amine | |
| 1.189 | 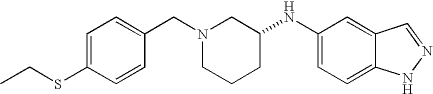 | 1.189 |
| | (R)-N-(1-(4-(ethylthio)benzyl)piperidin-3-yl)-1H- | |
| | indazol-5-amine | |
| 1.190 | 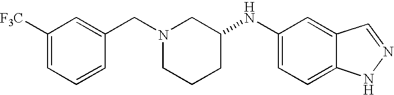 | 1.190 |
| | (R)-N-(1-(3-(trifluoromethyl)benzyl)piperidin-3-yl)-1H- | |
| | indazol-5-amine | |
| 1.191 | 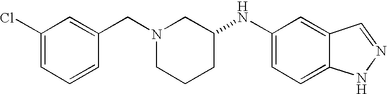 | 1c, 7, 8, 9, 10 |
| | (R)-N-(1-(3-chlorobenzyl)piperidin-3-yl)-1H-indazol-5- | |
| | amine | |
| 1.192 | 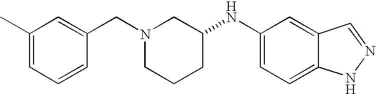 | 1c, 7, 8, 9, 10 |
| | (R)-N-(1-(3-methylbenzyl)piperidin-3-yl)-1H-indazol-5- | |
| | amine | |
| 1.193 | 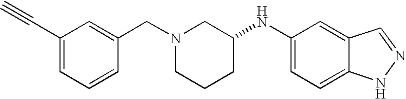 | 1c, 7, 8, 9, 10, 11, 14c |
| | (R)-N-(1-(3-ethynylbenzyl)piperidin-3-yl)-1H-indazol-5- | |
| | amine | |
| 1.194 | 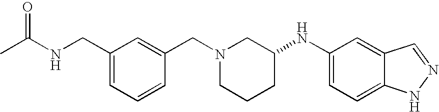 | 1c, 7, 8, 9, 10, 13, 16c |
| | (R)-N-(1H-indazol-5-ylamino)piperidin-1- | |
| | yl)methyl)benzyl)acetamide | |
| 1.195 | 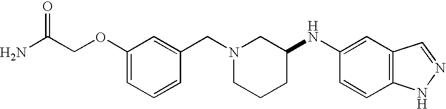 | 1c, 7, 8, 9, 10, 12a, 15c, 15d |
| | (S)-2-(3-((3-(1H-indazol-5-ylamino)piperidin-1- | |
| | yl)methyl)phenyl-1H-indazol-5-ylamino)piperidin-1- | |
| | yl)methyl)phenoxy)acetamide | |
| 1.196 | 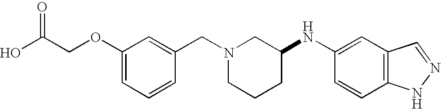 | 1c, 7, 8, 9, 10, 12a, 15c, 15d |
| | (S)-2-(3-((3-(1H-indazol-5-ylamino)piperidin-1- | |
| | yl)methyl)phenoxy)acetic acid | |
| 1.197 | 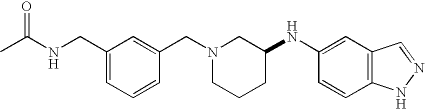 | 1c, 7, 8, 9, 10, 13, 16c |
| | (S)-N-(1H-indazol-5-ylamino)piperidin-1- | |
| | yl)methyl)benzyl)acetamide | |
| 1.198 | 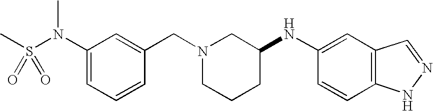 | 1c, 7, 8, 9, 10, 12a, 15c, 15d |
| | (S)-N-(3-((3-(1H-indazol-5-ylamino)piperidin-1- | |
| | yl)methyl)phenyl)-N-methylmethanesulfonamide | |
| 1.199 | 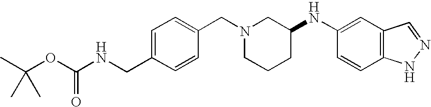 | 1c, 7, 8, 9, 10, 13, 16c |
| | (S)-tert-butyl 4-((3-(1H-indazol-5-ylamino)piperidin-1- | |
| | yl)methyl)benzylcarbamate | |
| 1.200 | 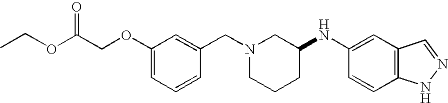 | 1c, 7, 8, 9, 10, 12a, 15c, 15d |
| | (S)-ethyl 2-(3-((3-(1H-indazol-5-ylamino)piperidin-1- | |
| | yl)methyl)phenoxy)acetate | |
| 1.201 | 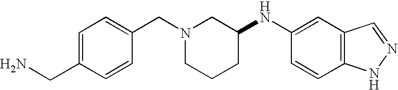 | 1c, 7, 8, 9, 10, 13, 16c |
| | (S)-N-(1-(4-(aminomethyl)benzyl)piperidin-3-yl)-1H- | |
| | indazol-5-amine | |
| 1.202 | 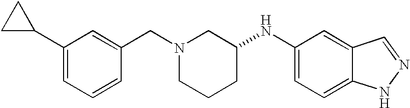 | 1c, 7, 8, 9, 10, 11, 14c |
| | (R)-N-(1-(3-cyclopropylbenzyl)piperidin-3-yl)-1H- | |
| | indazol-5-amine | |
| 1.203 | 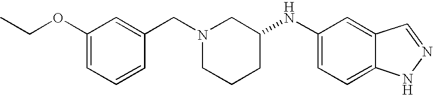 | 1c, 7, 8, 9, 10 |
| | (R)-N-(1-(3-ethoxybenzyl)piperidin-3-yl)-1H-indazol-5- | |
| | amine | |
| 1.204 | 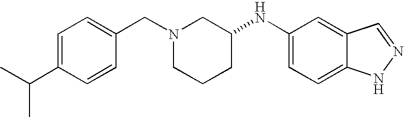 | 1c, 7, 8, 9, 10 |
| | (R)-N-(1-(4-Isopropylbenzyl)piperidin-3-yl)-1H-indazol- | |
| | 5-amine | |
| 1.205 | 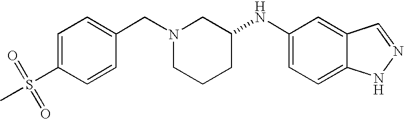 | 1c, 7, 8, 9, 10, 12, 15c |
| | (R)-N-(1-(4-(methylsulfonyl)benzyl)piperidin-3-yl)-1H- | |
| | indazol-5-amine | |
| 1.206 | 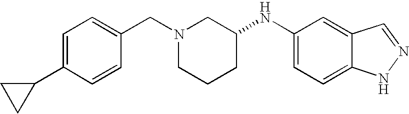 | 1c, 7, 8, 9, 10, 11, 14c |
| | (R)-N-(1-(4-cyclopropylbenzyl)piperidin-3-yl)-1H- | |
| | indazol-5-amine | |
| 1.207 | 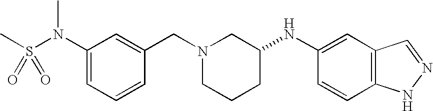 | 1c, 7, 8, 9, 120, 12a, 15c, 15d |
| | (R)-N-(3-((3-(1H-indazol-5-ylamino)piperidin-1- | |
| | yl)methyl)phenyl)-N-methylmethanesulfonamide | |
| 1.208 | 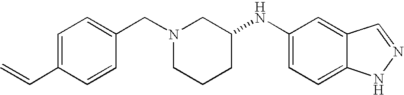 | 1c, 7, 8, 9, 10, 11, 14c |
| | (R)-N-(1-(4-vinylbenzyl)piperidin-3-yl)-1H-indazol-5- | |
| | amine | |
| 1.209 | 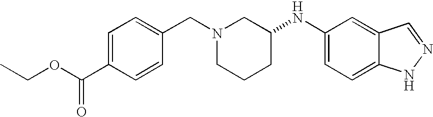 | 1c, 7, 8, 9, 10 |
| | (R)-ethyl 4-((3-(1H-indazol-5-ylamino)piperidin-1- | |
| | yl)methyl)benzoate | |
| 1.210 | 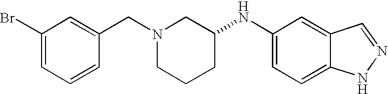 | 1c, 7, 8, 9, 10 |
| | (R)-N-(1-(3-bromobenzyl)piperidin-3-yl)-1H-indazol-5- | |
| | amine | |
| 1.211 | 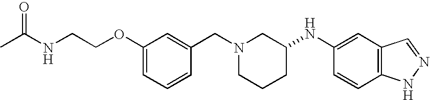 | 1c, 7, 8, 9, 10, 12a, 15c, 15d |
| | (R)-N-(2-(3-((3-(1H-indazol-5-ylamino)piperidin-1- | |
| | yl)methyl)phenoxy)ethyl)acetamide | |
| 1.212 | 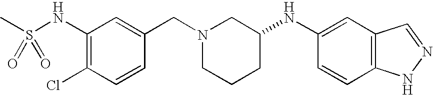 | 1c, 7, 8, 9, 10, 12a, 15c, 15d |
| | (R)-N-(5-((3-(1H-indazol-5-ylamino)piperidin-1- | |
| | yl)methyl)-2-chlorophenyl)methanesulfonamide | |
| 1.213 | 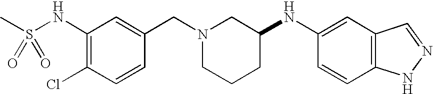 | 1c, 7, 8, 9, 10, 12a, 15c, 15d |
| | (S)-N-(5-((3-(1H-indazol-5-ylamino)piperidin-1- | |
| | yl)methyl)-2-chlorophenyl)methanesulfonamide | |
| 1.214 | 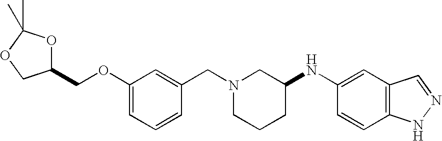 | 1c, 7, 8, 9, 10, 12a, 15c, 15d |
| | N-((S)-1-(3-(((S)-2,2-dimethyl-1,3-dioxolan-4- | |
| | yl)methoxy)benzyl)piperidin-3-yl)-1H-indazol-5-amine | |
| 1.215 | 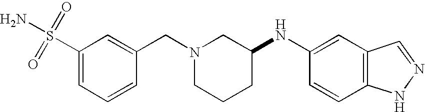 | 1c, 7, 8, 9, 10, 12, 15c |
| | (S)-3-((3-(1H-indazol-5-ylamino)piperidin-1- | |
| | yl)methyl)benzenesulfonamide | |
| 1.216 | 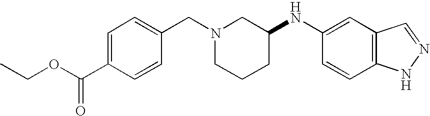 | 1c, 7, 8, 9, 10 |
| | (S)-ethyl 4-((3-(1H-indazol-5-ylamino)piperidin-1- | |
| | yl)methyl)benzoate | |
| 1.217 | 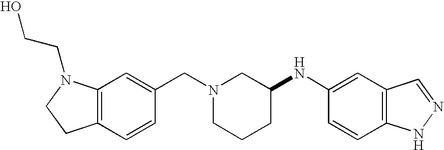 | 1c, 7, 8, 9, 10, 13, 16c |
| | (S)-2-(6-((3-(1H-indazol-5-ylamino)piperidin-1- | |
| | yl)methyl)indolin-1-yl)ethanol | |
| 1.218 | 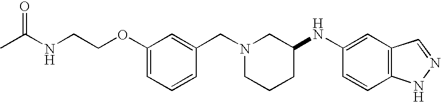 | 1c, 7, 8, 9, 10, 12a, 15c, 15d |
| | (S)-N-(2-(3-((3-(1H-indazol-5-ylamino)piperidin-1- | |
| | yl)methyl)phenoxy)ethyl)acetamide | |
| 1.219 | 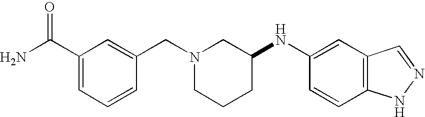 | 1c, 7, 8, 9, 10, 12, 15c |
| | (S)-3-((3-(1H-indazol-5-ylamino)piperidin-1- | |
| | yl)methyl)benzamide | |
| 1.221 | 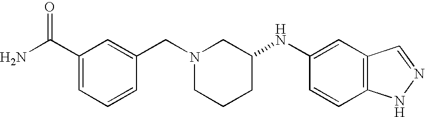 | 1c, 7, 8, 9, 10, 12, 15c |
| | (R)-3-((3-(1H-indazol-5-ylamino)piperidin-1- | |
| | yl)methyl)benzamide | |
| 1.222 | 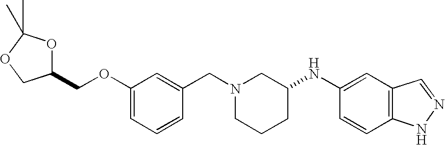 | 1c, 7, 8, 9, 10, 12a, 15c, 15d |
| | N-((R)-1-(3-(((S)-2,2-dimethyl-1,3-dioxolan-4- | |
| | yl)methoxy)benzyl)piperidin-3-yl)-1H-indazol-5-amine | |
| 1.223 | 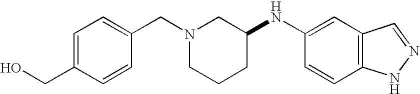 | 1c, 7, 8, 9, 10, 13, 16c |
| | (S)-(4-((3-(1H-indazol-5-ylamino)piperidin-1- | |
| | yl)methyl)phenyl)methanol | |
| 1.224 | 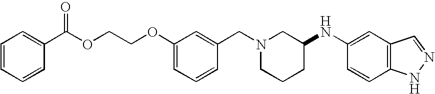 | 1c, 7, 8, 9, 10, 12a, 15c, 15d |
| | (S)-2-(3-((3-(1H-indazol-5-ylamino)piperidin-1- | |
| | yl)methyl)phenoxy)ethyl benzoate | |
| 1.225 | 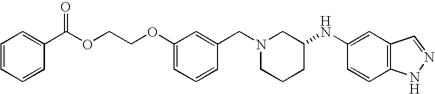 | 1c, 7, 8, 9, 10, 12a, 15c, 15d |
| | (R)-2-(3-((3-(1H-indazol-5-ylamino)piperidin-1- | |
| | yl)methyl)phenoxy)ethyl benzoate | |
| 1.226 | 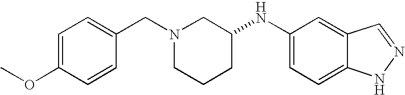 | 1c, 7, 8, 9, 10 |
| | (R)-N-(l-(4-methoxybenzyl)piperidin-3-yl)-1H-indazol- | |
| | 5-amine | |
| 1.227 | 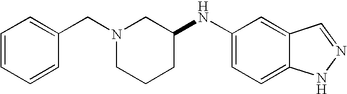 | 1c, 7, 8, 9, 10 |
| | (S)-N-(1-benzylpiperidin-3-yl)-1H-indazol-5-amine | |
| 1.228 | 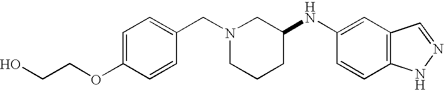 | 1c, 7, 8, 9, 10, 12a, 15c, 15d |
| | (S)-2-(4-((3-(1H-indazol-5-ylamino)piperidin-1- | |
| | yl)methyl)phenoxy)ethanol | |
| 1.229 | 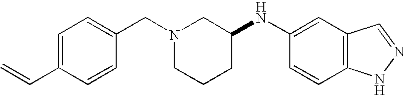 | 1c, 7, 8, 9, 10, 11, 14c |
| | (S)-N-(1-(4-vinylbenzyl)piperidin-3-yl)-1H-indazol-5- | |
| | amine | |
| 1.230 | 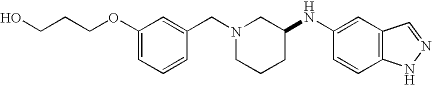 | 1c, 7, 8, 9, 10, 12a, 15c, 15d |
| | (S)-3-(3-((3-(1H-indazol-5-ylamino)piperidin-1- | |
| | yl)methyl)phenoxy)propan-1-ol | |
| 1.231 | 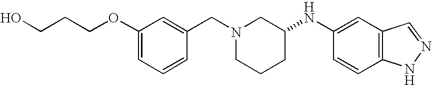 | 1c, 7, 8, 9, 10, 12a, 15c, 15d |
| | (R)-3-(3-((3-(1H-indazol-5-ylamino)piperidin-1- | |
| | yl)methyl)phenoxy)propan-1-ol | |
| 1.232 | 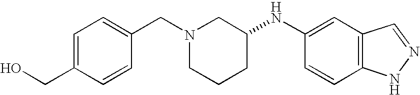 | 1c, 7, 8, 9, 10 |
| | (R)-(4-((3-(1H-indazol-5-ylamino)piperidin-1- | |
| | yl)methyl)phenyl)methanol | |
| 1.233 | 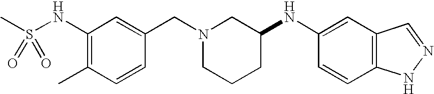 | 1c, 7, 8, 9, 10, 12a, 15c, 15d |
| | (S)-N-(5-((3-(1H-indazol-5-ylamino)piperidin-1- | |
| | yl)methyl)-2-methylphenyl)methanesulfonamide | |
| 1.234 | 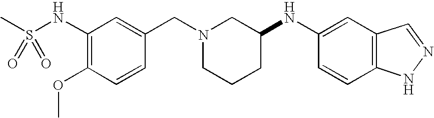 | 1c, 7, 8, 9, 10, 12a, 15c, 15d |
| | (S)-N-(5-((3-(1H-indazol-5-ylamino)piperidin-1- | |
| | yl)methyl)-2-methoxyphenyl)methanesulfonamide | |
| 1.235 | 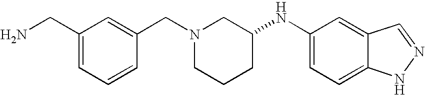 | 1c, 7, 8, 9, 10, 13, 16c |
| | (R)-N-(1-(3-(aminomethyl)benzyl)piperidin-3-yl)-1H- | |
| | indazol-5-amine | |
| 1.236 | 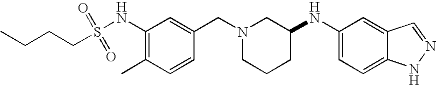 | 1c, 7, 8, 9, 10, 12a, 15c, 15d |
| | (S)-N-(5-((3-(1H-indazol-5-ylamino)piperidin-1- | |
| | yl)methyl)-2-methylphenyl)butane-1-sulfonamide | |
| 1.237 | 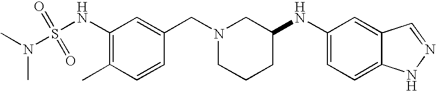 | 1c, 7, 8, 9, 10, 12a, 15c, 15d |
| | (S)-N-(2-((3-(1H-indazol-5-ylamino)piperidin-1- | |
| | yl)methyl)-5-methylphenyl)-N′,N′ | |
| | dimethylaminosulfamide | |
| 1.238 | 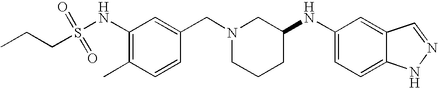 | 1c, 7, 8, 9, 10, 12a, 15c, 15d |
| | (S)-N-(5-((3-(1H-indazol-5-ylamino)piperidin-1- | |
| | yl)methyl)-2-methylphenyl)propane-1-sulfonamide | |
| 1.239 | 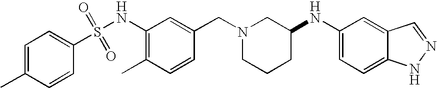 | 1c, 7, 8, 9, 10, 12a, 15c, 15d |
| | (S)-N-(5-((3-(1H-indazol-5-ylamino)piperidin-1- | |
| | yl)methyl)-2-methylphenyl)-4- | |
| | methylbenzenesulfonamide | |
| 1.240 | 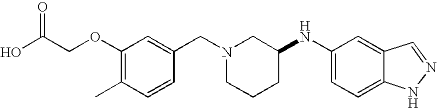 | 1c, 7, 8, 9, 10, 12a, 15c, 15d |
| | (S)-2-(5-((3-(1H-indazol-5-ylamino)piperidin-1- | |
| | yl)methyl)-2-methylphenyl-1H-indazol-5- | |
| | ylamino)piperidin-1-yl)methyl)-2-methylphenoxy)acetic | |
| | acid | |
| 1.241 | 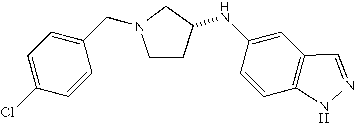 | 1c, 7, 8, 9, 10 |
| | (R)-N-(1-(4-chlorobenzyl)pyrrolidin-3-yl)-1H-indazol-5- | |
| | amine | |
| 1.242 | 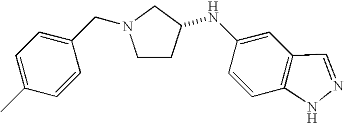 | 1c, 7, 8, 9, 10 |
| | (R)-N-(1-(4-methylbenzyl)pyrrolidin-3-yl)-1H-indazol-5- | |
| | amine | |
| 1.243 | 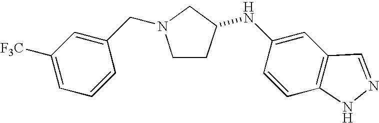 | 1c, 7, 8, 9, 10 |
| | (R)-N-(1-(3-(trifluoromethyl)benzyl)pyrrolidin-3-yl)-1H- | |
| | indazol-6-amine | |
| 1.244 | 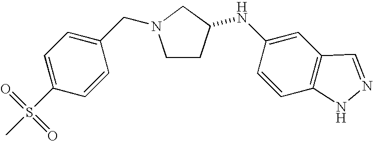 | 1c, 7, 8, 9, 10, 12b, 15c, 15e |
| | (R)-N-(1-(4-(methylsulfonyl)benzyl)pyrrolidin-3-yl)-1H- | |
| | indazol-5-amine | |
| 1.245 | 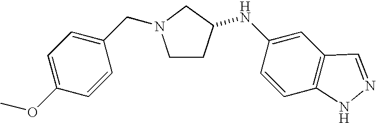 | 1c, 7, 8, 9, 10 |
| | (R)-N-(1-(4-methoxybenzyl)pyrrolidin-3-yl)-1H-indazol- | |
| | 5-amine | |
| 1.246 | 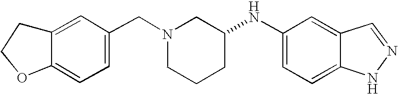 | 1c, 7, 8, 9, 10 |
| | (R)-N-(1-((2,3-dihydrobenzofuran-5- | |
| | yl)methyl)piperidin-3-yl)-1H-indazol-5-amine | |
| 1.247 | 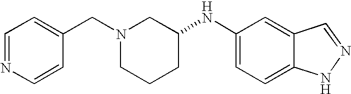 | 1c, 7, 8, 9, 10 |
| | (R)-N-(1-(pyridin-4-ylmethyl)piperidin-3-yl)-1H-indazol- | |
| | 5-amine | |
| 1.248 | 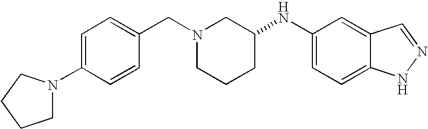 | 1c, 7, 8, 9, 10, 11, 14c |
| | (R)-N-(1-(4-(pyrrolidin-1-yl)benzyl)piperidin-3-yl)-1H- | |
| | indazol-5-amine | |
| 1.249 | 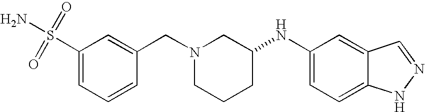 | 1c, 7, 8, 9, 10, 12b, 15c, 15e |
| | (R)-3-((3-(1H-indazol-5-ylamino)piperidin-1- | |
| | yl)methyl)benzenesulfonamide | |
| 1.250 | 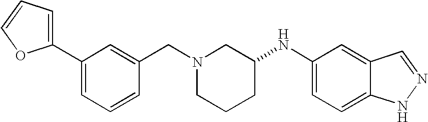 | 1c, 7, 8, 9, 10, 11, 14c |
| | (R)-N-(1-(3-(furan-2-yl)benzyl)piperidin-3-yl)-1H- | |
| | indazol-5-amine | |
| 1.251 | 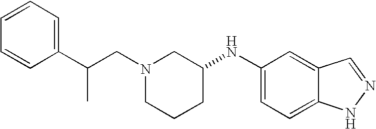 | 1c, 7, 8, 9 |
| | N-((3R)-1-(2-phenylpropyl)piperidin-3-yl)-1H-indazol- | |
| | 5-amine | |
| 1.252 | 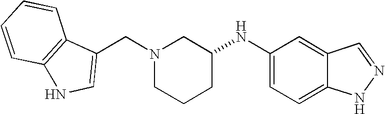 | 1c, 7, 8, 9, 10 |
| | (R)-N-(1-((1H-indol-3-yl)methyl)piperidin-3-yl)-1H- | |
| | indazol-5-amine | |
| 1.253 | 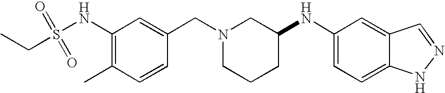 | 1c, 7, 8, 9, 10, 12a, 15c, 15d |
| | (S)-N-(5-((3-(1H-indazol-5-ylamino)piperidin-1- | |
| | yl)methyl)-2-methylphenyl)ethanesulfonamide | |
| 1.254 | 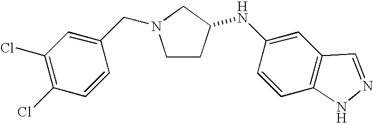 | 1c, 7, 8, 9, 10 |
| | (R)-N-(1-(3,4-dichlorobenzyl)pyrrolidin-3-yl)-1H- | |
| | indazol-5-amine | |
| 1.255 | 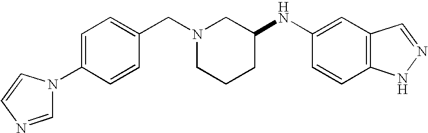 | 1c, 7, 8, 9, 10, 11, 14c |
| | (S)-N-(1-(1H-imidazol-1-yl)benzyl)piperidin-3-yl)-1H- | |
| | indazol-5-amine | |
| 1.256 | 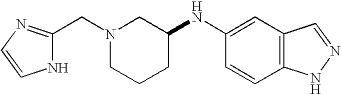 | 1c, 7, 8, 9, 10 |
| | (S)-N-(1-((1H-imidazol-2-yl)methyl)piperidin-3-yl)-1H- | |
| | indazol-5-amine | |
| 1.257 | 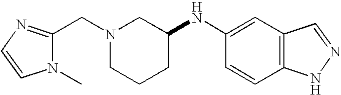 | 1c, 7, 8, 9, 10 |
| | (S)-N-(1-((1-methyl-1H-imidazol-2-yl)methyl)piperidin- | |
| | 3-yl)-1H-indazol-5-amine | |
| 1.258 | 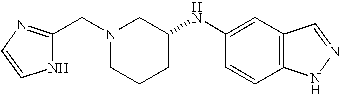 | 1c, 7, 8, 9, 10, 12a, 15c, 15d |
| | (R)-N-(5-((3-(1H-indazol-5-ylamino)piperidin-1- | |
| | yl)methyl)-2-methylphenyl)methanesulfonamide | |
| 1.259 | 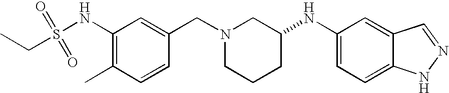 | 1c, 7, 8, 9, 10, 12a, 15c, 15d |
| | (R)-N-(5-((3-(1H-indazol-5-ylamino)piperidin-1- | |
| | yl)methyl)-2-methylphenyl)ethanesulfonamide | |
| 1.260 | 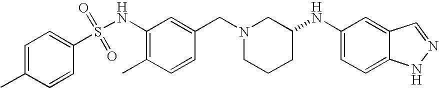 | 1c, 7, 8, 9, 10, 12a, 15c, 15d |
| | (R)-N-(5-((3-(1H-indazol-5-ylamino)piperidin-1- | |
| | yl)methyl)-2-methylphenyl)-4- | |
| | methylbenzenesulfonamide | |
| 1.261 | 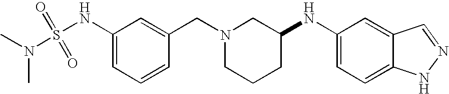 | 1c, 7, 8, 9, 10, 12a, 15c, 15d |
| | (S)-N-(3-((3-(1H-indazol-5-ylamino)piperidin-1- | |
| | yl)methyl)phenyl)-N′,N′dimethylaminosulfamide | |
| 1.262 | 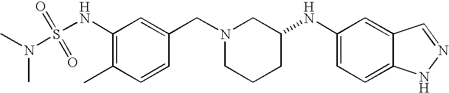 | 1c, 7, 8, 9, 10, 12a, 15c, 15d |
| | (R)-N-(2-((3-(1H-indazol-5-ylamino)piperidin-1- | |
| | yl)methyl)-5-methylphenyl)-N′,N′ | |
| | dimethylaminosulfamide | |
| 1.263 | 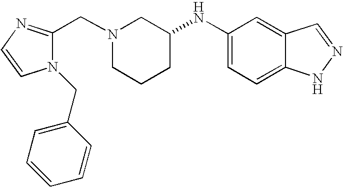 | 1c, 7, 8, 9, 10, 11, 14c |
| | (R)-N-(1-((1-benzyl-1H-imidazol-2-yl)methyl)piperidin- | |
| | 3-yl)-1H-indazol-5-amine | |
| 1.264 | 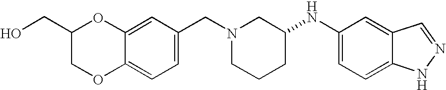 | 1c, 7, 8, 9, 10, 13, 16c |
| | (7-(((R)-3-(1H-indazol-5-ylamino)piperidin-1- | |
| | yl)methyl)-2,3-dihydrobenzo[b][1,4]dioxin-2- | |
| | yl)methanol | |
| 1.265 | 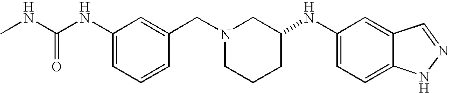 | 1c, 7, 8, 9, 10, 12a, 15c, 15d |
| | (R)-1-(3-((3-(1H-indazol-5-ylamino)piperidin-1- | |
| | yl)methyl)phenyl)-3-methylurea | |
| 1.266 | 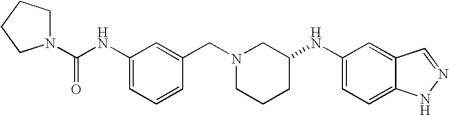 | 1c, 7, 8, 9, 10, 12a, 15c, 15d |
| | (R)-N-(3-((3-(1H-indazol-5-ylamino)piperidin-1- | |
| | yl)methyl)phenyl)pyrrolidine-1-carboxamide | |
| 1.267 | 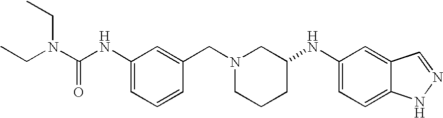 | 1c, 7, 8, 9, 10, 12a. 15c, 15d |
| | (R)-3-(3-((3-(lH-indazol-5-ylamino)piperidin-1- | |
| | yl)methyl)phenyl)-l,1-diethylurea | |
| 1.268 | 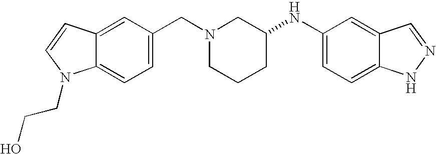 | 1c, 7, 8, 9, 10, 13, 16c |
| | (R)-2-(5-((3-(1H-indazol-5-ylamino)piperidin-1- | |
| | yl)methyl)-1H-indol-1-yl)ethanol | |
| 1.269 | 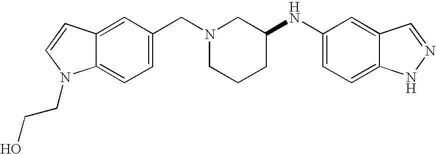 | 1c, 7, 8, 9, 10, 13, 16c |
| | (S)-2-(5-((3-(1H-indazol-5-ylamino)piperidin-1- | |
| | yl)methyl)-1H-indol-1-yl)ethanol | |
| 1.270 | 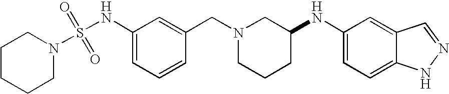 | 1c, 7, 8, 9, 10, 12a, 15c, 15d |
| | (S)-N-(3-((3-(1H-indazol-5-ylamino)piperidin-1- | |
| | yl)methyl)phenyl)piperidine-1-sulfonamide | |
| 1.271 | 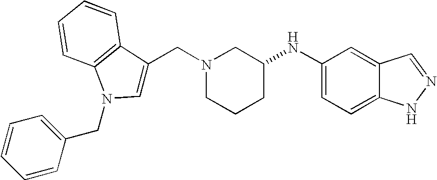 | 1c, 7, 8, 9, 10, 11, 14c |
| | (R)-N-(1-((1-benzyl-1H-indol-3-yl)methyl)piperidin-3- | |
| | yl)-1H-indazol-5-amine | |
| 1.272 | 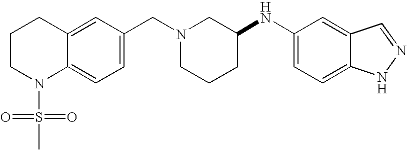 | 1c, 7, 8, 9, 10, 12b, 15c, 15e |
| | (S)-N-(1-((1-(methylsulfonyl)-1,2,3,4- | |
| | tetrahydroquinolin-6-yl)methyl)piperidin-3-yl)-1H- | |
| | indazol-5-amine | |
| 1.273 | 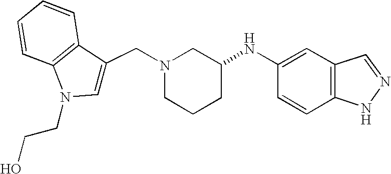 | 1c, 7, 8, 9, 10, 13, 16c |
| | (R)-2-(3-((3-(1H-indazol-5-ylamino)piperidin-1- | |
| | yl)methyl)-1H-indol-1-yl)ethanol | |
| 1.274 | 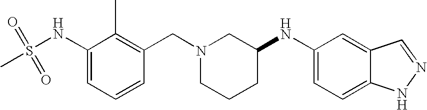 | 1c, 7, 8, 9, 10, 12a, 15c, 15d |
| | (S)-N-(3-((3-(1H-indazol-5-ylamino)piperidin-1- | |
| | yl)methyl)-2-methylphenyl)methanesulfonamide | |
| 1.275 | 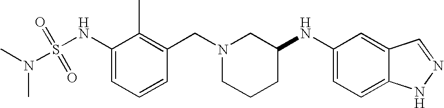 | 1c, 7, 8, 9, 10, 12a, 15c, 15d |
| | (S)-N-(3-((3-(1H-indazol-5-ylamino)piperidin-1- | |
| | yl)methyl)-2-methylphenyl)-N′,N′ | |
| | dimethylaminosulfamide | |
| 1.276 | 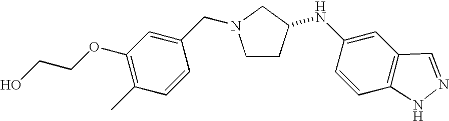 | 1c, 7, 8, 9, 10, 12a, 15c, 15d |
| | (R)-2-(5-((3-(1H-indazol-5-ylamino)pyrrolidin-1- | |
| | yl)methyl)-2-methylphenyl-1H-indazol-5- | |
| | ylamino)pyrrolidin-1-yl)methyl)-2- | |
| | methylphenoxy)ethanol | |
| 1.277 | 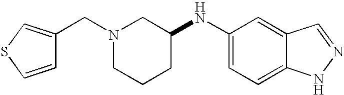 | 1c, 7, 8, 9, 10 |
| | (S)-N-(1-(thiophen-3-ylmethyl)piperidin-3-yl)-1H- | |
| | indazol-5-amine | |
| 1.278 | 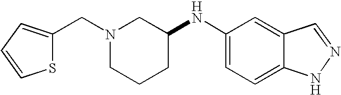 | 1c, 7, 8, 9, 10 |
| | (S)-N-(1-(thiophen-2-ylmethyl)piperidin-3-yl)-1H- | |
| | indazol-5-amine | |
| 1.279 | 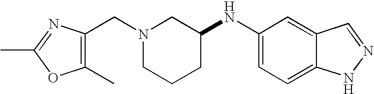 | 1c, 7, 8, 9, 10 |
| | (S)-N-(1-((2,5-dimethyloxazol-4-yl)methyl)piperidin-3- | |
| | yl)-1H-indazol-5-amine | |
| 1.280 | 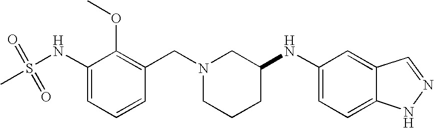 | 1c, 7, 8, 9, 10, 12a, 15c, 15d |
| | (S)-N-(3-((3-(1H-indazol-5-ylamino)piperidin-1- | |
| | yl)methyl)-2-methoxyphenyl)methanesulfonamide | |
| 1.281 | 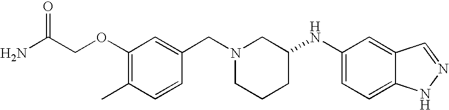 | 1c, 7, 8, 9, 10, 12a, 15c, 15d |
| | (R)-2-(S-((3-(1H-indazol-5-ylamino)piperidin-1- | |
| | yl)methyl)-2-methylphenyl-1H-indazol-5- | |
| | ylamino)piperidin-1-yl)methyl)-2- | |
| | methylphenoxy)acetamide | |
| 1.282 | 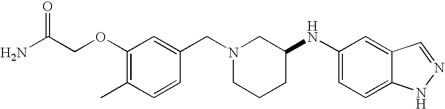 | 1c, 7, 8, 9, 10, 12a, 15c, 15d |
| | (S)-2-(5-((3-(1H-indazol-5-ylamino)piperidin-1- | |
| | yl)methyl)-2-methylphenyl-1H-indazol-5- | |
| | ylamino)piperidin-1-yl)methyl)-2- | |
| | methylphenoxy)acetamide | |
| 2.001 | 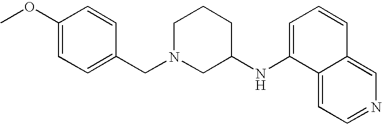 | 2c, 7, 8, 9, 10 |
| | N-(1-(4-methoxybenzyl)piperidin-3-yl)isoquinolin-5- | |
| | amine | |
| 2.002 | 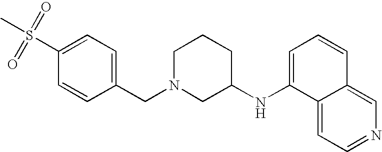 | 2c, 7, 8, 9, 10, 12, 15c |
| | N-(1-(4-(methylsulfonyl)benzyl)piperidin-3- | |
| | yl)isoquinolin-5-amine | |
| 2.003 | 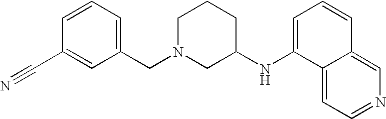 | 2c, 7, 8, 9, 10, 12, 15c |
| | 3-((3-(isoquinolin-5-ylamino)piperidin-1- | |
| | yl)methyl)benzonitrile | |
| 2.004 | 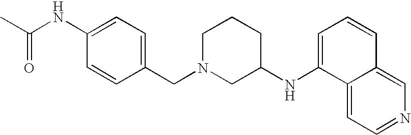 | 2c, 7, 8, 9, 10, 12a, 15c, 15d |
| | N-(4-((3-(isoquinolin-5-ylamino)piperidin-1- | |
| | yl)methyl)phenyl)acetamide | |
| 2.005 | 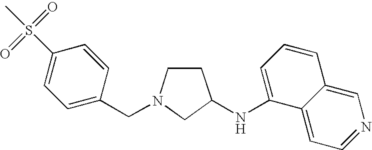 | 2c, 7, 8, 9, 10, 12, 15c |
| | N-(1-(4-(methylsulfonyl)benzyl)pyrrolidin-3- | |
| | yl)isoguinolin-5-amine | |
| 2.006 | 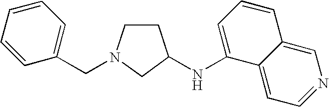 | 2c, 7, 8, 9, 10 |
| | N-(1-benzylpyrrolidin-3-yl)isoquinolin-5-amine | |
| 2.007 | 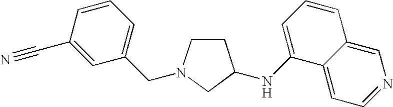 | 2c, 7, 8, 9, 10, 12, 15c |
| | 3-((3-(isoquinolin-5-ylamino)pyrrolidin-1- | |
| | yl)methyl)benzonitrile | |
| 2.008 | 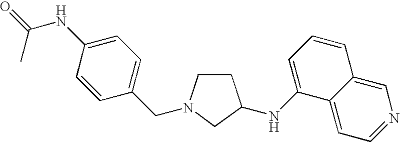 | 2c, 7, 8, 9, 10, 12a, 15c, 15d |
| | N-(4-((3-(isoquinolin-5-ylamino)pyrrolidin-1- | |
| | yl)methyl)phenyl)acetamide | |
| 2.009 | 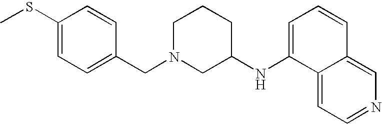 | 2c, 7, 8, 9, 10, 12b, 15c, 15e |
| | N-(1-(4-(methylthio)benzyl)piperidin-3-yl)isoquinolin-5- | |
| | amine | |
| 2.010 | 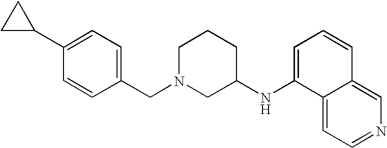 | 2c, 7, 8, 9, 10, 11, 14c |
| | N-(1-(4-cyclopropylbenzyl)piperidin-3-yl)isoquinolin-5- | |
| | amine | |
| 2.011 | 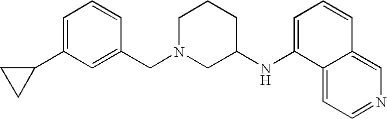 | 2c, 7, 8, 9, 10, 11, 14c |
| | N-(1-(3-cyclopropylbenzyl)piperidin-3-yl)isoquinolin-5- | |
| | amine | |
| 2.012 | 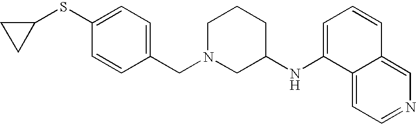 | 2c, 7, 8, 9, 10, 12b, 15c, 15e |
| | N-(1-(4-(cyclopropylthio)benzyl)piperidin-3- | |
| | yl)isoquinolin-5-amine | |
| 2.013 | 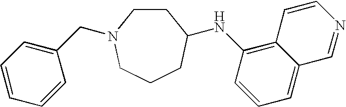 | 2c, 7, 8, 9, 10 |
| | N-(1-benzylazepan-4-yl)isoquinolin-5-amine | |
| 2.014 | 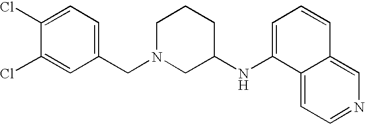 | 2c, 7, 8, 9, 10 |
| | N-(1-(3,4-dichlorobenzyl)piperidin-3-yl)isoquinolin-5- | |
| | amine | |
| 2.015 | 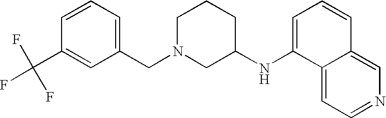 | 2c, 7, 8, 9, 10 |
| | N-(1-(3-(trifluoromethyl)benzyl)piperidin-3- | |
| | yl)isoquinolin-5-amine | |
| 2.016 | 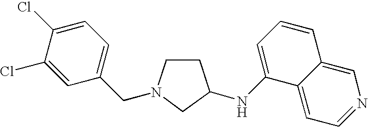 | 2c, 7, 8, 9, 10 |
| | N-(1-(3,4-dichlorobenzyl)pyrrolidin-3-yl)isoquinolin-5- | |
| | amine | |
| 2.017 | 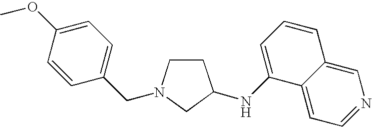 | 2c, 7, 8, 9, 10 |
| | N-(1-(4-methoxybenzyl)pyrrolidin-3-yl)isoquinolin-5- | |
| | amine | |
| 2.018 | 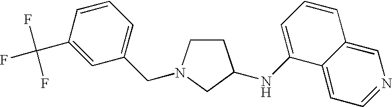 | 2c, 7, 8, 9, 10 |
| | N-(1-(3-(trifluoromethyl)benzyl)pyrrolidin-3- | |
| | yl)isoquinolin-5-amine | |
| 2.019 | 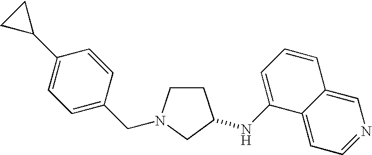 | 2c, 7, 8, 9, 10, 11, 14c |
| | (S)-N-(1-(4-cyclopropylbenzyl)pyrrolidin-3- | |
| | yl)isoquinolin-5-amine | |
| 2.020 | 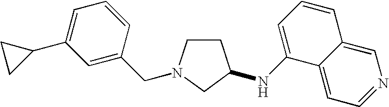 | 2c, 7, 8, 9, 10, 11, 14c |
| | (R)-N-(1-(3-cyclopropylbenzyl)pyrrolidln-3- | |
| | yl)isoquinolin-5-amine | |
| 2.021 | 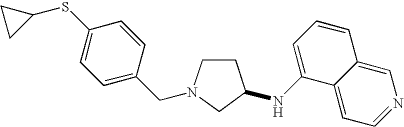 | 2c, 7, 8, 9, 10, 12b, 15c, 15e |
| | (R)-N-(1-(4-(cyclopropylthio)benzyl)pyrrolidin-3- | |
| | yl)isoguinolin-5-amine | |
| 2.022 | 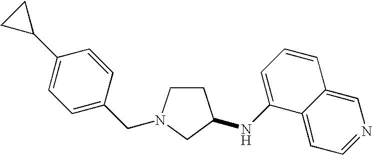 | 2c, 7, 8, 9, 10, 11, 14c |
| | (R)-N-(1-(4-cyclopropylbenzyl)pyrrolidin-3- | |
| | yl)isoquinolin-5-amine | |
| 2.023 | 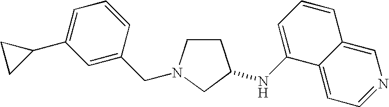 | 2c, 7, 8, 9, 10, 11, 14c |
| | (S)-N-(1-(3-cyclopropylbenzyl)pyrrolidin-3- | |
| | yl)isoquinolin-5-amine | |
| 2.024 | 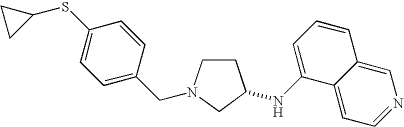 | 2c, 7, 8, 9, 10, 12b, 15c, 15e |
| | (S)-N-(1-(4-(cyclopropylthio)benzyl)pyrrolidin-3- | |
| | yl)isoquinolin-5-amine | |
| 2.025 | 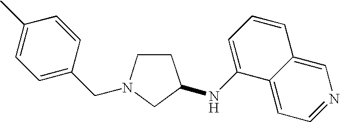 | 2c, 7, 8, 9, 10 |
| | (R)-N-(1-(4-methylbenzyl)pyrrolidin-3-yl)isoquinolin-5- | |
| | amine | |
| 2.026 | 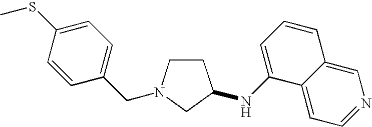 | 2c, 7, 8, 9, 10, 12b, 15c, 15e |
| | (R)-N-(1-(4-(methylthio)benzyl)pyrrolidin-3- | |
| | yl)isoquinolin-5-amine | |
| 2.027 | 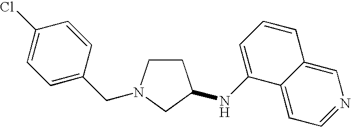 | 2c, 7, 8, 9, 10 |
| | (R)-N-(1-(4-chlorobenzyl)pyrrolidin-3-yl)isoquinolin-5- | |
| | amine | |
| 2.028 | 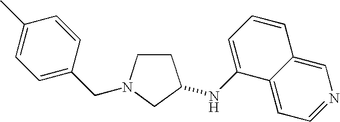 | 2c, 7, 8, 9, 10 |
| | (S)-N-(1-(4-methylbenzyl)pyrrolidin-3-yl)isoquinolin-5- | |
| | amine | |
| 2.029 | 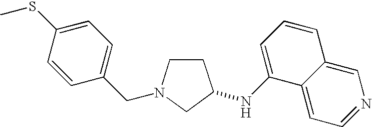 | 2c, 7, 8, 9, 10, 12b, 15c, 15e |
| | (S)-N-(1-(4-(methylthio)benzyl)pyrrolidin-3- | |
| | yl)isoquinolin-5-amine | |
| 2.030 | 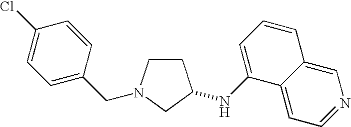 | 2c, 7, 8, 9, 10 |
| | (S)-N-(1-(4-chlorobenzyl)pyrrolidin-3-yl)isoquinolin-5- | |
| | amine | |
| 2.031 | 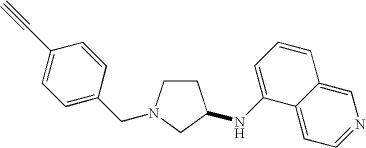 | 2c, 7, 8, 9, 10, 11, 14c |
| | (R)-N-(1-(4-ethynylbenzyl)pyrrolidin-3-yl)isoquinolin-5- | |
| | amine | |
| 2.032 | 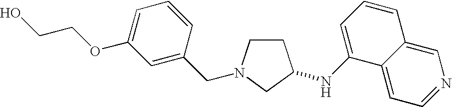 | 2c, 7, 8, 9, 10, 12a, 15c, 15d |
| | (S)-2-(3-((3-(isoquinolin-5-ylamino)pyrrolidin-1- | |
| | yl)methyl)phenoxy)ethanol | |
| 2.033 | 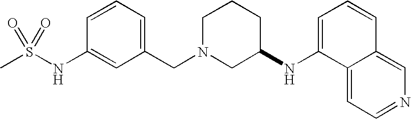 | 2c, 7, 8, 9, 10, 12a, 15c, 15d |
| | (R)-N-(3-((3-(isoquinolin-5-ylamino)piperidin-1- | |
| | yl)methyl)phenyl)methanesulfonamide | |
| 2.034 | 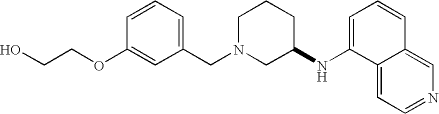 | 2c, 7, 8, 9, 10, 12a, 15c, 15d |
| | (R)-2-(3-((3-(isoquinolin-5-ylamino)piperidin-1- | |
| | yl)methyl)phenoxy)ethanol | |
| 2.035 | 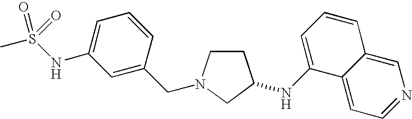 | 2c, 7, 8, 9, 10, 12a, 15c, 15d |
| | (S)-N-(3-((3-(isoquinolin-5-ylamino)pyrrolidin-1- | |
| | yl)methyl)phenyl)methanesulfonamide | |
| 2.036 | 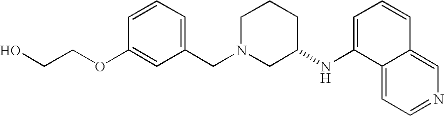 | 2c, 7, 8, 9, 10, 12a, 15c, 15d |
| | (S)-2-(3-((3-(isoquinolin-5-ylamino)piperidin-1- | |
| | yl)methyl)phenoxy)ethanol | |
| 2.037 | 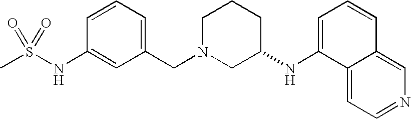 | 2c, 7, 8, 9, 10, 12a, 15c, 15d |
| | (S)-N-(3-((3-(isoquinolin-5-ylamino)piperidin-1- | |
| | yl)methyl)phenyl)methanesulfonamide | |
| 2.038 | 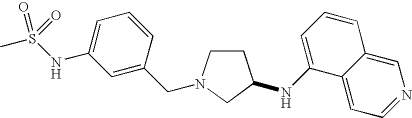 | 2c, 7, 8, 9, 10, 12a, 15c, 15d |
| | (R)-N-(3-((3-(isoquinolin-5-ylamino)pyrrolidin-1- | |
| | yl)methyl)phenyl)methanesulfonamide | |
| 2.039 | 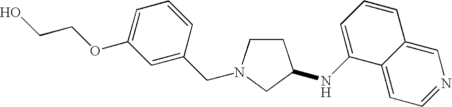 | 2c, 7, 8, 9, 10, 12a, 15c, 15d |
| | (R)-2-(3-((3-(isoquinolin-5-ylamino)pyrrolidin-1- | |
| | yl)methyl)phenoxy)ethanol | |
| 2.040 | 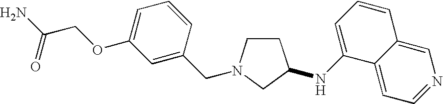 | 2c, 7, 8, 9, 10, 12a, 15c, 15d |
| | (R)-2-(3-((3-(isoquinolin-5-ylamino)pyrrolidin-1- | |
| | yl)methyl)phenoxy)acetamide | |
| 2.041 | 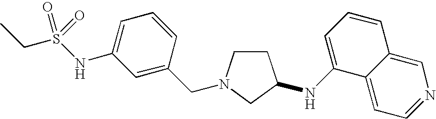 | 2c, 7, 8, 9, 10, 12a, 15c, 15d |
| | (R)-N-(3-((3-(isoquinolin-5-ylamino)pyrrolidin-1- | |
| | yl)methyl)phenyl)ethanesulfonamide | |
| 2.042 | 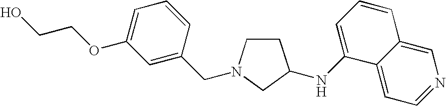 | 2c, 7, 8, 9, 10, 12a, 15c, 15d |
| | 2-(3-((3-(isoquinolin-5-ylamino)pyrrolidin-1- | |
| | yl)methyl)phenoxy)ethanol | |
| 2.043 | 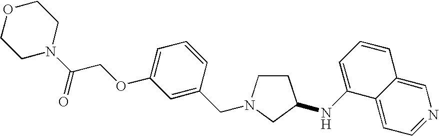 | 2c, 7, 8, 9, 10, 12a, 15c, 15d |
| | (R)-2-(3-((3-(isoquinolin-5-ylamino)pyrrolidin-1- | |
| | yl)methyl)phenoxy)-1-morpholinoethanone | |
| 2.044 | 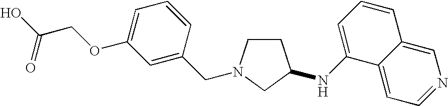 | 2c, 7, 8, 9, 10, 12a, 15c, 15d |
| | (R)-2-(3-((3-(isoquinolin-5-ylamino)pyrrolidin-1- | |
| | yl)methyl)phenoxy)acetic acid | |
| 2.045 | 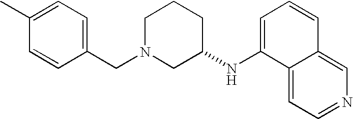 | 2c, 7, 8, 9, 10 |
| | (S)-N-(1-(4-methylbenzyl)piperidin-3-yl)isoquinolin-5- | |
| | amine | |
| 2.046 | 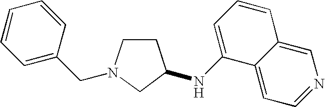 | 2c, 7, 8, 9, 10 |
| | (R)-N-(1-benzylpyrrolidin-3-yl)isoquinolin-5-amine | |
| 2.047 | 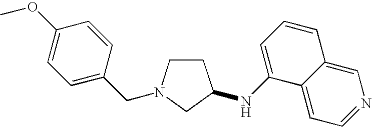 | 2c, 7, 8, 9, 10 |
| | (R)-N-(1-(4-methoxybenzyl)pyrrolidin-3-yl)isoquinolin- | |
| | 5-amine | |
| 2.048 | 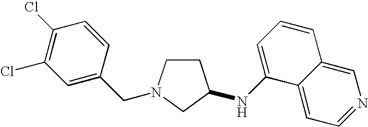 | 2c, 7, 8, 9, 10 |
| | (R)-N-(1-(3,4-dichlorobenzyl)pyrrolidin-3- | |
| | yl)isoquinolin-5-amine | |
| 2.049 | 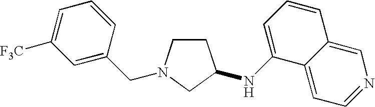 | 2c, 7, 8, 9, 10 |
| | (R)-N-(1-(3-(trifluoromethyl)benzyl)pyrrolidin-3- | |
| | yl)isoquinolin-5-amine | |
| 2.050 | 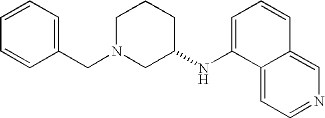 | 2c, 7, 8, 9, 10 |
| | (S)-N-(1-benzylpiperidin-3-yl)isoquinolin-5-amine | |
| 2.051 | 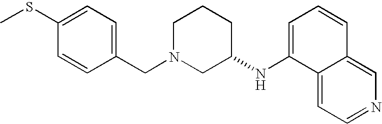 | 2c, 7, 8, 9, 10, 12b, 15c, 15e |
| | (S)-N-(1-(4-(methylthio)benzyl)piperidin-3- | |
| | yl)isoquinolin-5-amine | |
| 2.052 | 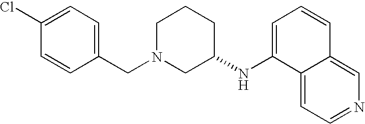 | 2c, 7, 8, 9, 10 |
| | (S)-N-(1-(4-chlorobenzyl)piperidin-3-yl)isoquinolin-5- | |
| | amine | |
| 2.053 | 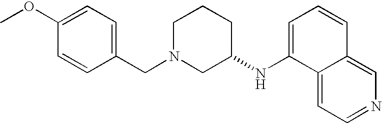 | 2c, 7, 8, 9, 10 |
| | (S)-N-(1-(4-methoxybenzyl)piperidin-3-yl)isoquinolin- | |
| | 5-amine | |
| 2.054 | 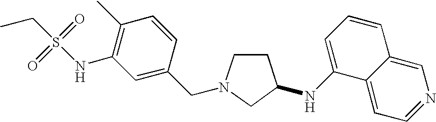 | 2c, 7, 8, 9, 10, 12a, 15c, 15d |
| | (R)-N-(5-((3-(isoquinolin-5-ylamino)pyrrolidin-1- | |
| | yl)methyl)-2-methylphenyl)ethanesulfonamide | |
| 2.055 | 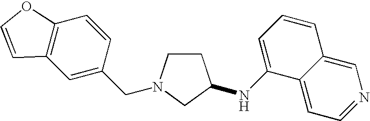 | 2c, 7, 8, 9, 10 |
| | (R)-N-(benzofuran-5-ylmethyl)pyrrolidin-3- | |
| | yl)isoquinolin-5-amine | |
| 2.056 | 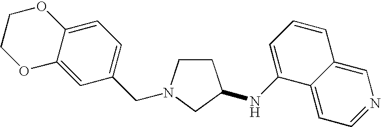 | 2c, 7, 8, 9, 10 |
| | (R)-N-(1-((2,3-dihydrobenzo[b][1,4]dioxin-6- | |
| | yl)methyl)pyrrolidin-3-yl)isoquinolin-5-amine | |
| 2.057 | 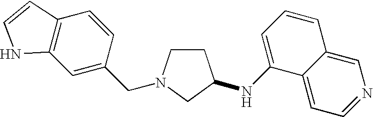 | 2c, 7, 8, 9, 10 |
| | (R)-N-(1-((1H-indol-6-yl)methyl)pyrrolidin-3- | |
| | yl)isoquinolin-5-amine | |
| 2.058 | 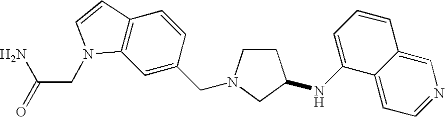 | 2c, 7, 8, 9, 10, 13, 16c |
| | (R)-2-(6-((3-(isoquinolin-5-ylamino)pyrrolidin-1- | |
| | yl)methyl)-1H-indol-1-yl)acetamide | |
| 2.059 | 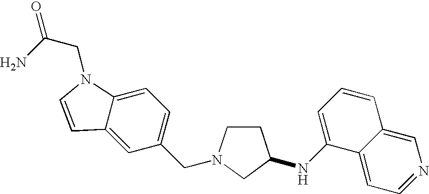 | 2c, 7, 8, 9, 10, 13, 16c |
| | (R)-2-(5-((3-(isoquinolin-5-ylamino)pyrrolidin-1- | |
| | yl)methyl)-1H-indol-1-yl)acetamide | |
| 2.060 | 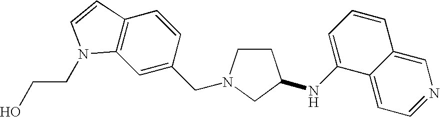 | 2c, 7, 8, 9, 10, 13, 16c |
| | (R)-2-(6-((3-(isoquinolin-5-ylamino)pyrrolidin-1- | |
| | yl)methyl)-1H-indol-1-yl)ethanol | |
| 2.061 | 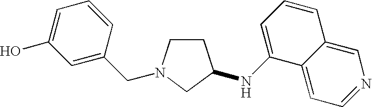 | 2c, 7, 8, 9, 10 |
| | (R)-3-((3-(isoquinolin-5-ylamino)pyrrolidin-1- | |
| | yl)methyl)phenol | |
| 2.062 | 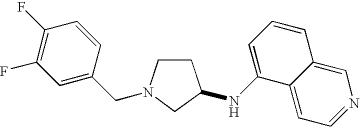 | 2c, 7, 8, 9, 10 |
| | (R)-N-(1-(3,4-difluorobenzyl)pyrrolidin-3-yl)isoquinolin- | |
| | 5-amine | |
| 2.063 | 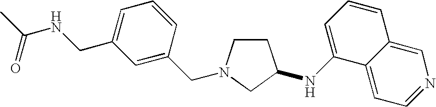 | 2c, 7, 8, 9, 10, 13, 16c |
| | (R)-N-(3-((3-(isoquinolin-5-ylamino)pyrrolidin-1- | |
| | yl)methyl)benzyl)acetamide | |
| 2.064 | 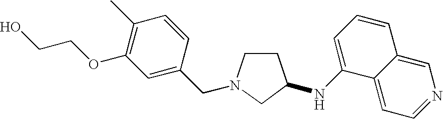 | 2c, 7, 8, 9, 10, 12a, 15c, 15d |
| | (R)-2-(5-((3-(isoquinolin-5-ylamino)pyrrolidin-1- | |
| | yl)methyl)-2-methylphenoxy)ethanol | |
| 2.065 | 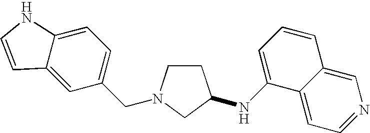 | 2c, 7, 8, 9, 10 |
| | (R)-N-(1-((1H-indol-5-yl)methyl)pyrrolidin-3- | |
| | yl)isoquinolin-5-amine | |
| 2.066 | 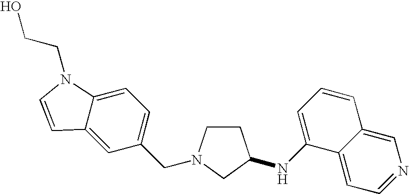 | 2c, 7, 8, 9, 10, 13, 16c |
| | (R)-2-(5-((3-(isoquinolin-5-ylamino)pyrrolidin-1- | |
| | yl)methyl)-1H-indol-1-yl)ethanol | |
| 2.067 | 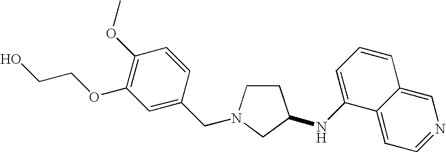 | 2c, 7, 8, 9, 10, 12a, 15c, 15d |
| | (R)-2-(5-((3-(isoquinolin-5-ylamino)pyrrolidin-1- | |
| | yl)methyl)-2-methoxyphenoxy)ethanol | |
| 2.068 | 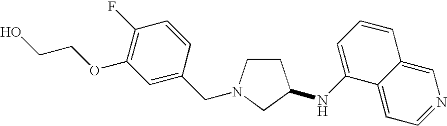 | 2c, 7, 8, 9, 10, 12a, 15c, 15d |
| | (R)-2-(2-fluoro-5-((3-(isoquinolin-5-ylamino)pyrrolidin- | |
| | 1-yl)methyl)phenoxy)ethanol | |
| 2.069 | 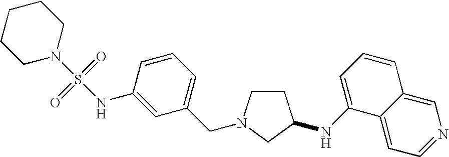 | 2c, 7, 8, 9, 10, 12a, 15c, 15d |
| | (R)-N-(3-((3-(isoquinolin-5-ylamino)pyrrolidin-1- | |
| | yl)methyl)phenyl)piperidine-1-sulfonamide | |
| 2.070 | 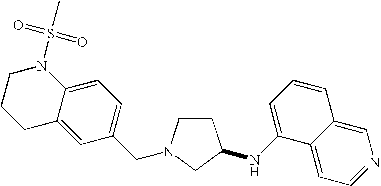 | 2c, 7, 8, 9, 10, 12b, 15c, 15d |
| | (R)-N-(1-((1-(methylsulfonyl)-1,2,3,4- | |
| | tetrahydroquinolin-6-yl)methyl)pyrrolidin-3- | |
| | yl)isoquinolin-5-amine | |
| 2.071 | 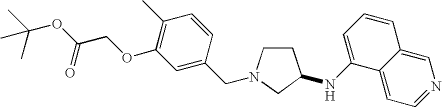 | 2c, 7, 8, 9, 10, 12a, 15c, 15d |
| | (R)-tert-butyl 2-(5-((3-(isoquinolin-5-ylarnino)pyrrolidin- | |
| | 1-yl)methyl)-2-methylphenoxy)acetate | |
| 2.072 | 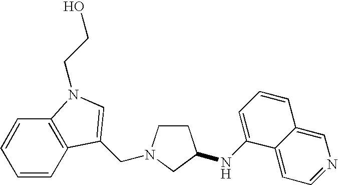 | 2c, 7, 8, 9, 10, 13, 16c |
| | (R)-2-(3-((3-(isoquinolin-5-ylamino)pyrrolidin-1- | |
| | yl)methyl)-1H-indol-1-yl)ethanol | |
| 2.073 | 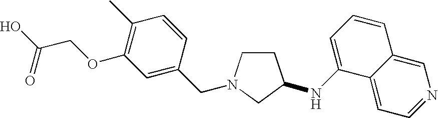 | 2c, 7, 8, 9, 10, 12a, 15c, 15d |
| | (R)-2-(5-((3-(isoquinolin-5-ylamino)pyrrolidin-1- | |
| | yl)methyl)-2-methylphenoxy)acetic acid | |
| 2.074 | 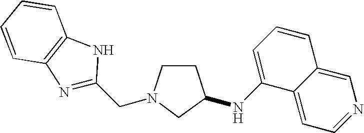 | 2c, 7, 8, 9, 10 |
| | (R)-N-(1-((1H-benzo[c]imidazol-2-yl)methyl)pyrrolidin- | |
| | 3-yl)isoquinolin-5-amine | |
| 2.075 | 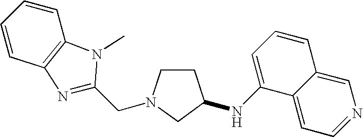 | 2c, 7, 8, 9, 10 |
| | (R)-N-(1-((1-methyl-1H-benzo[d]imidazol-2- | |
| | yl)methyl)pyrrolidin-3-yl)isoquinolin-5-amine | |
| 2.076 | 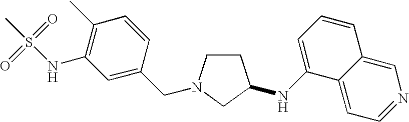 | 2c, 7, 8, 9, 10, 12a, 15c, 15d |
| | (R)-N-(5-((3-(isoquinolin-5-ylamino)pyrrolidin-1- | |
| | yl)methyl)-2-methylphenyl)methanesulfonamide | |
| 2.077 | 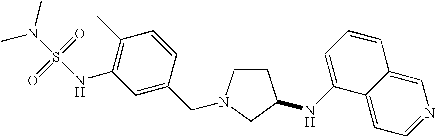 | 2c, 7, 8, 9, 10, 12a, 15c, 15d |
| | (R)-N-(5-((3-(isoquinolin-5-ylamino)pyrrolidin-1- | |
| | yl)methyl)-2-methylphenyl)-N′,N′ | |
| | dimethylaminosulfamide | |
| 2.078 | 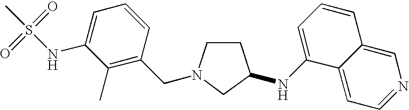 | 2c, 7, 8, 9, 10, 12a, 15c, 15d |
| | (R)-N-(3-((3-(isoquinolin-5-ylamino)pyrrolidin-1- | |
| | yl)methyl)-2-methylphenyl)methanesulfonamide | |
| 2.079 | 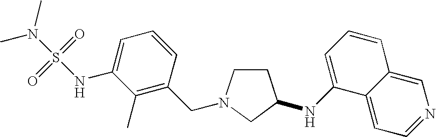 | 2c, 7, 8, 9, 10, 12a, 15c, 15d |
| | (R)-N-(3-((3-(isoquinolin-5-ylamino)pyrrolidin-1- | |
| | yl)methyl)-2-methylphenyl)-N′,N′ | |
| | dimethylaminosulfamide | |
| 2.080 | 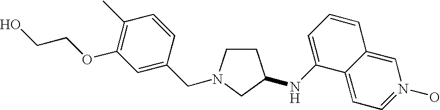 | 2b, 6b, 8, 9, 10, 12a, 15b, 15d |
| | (R)-5-(1-(3-(2-hydroxyethoxy)-4- | |
| | methylbenzyl)pyrrolidin-3-ylamino)isoquinoline 2-oxide | |
| 2.081 | 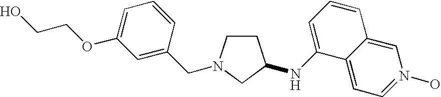 | 2b, 6b, 8, 9, 10, 12a, 15b, 15d |
| | (R)-5-(1-(3-(2-hydroxyethoxy)benzyl)pyrrolidin-3- | |
| | ylamino)isoquinoline 2-oxide | |
| 2.082 | 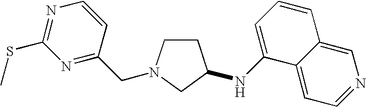 | 2c, 7, 8, 9, 10, 12b, 15c, 15e |
| | (R)-N-(1-((2-(methylthio)pyrimidin-4- | |
| | yl)methyl)pyrrolidin-3-yl)isoquinolin-5-amine | |
| 2.083 | 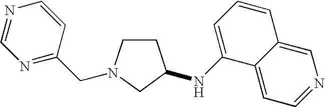 | 2c, 7, 8, 9, 10 |
| | (R)-N-(1-(pyrimidin-4-ylmethyl)pyrrolidin-3- | |
| | yl)isoquinolin-5-amine | |
| 2.084 | 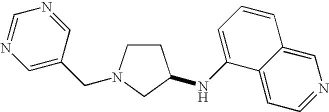 | 2c, 7, 8, 9, 10 |
| | (R)-N-(1-(pyrimidin-5-ylmethyl)pyrrolidin-3- | |
| | yl)isoquinolin-5-amine | |
| 2.085 | 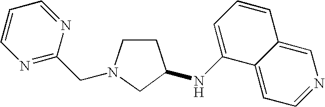 | 2c, 7, 8, 9, 10 |
| | (R)-N-(1-(pyrimidin-2-ylmethyl)pyrrolidin-3- | |
| | yl)isoquinolin-5-amine | |
| 2.086 | 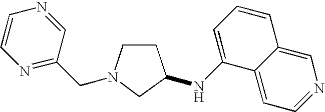 | 2c, 7, 8, 9, 10 |
| | (R)-N-(1-(pyrazin-2-ylmethyl)pyrrolidin-3- | |
| | yl)isoquinolin-5-amine | |
| 2.087 | 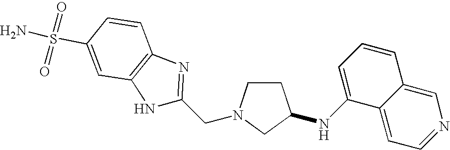 | 2c, 7, 8, 9, 10, 12b, 15c, 15e |
| | (R)-2-((3-(isoquinolin-5-ylamino)pyrrolidin-1- | |
| | yl)methyl)-1H-benzo[d]imidazole-6-sulfonamide | |
| 2.088 | 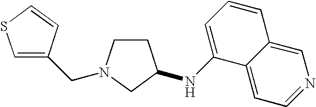 | 2c, 7, 8, 9, 10 |
| | (R)-N-(1-(thiophen-3-ylmethyl)pyrrolidin-3- | |
| | yl)isoquinolin-5-amine | |
| 2.089 | 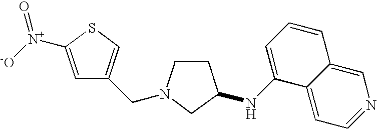 | 2c, 7, 8, 9, 10 |
| | (R)-N-(1-((5-nitrothiophen-3-yl)methyl)pyrrolidin-3- | |
| | yl)isoquinoIin-5-amine | |
| 2.090 | 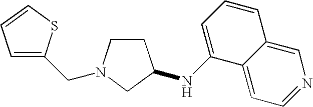 | 2c, 7, 8, 9, 10 |
| | (R)-N-(1-(thiophen-2-ylmethyl)pyrrolidin-3- | |
| | yl)isoguinolin-5-amine | |
| 2.091 | 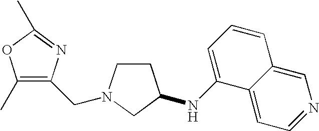 | 2c, 7, 8, 9, 10 |
| | (R)-N-(1-((2,5-dimethyloxazol-4-yl)methyl)pyrrolidin-3- | |
| | yl)isoquinolin-5-amine | |
| 2.092 | 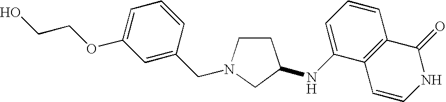 | 2b, 6b, 8, 9,10, 12a, 15b, 15d |
| | (R)-5-(1-(3-(2-hydroxyethoxy)benzyl)pyrrolidin-3- | |
| | ylamino)isoquinolin-1(2H)-one | |
| 2.093 | 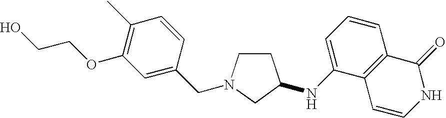 | 2b, 6b, 8, 9, 10, 12a, 15b, 15d |
| | (R)-5-(1-(3-(2-hydroxyethoxy)-4- | |
| | methylbenzyl)pyrrolidin-3-ylamino)isoquinolin-1(2H)- | |
| | one | |
| 2.094 | 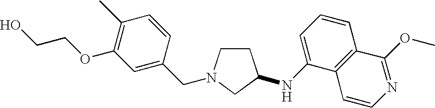 | 2b, 6b, 8, 9, 10, 12a, 15b, 15d |
| | (R)-2-(5-((3-(1-methoxylsoquinolin-5- | |
| | ylamino)pyrrolidin-1-yl)methyl)-2- | |
| | methylphenoxy)ethanol | |
| 2.095 | 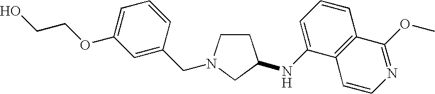 | 2b, 6b, 8, 9, 10, 12a, 15b, 15d |
| | (R)-2-(3-((3-(1-methoxylsoquinolin-5- | |
| | ylamino)pyrrolidin-1-yl)methyl)phenoxy)ethanol | |
| 2.096 | 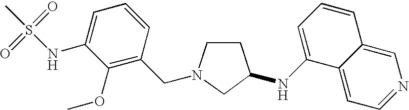 | 2c, 7, 8, 9, 10, 12a, 15c, 15d |
| | (R)-N-(3-((3-(isoquinolin-5-ylamino)pyrrolidin-1- | |
| | yl)methyl)-2-methoxyphenyl)methanesulfonamide | |
| 2.097 | 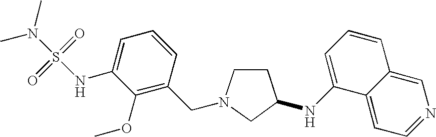 | 2c, 7, 8, 9, 10, 12a, 15c, 15d |
| | (R)-N-(3-((3-(isoquinolin-5-ylamino)pyrrolidin-1- | |
| | yl)methyl)-2-methoxyphenyl)-N′,N′ | |
| | dimethylaminosulfamide | |
| 2.098 | 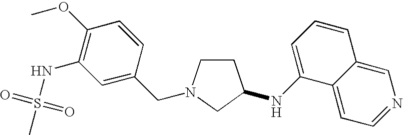 | 2c, 7, 8, 9, 10, 12a, 15c, 15d |
| | (R)-N-(5-((3-(isoquinolin-5-ylamino)pyrrolidin-1- | |
| | yl)methyl)-2-methoxyphenyl)methanesulfonamide | |
| 2.099 | 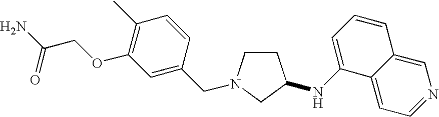 | 2c, 7, 8, 9, 10, 12a, 15c, 15d |
| | (R)-2-(5-((3-(isoquinolin-5-ylamino)pyrrolidin-1- | |
| | yl)methyl)-2-methylphenoxy)acetamide | |
| 2.100 | 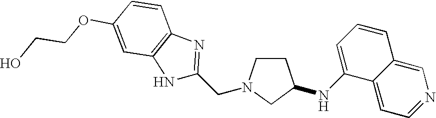 | 2c, 7, 8, 9, 10, 12a, 15c, 15d |
| | (R)-2-(2-((3-(isoquinolin-5-ylamino)pyrrolidin-1- | |
| | yl)methyl)-1H-benzo[d]imidazol-6-yloxy)ethanol | |
| 3.001 | 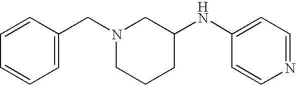 | 3c, 7, 8, 9, 10 |
| | N-(1-benzylpiperidin-3-yl)pyridin-4-amine | |
| 3.002 | 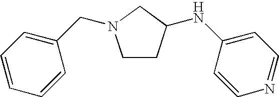 | 3c, 7, 8, 9, 10 |
| | N-(1-benzylpyrrolidin-3-yl)pyridin-4-amine | |
| 4.001 | 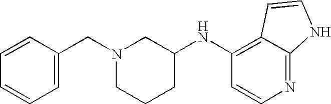 | 4c, 7, 8, 9, 10 |
| | N-(1-benzylpiperidin-3-yl)-1H-pyrrolo[2,3-b]pyridin-4- | |
| | amine | |
| 4.002 | 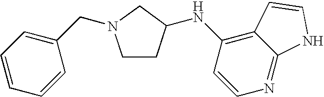 | 4c, 7, 8, 9, 10 |
| | N-(1-benzylpyrrolidin-3-yl)-1H-pyrrolo[2,3-b]pyridin-4- | |
| | amine | |
| 5.001 | 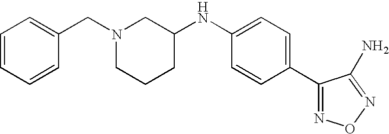 | 5a, 7, 8, 9, 10 |
| | 4-(4-(1-benzylpiperidin-3-ylamino)phenyl)-1,2,5- | |
| | oxadiazol-3-amine | |
| 5.002 | 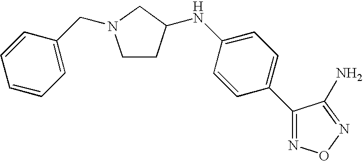 | 5a, 7, 8, 9, 10 |
| | 4-(4-(1-benzylpyrrolidin-3-ylamino)phenyl)-1,2,5- | |
| | oxadiazol-3-amine | |
| | 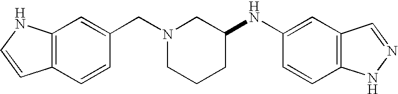 | |
| | (S)-N-(1-((1H-indol-6-yl)methyl)piperidin-3-yl)-1H- | |
| | indazol-5-amine | |

Preferred ROCK inhibitor compounds useful for this invention include the ROCK inhibitor compounds of embodiments 5, 14, 15, 16, 17, 18, 19, 20, and 21 as described above, and their associated salts, tautomers, solvates, or hydrates. In particular, preferred Compounds include 1.074, 1.075, 1.076, 1.077, 1.079, 1.091, 1.093, 1.108, 1.109, 1.123, 1.124, 1.126, 1.131, 1.132, 1.133, 1.134, 1.135, 1.136, 1.137, 1.138, 1.141, 1.148, 1.149, 1.150, 1.152, 1.153, 1.155, 1.156, 1.157, 1.158, 1.161, 1.162, 1.163, 1.164, 1.165, 1.166, 1.171, 1.173, 1.175, 1.176, 1.186, 1.193, 1.195, 1.197, 1.200, 1.206, 1.212, 1.213, 1.215, 1.217, 1.219, 1.223, 1.233, 1.236, 1.237, 1.238, 1.239, 1.249, 1.252, 1.253, 1.258, 1.259, 1.260, 1.261, 1.262, 1.270, 1.273, 1.275, 1.277, 1.281, 2.025, 2.026, 2.031, 2.038, 2.039, 2.041, 2.046, 2.047, 2.054, 2.055, 2.057, 2.058, 2.059, 2.060, 2.061, 2.064, 2.065, 2.066, 2.067, 2.068, 2.069, 2.072, 2.073, 2.076, 2.077, 2.078, 2.079, 2.082, 2.096, 2.097, and 2.099.

More preferred compounds are 1.074, 1.075, 1.091, 1.107, 1.123, 1.124, 1.152, 1.153, 1.161, 1.162, 1.165, 1.197, 1.212, 1.213, 1.215, 1.076, 1.077, 1.093, 1.106, 1.108, 1.109, 1.127, 1.157, 1.158, 1.159, 1.176, 1.185, 1.186, 1.195, 1.2, 1.206, 1.208, 1.217, 1.219, 1.223, 1.229, 1.233, 1.236, 1.237, 1.238, 1.239, 1.249, 1.253, 2.058, 2.059, 2.06, 2.066, 1.258, and 1.262.

### Pharmaceutical Formulations

The Rho kinase inhibitor compounds can be formulated in a pharmaceutically acceptable carrier. Pharmaceutically acceptable carriers can be selected by those skilled in the art using conventional criteria. Pharmaceutically acceptable carriers include, but are not limited to, saline solution, aqueous electrolyte solutions, isotonicity modifiers, water polyethers such as polyethylene glycol, polyvinyls such as polyvinyl alcohol and povidone, cellulose derivatives such as methylcellulose and hydroxypropyl methylcellulose, polymers of acrylic acid such as carboxypolymethylene gel, polysaccharides such as dextrans, and glycosaminoglycans such as sodium hyaluronate and salts such as sodium chloride and potassium chloride.

The pharmaceutical formulation useful for the present invention in general is preferably an aqueous solution comprising water, suitable ionic or non-ionic tonicity modifiers, suitable buffering agents, and a Rho kinase inhibitor compound. In one embodiment, the compound is at 0.005 to 3% w/v, and the aqueous solution has a tonicity of 200-400 mOsm/kG and a pH of 4-9.

In one embodiment of this invention, the tonicity modifier is ionic such as NaCl, for example, in the amount of 0.5-0.9% w/v, preferably 0.6-0.9% w/v.

In another embodiment of this invention, the tonicity modifier is non-ionic, such as mannitol, dextrose, in the amount of at least 2%, or at least 2.5%, or at least 3%, and no more than 7.5%; for example, in the range of 3-5%, preferably 4-5% w/v.

The pharmaceutical formulation can be sterilized by filtering the formulation through a sterilizing grade filter, preferably of a 0.22-micron nominal pore size. The pharmaceutical formulation can also be sterilized by terminal sterilization using one or more sterilization techniques including but not limited to a thermal process, such as an autoclaving process, or a radiation sterilization process, or using pulsed light to produce a sterile formulation. In one embodiment, the pharmaceutical formulation is a concentrated solution of the active ingredient; the formulation can be serially diluted using appropriate acceptable sterile diluents prior to administration.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents such as those set forth above, and flavoring agents can be added to provide palatable oral preparations. These compositions can be preserved by the addition of an anti-oxidant such as ascorbic acid.

Pharmaceutical compositions of the invention can be in the form of oil-in-water emulsions. The oily phase can be a vegetable oil, for example olive oil or arachis oil, or a mineral oil, for example liquid paraffin or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions can also contain sweetening and flavoring agents.

Pharmaceutical compositions of the invention can be in the form of an aerosol suspension of respirable particles comprising the active compound, which the subject inhales. The respirable particles can be liquid or solid, with a particle size sufficiently small to pass through the mouth and larynx upon inhalation. In general, particles having a size of about 1 to 10 microns, preferably 1 to 5 microns, are considered respirable.

The pharmaceutical formulation for systemic administration such as injection and infusion is generally prepared in a sterile medium. The active ingredient, depending on the vehicle and concentration used, can either be suspended or dissolved in the vehicle. Adjuvants such as local anesthetics, preservatives and buffering agents can also be dissolved in the vehicle. The sterile injectable preparation can be a sterile injectable solution or suspension in a non-toxic acceptable diluent or solvent. Among the acceptable vehicles and solvents that can be employed are sterile water, saline solution, or Ringer's solution.

The pharmaceutical compositions for oral administration contain active compounds in the form of tablets, lozenges, aqueous or oily suspensions, viscous gels, chewable gums, dispersible powders or granules, emulsion, hard or soft capsules, or syrups or elixirs.

For oral use, an aqueous suspension is prepared by addition of water to dispersible powders and granules with a dispersing or wetting agent, suspending agent, one or more preservatives, and other excipients. Suspending agents include, for example, sodium carboxymethylcellulose, methylcellulose and sodium alginate. Dispersing or wetting agents include naturally-occurring phosphatides, condensation products of an allylene oxide with fatty acids, condensation products of ethylene oxide with long chain aliphatic alcohols, condensation products of ethylene oxide with partial esters from fatty acids and a hexitol, and condensation products of ethylene oxide with partial esters derived from fatty acids and hexitol anydrides. Preservatives include, for example, ethyl, and n-propyl p-hydroxybenzoate. Other excipients include sweetening agents (e.g., sucrose, saccharin), flavoring agents and coloring agents. Those skilled in the art will recognize the many specific excipients and wetting agents encompassed by the general description above.

For oral application, tablets are prepared by mixing the active compound with nontoxic pharmaceutically acceptable excipients suitable for the manufacture of tablets. These excipients can be, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets can be uncoated or they can be coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil. Formulation for oral use can also be presented as chewable gums by embedding the active ingredient in gums so that the active ingredient is slowly released upon chewing.

The pharmaceutical compositions can be in the form of suppositories, which are prepared by mixing the active ingredient with a suitable non-irritating excipient that is solid at ordinary temperatures but liquid at the rectal temperature and will thus melt in the rectum to release the compound. Such excipients include cocoa butter and polyethylene glycols.

### Method of Treating Pulmonary Diseases Using Rho Kinase Inhibitor Compounds

The present invention is useful in treating patients with pulmonary diseases associated with bronchoconstriction or inflammation and who have no significant response to treatment with beta adrenergic receptor agonists or the combined treatment with beta adrenergic receptor agonists and corticosteroids. In a preferred embodiment, the present invention is useful for treating patients with asthma, COPD, respiratory tract illness caused by respiratory syncytial virus infection such as RSV-induced wheezing, airway hyperreactivity, or bronchiolitis, who have reduced responsiveness to treatment with beta adrenergic receptor agonists or the combined treatment with beta adrenergic receptor agonists and corticosteroids.

The present invention is also useful for treating pulmonary diseases in patients who had reduced responsiveness to treatment with beta adrenergic receptor agonists but has regained responsiveness to the beta adrenergic receptor agonist after a the combined treatment with the beta adrenergic receptor agonist and a corticosteroid.

The present methods comprise the steps of first identifying a patient that fits within the above-described subset of patient population, and then administering to the patient an effective amount of a Rho kinase inhibitor compound.

### Asthma

A method for treating asthma in patients who have reduced responsiveness to treatment with beta adrenergic receptor agonist or the combined treatment with beta adrenergic receptor agonists and corticosteroids is based on the properties of Rho kinase inhibitors to demonstrate efficacy as bronchorelaxants under conditions where there is reduced responsiveness to beta adrenergic receptor agonists either due to desensitization or due to increased pro-inflammatory cytokines in steroid resistant pulmonary disease states.

Indicia of efficacy for treating asthma by the present method include demonstrable improvement in measurable signs, symptoms and other variables clinically relevant to asthma. Such improvements include increased blood oxygen saturation, decreased hypoxia and hypercapnia, decrease need for supplemental oxygen, decreased frequency of coughing and/or wheezing, improved forced expiratory volume (FEV₁), forced vital capacity (FVC) or other physiologically relevant parameter of respiratory function, decrease in nighttime awakenings, decrease in interference with normal activity, decrease need to use short-acting beta agonist for symptom relief, an improved score in standard questionnaires such as the Asthma Therapy Assessment Questionnaire (ATAQ), Asthma Control Questionnaire (ACQ) or Asthma Control Test (ACT) as defined in EPR-3 asthma guidelines, decreased need for mechanical ventilation, lower amount of inflammatory cells infiltrating the lung, lower levels of pro-inflammatory cytokines and chemokines, improved alveolar fluid clearance rate, decreased pulmonary edema as determined by any radiographic or other detection method such as amount of epithelial lining fluid, wet to dry lung weight, alveolar fluid clearance and/or radiographic visualization methods, increase in general quality of life, the levels of inflammatory cells in the lung or outside of the lung in other anatomical compartments or spaces including systemic circulation, the amount of pro-inflammatory molecules including cytokines and chemokines in the lung or outside of the lung in other anatomical compartments or spaces including systemic circulation, pathological remodeling of the airway, patient-reported or physician-observed signs such as ease of breathing, or severity of coughing and/or wheezing.

### COPD

A method for treating chronic obstructive pulmonary disease in patients who have reduced responsiveness to treatment with beta adrenergic receptor agonist or the combined treatment with beta adrenergic receptor agonists and corticosteroids is based on the properties of Rho kinase inhibitors to demonstrate efficacy as bronchorelaxants under conditions where there is reduced responsiveness to beta adrenergic receptor agonists either due to desensitization or due to increased pro-inflammatory cytokines in steroid resistant pulmonary disease states.

Indicia of efficacy for treating COPD by the present method include demonstrable improvement in measurable signs, symptoms and other variables clinically relevant to COPD. Such improvements include decreased frequency of exacerbations, increased blood oxygen saturation, decreased hypoxia and hypercapnia, decrease need for supplemental oxygen, decreased frequency of coughing and/or wheezing, improved forced expiratory volume (FEV₁), forced vital capacity (FVC) or other physiologically relevant parameter of respiratory function, decreased need for mechanical ventilation, lower amount of inflammatory cells infiltrating the lung, lower levels of pro-inflammatory cytokines and chemokines, improved alveolar fluid clearance rate, decreased pulmonary edema as determined by any radiographic or other detection method such as amount of epithelial lining fluid, wet to dry lung weight, alveolar fluid clearance or radiographic visualization methods, increase in general quality of life, the levels of inflammatory cells in the lung or outside of the lung in other anatomical compartments or spaces including systemic circulation, the amount of pro-inflammatory molecules including cytokines and chemokines in the lung or outside of the lung in other anatomical compartments or spaces including systemic circulation, pathological remodeling of the airway, patient-reported or physician-observed signs such as ease of breathing, or severity of coughing and/or wheezing.

### Respiratory Tract Illness Caused by Respiratory Syncytial Virus Infection Such as RSV-Induced Wheezing, Airway Hyperreactivity, or Bronchiolitis

A method for treating respiratory tract illness caused by respiratory syncytial virus infection such as RSV-induced wheezing, airway hyperreactivity, or bronchiolitis in patients who have reduced responsiveness to treatment with beta adrenergic receptor agonist or the combined treatment with beta adrenergic receptor agonists and corticosteroids is based on the properties of Rho kinase inhibitors to demonstrate efficacy as bronchorelaxants under conditions where there is reduced responsiveness to beta adrenergic receptor agonists either due to desensitization or due to increased pro-inflammatory cytokines in steroid resistant pulmonary disease states.

Indicia of efficacy for treating respiratory tract illness caused by respiratory syncytial virus infection such as RSV-induced wheezing, airway hyperreactivity, or bronchiolitis by the present method include demonstrable improvement in measurable signs, symptoms and other variables clinically relevant to respiratory tract illness caused by respiratory syncytial virus infection such as RSV-induced wheezing and hyperreactivity or bronchiolitis.

Such improvements include decreased frequency of exacerbations, increased blood oxygen saturation, decreased hypoxia and hypercapnia, decrease need for supplemental oxygen, decreased frequency of coughing and/or wheezing, improved forced expiratory volume (FEV1), forced vital capacity (FVC) or other physiologically relevant parameter of respiratory function, decreased need for mechanical ventilation, lower amount of inflammatory cells infiltrating the lung, lower levels of proinflammatory cytokines and chemokines, improved alveolar fluid clearance rate, decreased pulmonary edema as determined by any radiographic or other detection method such as amount of epithelial lining fluid, wet to dry lung weight, alveolar fluid clearance or radiographic visualization methods, increase in general quality of life, the levels of inflammatory cells in the lung or outside of the lung in other anatomical compartments or spaces including systemic circulation, the amount of pro-inflammatory molecules including cytokines and chemokines in the lung or outside of the lung in other anatomical compartments or spaces including systemic circulation, pathological remodeling of the airway, patient-reported or physician-observed signs such as ease of breathing, or severity of coughing and/or wheezing.

### Bronchiectasis, Alpha-1-Antitrypsin Deficiency (AATD), Lymphangioleiomyomatosis (LAM), Cystic Fibrosis, Bronchiolitis/Wheezing, Chronic Bronchitis, and Occupational Lung Diseases, Byssinosis, Asbestosis and Silicosis

A method for treating bronchiectasis, alpha-1-antitrypsin deficiency (AATD), lymphangioleiomyomatosis (LAM), cystic fibrosis, bronchiolitis or wheezing caused by agents other than respiratory syncytial virus, chronic bronchitis, and occupational lung diseases such as coal workers' pneumoconiosis, byssinosis (brown lung disease), asbestosis and silicosis in patients who have reduced responsiveness to treatment with beta adrenergic receptor agonist or the combined treatment with beta adrenergic receptor agonists and corticosteroids is based on the properties of Rho kinase inhibitors to demonstrate efficacy as bronchorelaxants under conditions where there is reduced responsiveness to beta adrenergic receptor agonists either due to desensitization or due to increased pro-inflammatory cytokines in steroid resistant pulmonary disease states.

Indicia of efficacy for treating bronchiectasis, alpha-1-antitrypsin deficiency (AATD), lymphangioleiomyomatosis (LAM), cystic fibrosis, bronchiolitis/wheezing, chronic bronchitis, and occupational lung diseases such as coal workers' pneumoconiosis, byssinosis (brown lung disease), asbestosis and silicosis by the present method include demonstrable improvement in measurable signs, symptoms and other variables clinically relevant to bronchiectasis, alpha-1-antitrypsin deficiency (AATD), lymphangioleiomyomatosis (LAM), cystic fibrosis, bronchiolitis/wheezing, chronic bronchitis, or occupational lung diseases such as coal workers' pneumoconiosis, byssinosis (brown lung disease), asbestosis and silicosis. Such improvements include decreased frequency of exacerbations, increased blood oxygen saturation, decreased hypoxia and hypercapnia, decrease need for supplemental oxygen, decreased frequency of coughing and/or wheezing, improved forced expiratory volume (FEV₁), forced vital capacity (FVC) or other physiologically relevant parameter of respiratory function, decreased need for mechanical ventilation, lower amount of inflammatory cells infiltrating the lung, lower levels of pro-inflammatory cytokines and chemokines, improved alveolar fluid clearance rate, decreased pulmonary edema as determined by any radiographic or other detection method such as amount of epithelial lining fluid, wet to dry lung weight, alveolar fluid clearance or radiographic visualization methods, increase in general quality of life, the levels of inflammatory cells in the lung or outside of the lung in other anatomical compartments or spaces including systemic circulation, the amount of pro-inflammatory molecules including cytokines and chemokines in the lung or outside of the lung in other anatomical compartments or spaces including systemic circulation, pathological remodeling of the airway, patient-reported or physician-observed signs such as ease of breathing, or severity of coughing and/or wheezing.

### Methods of Administration

The present invention provides a method for treating pulmonary disease such as asthma, COPD, respiratory tract illness caused by respiratory syncytial virus infection such as RSV-induced wheezing, airway hyperreactivity, or bronchiolitis, bronchiectasis, alpha-1-antitrypsin deficiency (AATD), lymphangioleiomyomatosis (LAM), cystic fibrosis, bronchiolitis or wheezing caused by agents other than respiratory syncytial virus, chronic bronchitis, or occupational lung diseases such as coal workers' pneumoconiosis, byssinosis (brown lung disease), asbestosis and silicosis. Any method of delivering the compound to the relevant tissues of the lung, including local administration and systemic administration, is suitable for the present invention.

In a preferred embodiment, the active compound is delivered by local administration to the lung. Local administration includes inhalation, topical application or targeted drug delivery. Methods of inhalation include liquid instillation, instillation as a pressurized fluid preparation via metered dose inhaler or equivalent, or inhalation of an aerosolized solution via nebulizer, inhalation of dry powder, and directing soluble or dried material into the air stream during mechanical ventilation.

One local administration method is administering to a subject an aerosol suspension of respirable particles comprising the active compound by inhalation. The respirable particles can be liquid or solid, with a particle size sufficiently small to pass through the mouth and larynx upon inhalation; in general, particles ranging from about 1 to 10 microns, but more preferably 1 to 5 microns, in size are considered respirable. The surface concentrations of active compounds delivered via inhalation can vary according to compounds; but are generally 1×10⁻¹⁰1×10⁻⁴ moles/liter, and preferably 1×10⁻⁸−1×10⁻⁵ moles/liter.

An example of targeted drug delivery is enclosure of the compound within a liposome, where the liposome is coated with a specific antibody whose antigen is expressed in the targeted lung tissue. It can be advantageous to construe a controlled delivery system of the compounds since such an inhaled product targets the site of action, presents the compound of interest in small regimented quantities and reduces/minimizes any unwanted side effects.

Another example of a delivery system includes microparticulate compositions of the compound. In such a case, the compound is formulated as a microparticulate wherein the carrier is loaded with the compound; such a preparation is then filtered through a fine porous membrane or suitable filtering medium or is exposed to solvent interchanges to produce nanoparticles. Such preparations can be freeze dried or held in suspension in an aqueous or physiologically compatible medium. The preparation so obtained can be inhaled by suitable means.

Another example of a suitable preparation includes a reconstitutable preparation. In this case, the compound is formulated in a preparation to contain the necessary adjuvant to make it physiologically compatible. Such a preparation can be reconstituted by addition of water for injection or suitable physiological fluids, admixed by simple agitation and inhaled using appropriate techniques described above.

The compounds described above can also be prepared into dry powder or equivalent inhalation powders using the well known art of super critical fluid technology. In such a case, the compound is admixed with appropriate excipients and milled into a homogenous mass using suitable solvents or adjuvants. Following this, this mass is subjected to mixing using super critical fluid technology and suitable particle size distribution achieved. The particles in the formulation need to be of a desired particle size range such that the particles can be directly inhaled into the lungs using a suitable inhalation technique or introduced into the lungs via a mechanical ventilator. Alternatively, a formulation can be designed such that the particles are large enough in size thereby offering sufficient surface area to dissolve completely in a suitable fluid when admixed together or to dissolve sufficiently enough prior to nebulization into the lungs.

To prevent particle size growth and minimize crystal growth of the compound, one embodiment is to include the use of spray-dried particles that have better aerodynamic properties than micronized material. This can be further extended to coat the surface of the hydrophilic molecule with one or more layers of hydrophobic material

In another embodiment, the active compound is delivered by systemic administration; the compound first reaches plasma and then distributes into the lung tissues. Examples of systemic administration include oral ingestion, intravenous, subcutaneous, intraperitoneal, or intramuscular administration.

Additional method of systemic administration of the active compound to the lungs of a subject involves administering a suppository form of the active compound, such that a therapeutically effective amount of the compound reaches the target sites via systemic absorption and circulation.

Another method of systemically administering the active compounds to the lungs of the subject involves administering a liquid/liquid suspension in the form of nasal drops of a liquid formulation. Liquid pharmaceutical compositions of the active compound for producing a nasal drop can be prepared by combining the active compound with a suitable vehicle, such as sterile pyrogen free water or sterile saline by techniques known to those skilled in the art.

The active compounds can also be systemically administered to the lungs of the subject through absorption by the skin using transdermal patches or pads. The active compounds are absorbed into the bloodstream through the skin. Plasma concentration of the active compounds can be controlled by using patches containing different concentrations of active compounds.

For systemic administration, plasma concentrations of active compounds delivered can vary according to compounds; but are generally 1×10⁻¹⁰1×10⁻⁴ moles/liter, and preferably 1×10⁻⁸−1×10⁻⁵ moles/liter.

Dosage levels about 0.01-140 mg per kg of body weight per day are useful in the treatment or prevention of pulmonary diseases (about 0.5 mg to about 7 g per patient per day). Preferred dosage levels are about 0.05-100, 0.1-100, or 1-100 mg/kg body weight per day. The amount of active ingredient that can be combined with the carrier materials to produce a single dosage form will vary depending upon the host treated and the particular mode of administration. Dosage unit forms will generally contain between from about 1 mg to about 500 mg of an active ingredient.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more can be administered to achieve adequate steady state levels. The maximum total dose in general does not exceed about 2 g/day for a 40 to 80 kg human patient.

Frequency of dosage can also vary depending on the compound used and the particular disease treated. However, for treatment of most disorders, a dosage regimen of p.r.n, 4 times daily, three times daily, or less is preferred, with a dosage regimen of once daily or 2 times daily being particularly preferred.

It is understood, however, that the specific dose level for any particular patient will depend upon a variety of factors including the activity of the specific compound employed, the age, body weight, general health, sex, diet, time of administration, route of administration, and rate of excretion, drug combination (i.e., other drugs being administered to the patient), the severity of the particular disease undergoing therapy, and other factors, including the judgment of the prescribing medical practitioner.

Preferred compounds of the invention will have favorable pharmacological properties. Such properties include but are not limited to bioavailability, low toxicity, low serum protein binding and desirable in vitro and in vivo half-life.

Assays can be used to predict these desirable pharmacological properties. Assays used to predict bioavailability include transport across human intestinal cell monolayers, including Caco-2 cell monolayers. Toxicity to cultured hepatocycles can be used to predict compound toxicity.

The invention is illustrated further by the following examples that are not to be construed as limiting the invention in scope to the specific procedures described in them.

### EXAMPLES

### Example 1

### Efficacy of Rho Kinase Inhibitors in Tracheal Smooth Muscle with Reduced Responsiveness to Beta Adrenergic Receptor Agonist

### Protocol

Trachea were excised from male Sprague-Dawley rats, cleaned of connective tissue and cut into cylindrical segments of 2-3 mm length. Two stainless steel wires were guided through the lumen of the tracheal ring. One wire was fixed in the tissue bath and the other was connected to a force transducer via surgical silk. Preparations were mounted in 5 ml water-jacketed organ baths (Radnoti Glass Technology) filled with Krebs buffer (95 mM NaCl, 5 mM KCl, 2.6 mM CaCl₂, 1.2 mM MgSO₄, 24.9 mM NaHCO₃, 1.2 mM KH₂PO₄, 10 mM glucose) maintained at 37° C. and gassed with 95% O₂ and 5% CO₂. Indomethacin (1 μM), a cyclooxygenase inhibitor, was added to the Krebs buffer and was present throughout the experiments. Contractile tensions were measured using an isometric force transducer (Grass Instruments) and signals were analyzed using specialized software (Chart v5.5, ADInstruments). The preparations were allowed to equilibrate at a resting tension of 0.3 to 0.5 gm prior to two challenges with 60 mM KCl to assess tissue viability. After washing, tissues were treated with 300 nM carbachol for 10 to 15 minutes to induce a contractile response. Tissues were then treated with isoproterenol, formoterol, or albuterol to induce a relaxant response. However, this relaxant response is transient such that formoterol, albuterol or isoproterenol lose efficacy over time and a fade of the response toward a more contractile state occurs in the continued presence of formoterol, albuterol or isoproterenol. After 10 minutes of formoterol treatment, these tissues were treated with a second dose of formoterol or with albuterol. After an additional 10 minutes, tissues were treated with a Rho kinase inhibitor. Basal tension was subtracted from all values and data were reported as a percentage of the maximal carbachol-induced contraction. Four parameters were analyzed for quantitation of efficacies: i) the maximal relaxation induced by initial formoterol treatment, ii) the fade of the response to the initial formoterol treatment, iii) the maximal relaxation induced by the second addition of a beta adrenergic receptor agonist, and iv) the maximal relaxation induced by Rho kinase inhibitor.

### Results

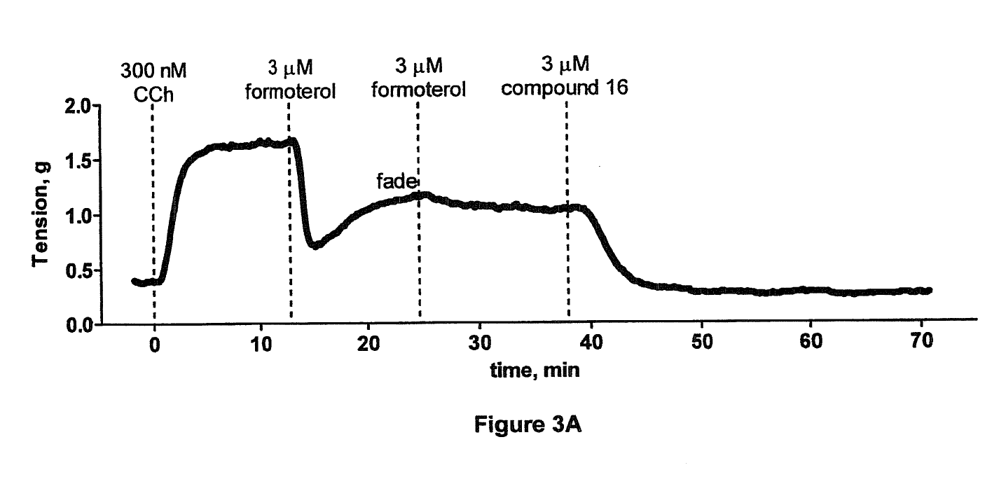

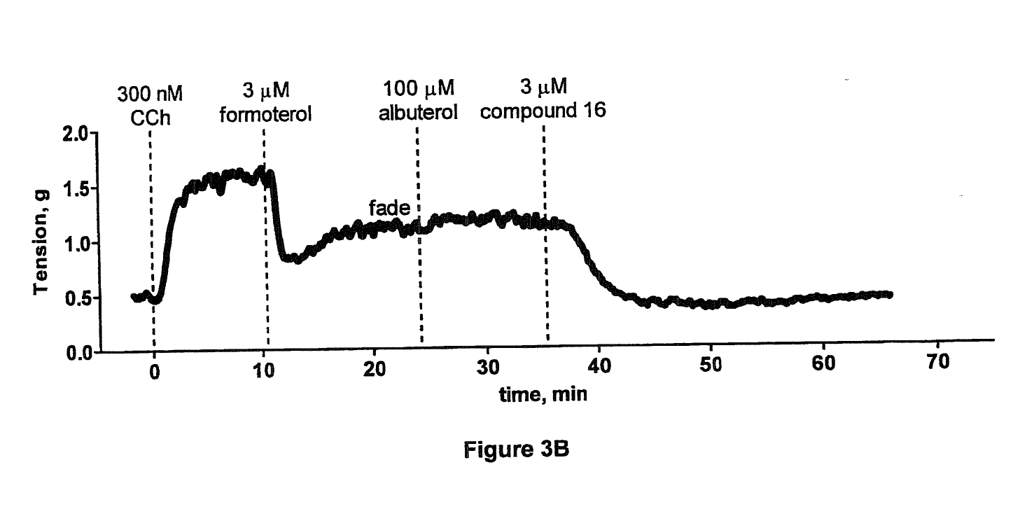

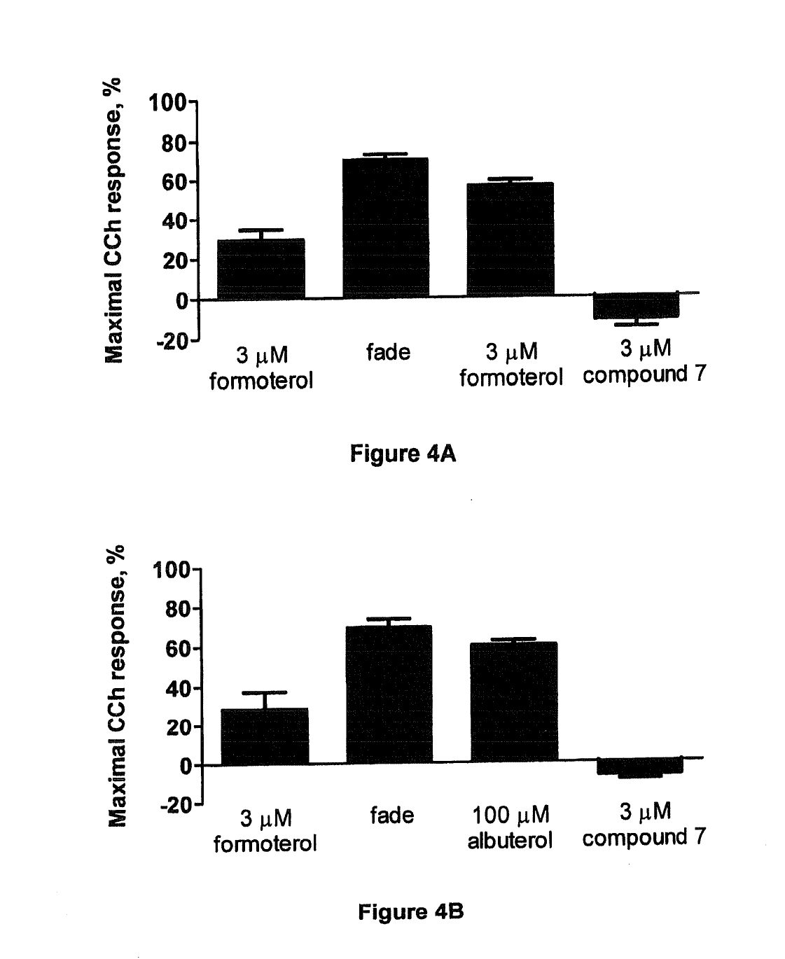

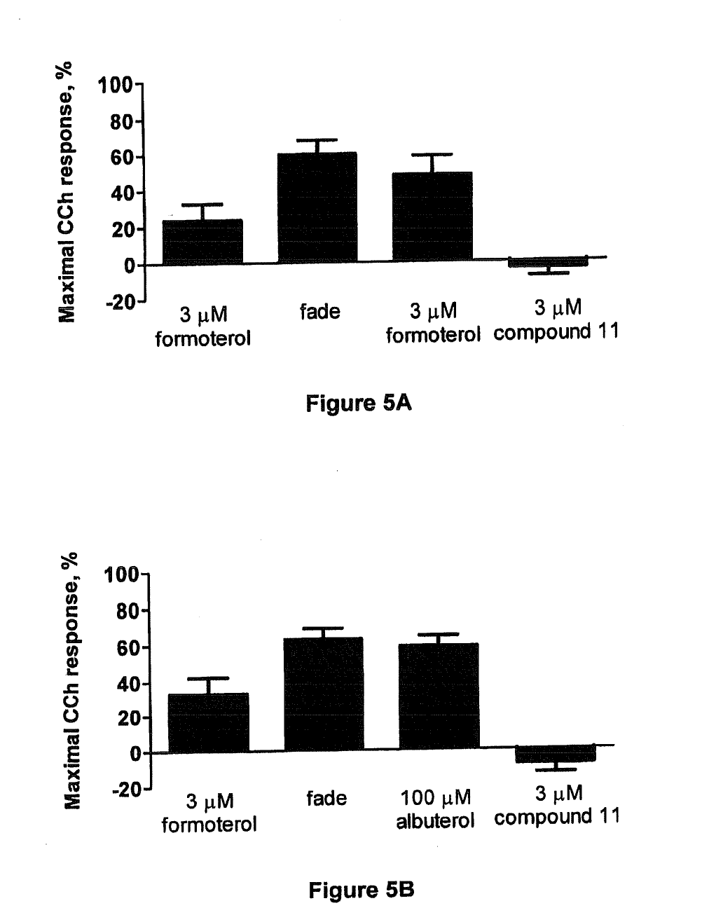

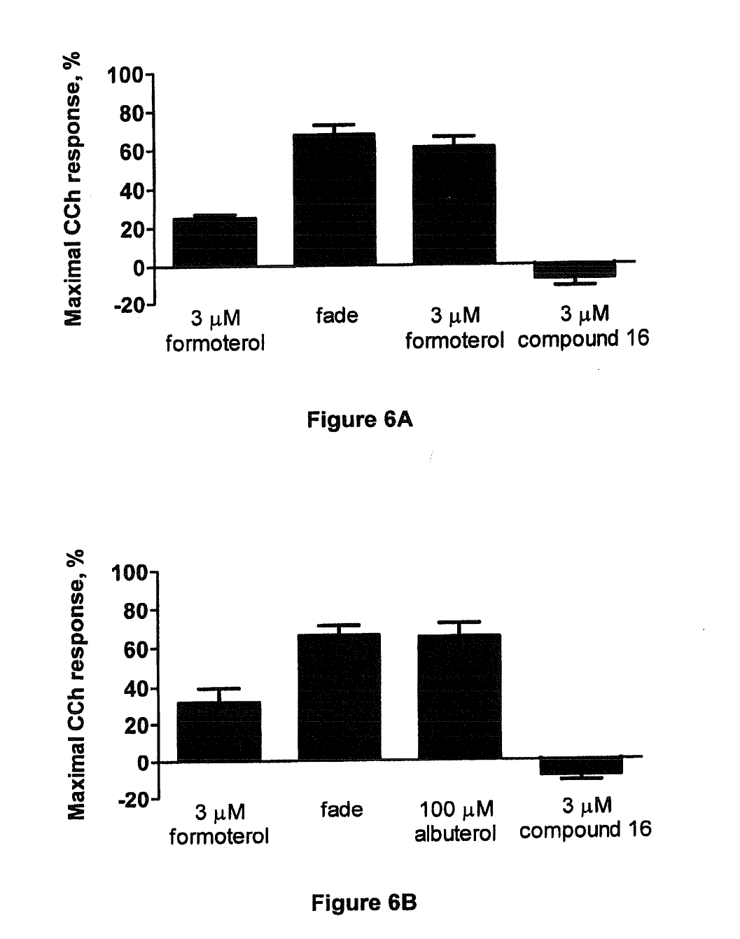

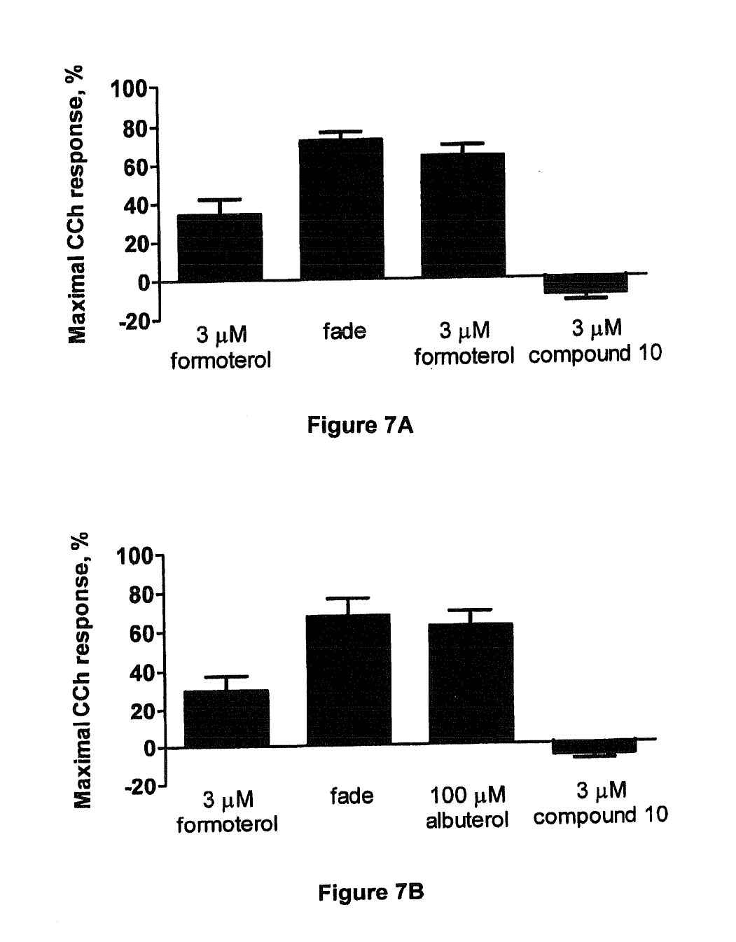

Application of 1 μM isoproterenol, 1 μM formoterol or 30 μM albuterol to carbachol-precontracted trachea resulted in an initial rapid and partial relaxation of the tissue followed by a slower fade of the response to a more contractile state (FIG. 1A-C). In contrast, application of 3 μM Rho kinase inhibitor compounds to carbachol-precontracted trachea resulted in a sustained relaxation of tracheal preparations to basal tension (FIG. 1D-E). The dose of each compound was confirmed to be a maximally effective dose under these conditions (data not shown). These data demonstrate a marked contrast in the longevity of response to beta adrenergic receptor agonist versus Rho kinase inhibitor compounds. Under conditions where the response to 3 μM formoterol has faded, application of a second dose of 3 μM formoterol (FIG. 2A) or 100 μM albuterol (FIG. 2B) was ineffective in relaxing the tracheal preparation. Under these conditions of reduced responsiveness to beta adrenergic receptor agonist, 3 μM compound 7 was fully efficacious in relaxing the tracheal preparation (FIG. 2A-B). Similarly, the representative traces in FIG. 3 demonstrate that under conditions where the response to 3 μM formoterol has faded, application of a second dose of 3 μM formoterol (FIG. 3A) or 100 μM albuterol (FIG. 3B) was ineffective in relaxing the tracheal preparation. Under these conditions of reduced responsiveness to beta adrenergic receptor agonist, 3 μM compound 16 was fully efficacious in relaxing the tracheal preparation (FIG. 3A-B). These representative traces demonstrate that both indazole based Rho kinase inhibitors (exemplified by compound 7) and isoquinoline based Rho kinase inhibitors (exemplified by compound 16) are fully efficacious in relaxing tracheal preparation that shows reduced responsiveness to treatment with beta adrenergic receptor agonists. Quantitation of these effects across multiple experiments is shown in FIGS. 4-7. In each set of experiments, 3 μM formoterol induces a relaxant response that is approximately 30% of the initial carbachol response. This initial relaxation was lost and followed by a fade of the response in the continued presence of formoterol such that the contraction was 60% to 70% of the initial carbachol response (FIGS. 4-7). As shown in FIG. 4-7, addition of a second dose of 3 μM formoterol or of 100 μM albuterol was ineffective in relaxing the tissue indicating that the tissue has reduced responsiveness to treatment with beta agonists. Subsequent addition of 3 μM compound 7, an indazole-based Rho kinase inhibitor compound, to the tissue resulted in a complete relaxation of the tissue (FIG. 4A-B). Subsequent addition of 3 μM compound 11, an isoquinoline-based Rho kinase inhibitor compound, to the tissue resulted in a complete relaxation of the tissue (FIG. 5A-B). Subsequent addition of 3 μM compound 16, an indazole-based Rho kinase inhibitor compound, to the tissue resulted in a complete relaxation of the tissue (FIG. 6A-B). Subsequent addition of 3 μM compound 10, an isoquinoline-based Rho kinase inhibitor compound, to the tissue resulted in a complete relaxation of the tissue (FIG. 7A-B). The ability of multiple Rho kinase inhibitors to induce a relaxant response in tissue that has reduced responsiveness to beta-adrenergic receptor agonist demonstrates that Rho kinase inhibitors as a class are effective in reducing smooth muscle tone and contractibility after the induction of tolerance (desensitization) to beta adrenergic receptor agonists.

### Example 2

### Efficacy of Rho Kinase Inhibitors in Tracheal Smooth Muscle with Reduced Responsiveness to Beta Adrenergic Receptor Agonist Due to Pretreatment with Pro-Inflammatory Cytokines

### Relevance

Pulmonary disease such as asthma and COPD are accompanied by an inflammatory response in the lung that contributes to disease severity. In patients with corticosteroid resistant asthma and COPD, increased levels of TNFalpha and IL-1beta have been shown. These pro-inflammatory cytokines can alter tissue function and may limit the efficacy of therapeutic interventions such beta adrenergic receptor agonists. In vitro demonstration of compound efficacy in tissues that have been exposed to pro-inflammatory cytokines supports the utility of these compounds as bronchorelaxants in patients who have reduced responsiveness to treatment with beta adrenergic receptor agonist or the combined treatment with beta adrenergic receptor agonists and corticosteroids.

### Protocol

Male Sprague-Dawley rats weighing 301-325 gm were sacrificed by asphyxiation in a CO₂ chamber. Trachea were excised, cleaned of connective tissue and cut into cylindrical segments of 2-3 mm length. Tissues were treated for 18 hours at 37° C. in F12 media with penicillin-streptomycin and 0.1% BSA alone or with 10 ng/ml IL-1β and 100 ng/ml TNF-α. IL-1β and TNF-α are pro-inflammatory cytokines. Tissues were then washed free of cytokines with Krebs buffer. Contractile tensions were measured using an isometric force transducer (Grass Instruments) as described for Example 1 and signals were analyzed using specialized software (Chart v5.5, ADInstruments). Tissues were treated with 300 nM carbachol for 10 to 15 minutes to induce a contractile response. Beta adrenergic receptor agonist compounds, isoproterenol or albuterol, were added cumulatively to the bath every 2 to 3 minutes and reductions in tension were recorded. Alternatively, Rho kinase inhibitor compound was added cumulatively to the bath every 30 minutes and reductions in tension were recorded. Basal tension was subtracted from all values and data were reported as a percentage of the maximal carbachol-induced contraction. Data were fit to the Hill equation using GraphPad Prism v5 software.

FIG. 8 and FIG. 9 show the dose response relationship for isoproterenol and albuterol, respectively, to induce a relaxant response in vehicle-pretreated or cytokine-pretreated tissues. The IC₅₀ for isoproterenol from vehicle-pretreated and cytokine-pretreated tissue is 33 nM and 71 nM, respectively. The IC₅₀ for salbutamol from vehicle-pretreated and cytokine-pretreated tissue is 239 nM and 411 nM, respectively. These data show that pretreatment with pro-inflammatory cytokines reduces the efficacy and potency of both beta adrenergic receptor agonists. FIG. 10 shows the dose response relationship for compound 7 to induce a relaxant response in vehicle-pretreated or cytokine-pretreated tissues. Compound 7 is fully efficacious in relaxing tracheal rings from both vehicle-pretreated and cytokine-pretreated tissues and is slightly more potent in cytokine-pretreated tissues. The IC₅₀ for compound 7 from vehicle-pretreated and cytokine-pretreated tissue is 51 nM and 28 nM, respectively. These data show that pretreatment with pro-inflammatory cytokines does not affect the efficacy and potency of Rho kinase inhibitor compounds.

### Example 3

### Pulmonary Function Test in Human Patients Treated with Formoterol Protocol

Patients with asthma or COPD are randomized to albuterol or Rho kinase inhibitor compound test groups. After 2-weeks of run-in period, subjects are given a methacholine provocation test (MPT) to induce bronchoconstriction followed by treatment with increasing doses of albuterol or with increasing doses of Rho kinase inhibitor compound to induce bronchorelaxation to establish the subject's baseline response to albuterol or Rho kinase inhibitor compound. Subjects from both test groups are then randomized to inhaled formoterol twice daily or placebo for 2 weeks. At the end of the trial period, the albuterol test group subjects are again administered a methacholine provocation test to induce bronchoconstriction followed by treatment with increasing doses of albuterol. Similarly, the Rho kinase inhibitor compound test group subjects are again administered a methacholine provocation test to induce bronchoconstriction followed by treatment with increasing doses of Rho kinase inhibitor compound to induce bronchorelaxation. The change in FEV1 after albuterol inhalation or administration of Rho kinase inhibitor compound is measured.

### Results

A decrease in the bronchodilator response to albuterol as measured by FEV1 is demonstrated in the formoterol group compared to the placebo group. In contrast to the decrease in bronchodilator response to albuterol, there is less change in bronchodilator response to Rho kinase inhibitor compounds as measured by FEV1 in the formoterol group compared to the placebo group. Patients treated with formoterol who remain symptomatic after treatment with albuterol are treated with a Rho kinase inhibitor compound and display a bronchodilator response as measured by FEV1.

### Example 4

### Treatment of Human Patients

Patients with asthma, COPD, or respiratory tract illness caused by respiratory syncytial virus infection such as RSV-induced wheezing, airway hyperreactivity, or bronchiolitis, who remain symptomatic despite high doses of beta adrenergic receptor agonist alone or in combination with a corticosteroid are administered a Rho kinase inhibitor compound, which is delivered into the lumen of their lung in the amounts ranging from 0.001 to 100 mg. Alternatively, patients suffering from asthma, COPD or respiratory tract illness caused by respiratory syncytial virus infection such as RSV-induced wheezing and hyperreactivity or bronchiolitis who remain symptomatic despite high doses of beta adrenergic receptor agonist either alone or in combination with a corticosteroid are administered a Rho kinase inhibitor compound that is delivered systemically in the amounts ranging from 0.01 to 100 mg/kg of patient's body weight.

It is observed that the administration of a Rho kinase inhibitor compound improves the health status of the patient as measured by improvement in at least one of the following measurable signs, symptoms and other variables clinically relevant to asthma, COPD or respiratory tract illness caused by respiratory syncytial virus infection such as RSV-induced wheezing, airway hyperreactivity, or bronchiolitis. Such improvements include increased blood oxygen saturation, decrease in exacerbations, decreased hypoxia and hypercapnia, decrease need for supplemental oxygen, decreased frequency of coughing and/or wheezing, improved forced expiratory volume (FEV₁), forced vital capacity (FVC) or other physiologically relevant parameter of respiratory function, decreased need for mechanical ventilation, decreased pulmonary edema, patient-reported or physician-observed signs such as ease of breathing, or severity of coughing and/or wheezing.

Although the invention has been described with reference to the presently preferred embodiments, it should be understood that various modifications could be made without departing from the scope of the invention.

What is claimed:

1. A method for treating pulmonary diseases in patients who have reduced responsiveness to treatment with one or more beta adrenergic receptor agonists, comprising the steps of:
identifying a patient who suffers from a pulmonary disease and has reduced responsiveness to treatment with one or more beta adrenergic receptor agonists, and
administering to the patient an effective amount of a Rho kinase inhibitor compound, wherein said pulmonary disease is selected from the group consisting of: asthma, chronic obstructive pulmonary disease, respiratory tract illness caused by respiratory syncytial virus infection, bronchiectasis, alpha-1-antitrypsin deficiency, lymphangioleiomyomatosis, cystic fibrosis, bronchiolitis or wheezing caused by agents other than respiratory syncytial virus, chronic bronchitis, and occupational lung diseases.

2. The method according to claim 1, wherein said reduced responsiveness is due to desensitization developed in the patient to the treatment of the beta adrenergic receptor agonists.

3. The method according to claim 1, wherein said reduced responsiveness is due to pulmonary inflammation.

4. The method according to claim 1, wherein said reduced responsiveness is due to viral infection, bacterial infection, allergen exposure, corticosteroid resistance leading to uncontrolled inflammation, treatment with beta adrenergic receptor antagonists, workplace exposure to sensitizing chemicals, environmental exposure to irritants such as tobacco smoke, or sulfite sensitivity.

5. The method according to claim 1, wherein said patient has reduced responsiveness to the combined treatment of corticosteroid and one or more beta adrenergic receptor agonists.

6. The method according to claim 1, wherein said pulmonary disease is asthma or chronic obstructive pulmonary disease.

7. The method according to claim 1, wherein said administering is local administering of the Rho kinase inhibitor compound to the lung of the patient.

8. The method according to claim 1, wherein said Rho kinase inhibitor compound is a compound of Formula II: 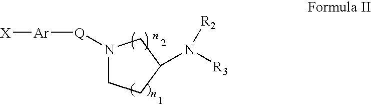
wherein:
Q is C═O, SO₂, or (CR₄R₅)ₙ₃;
n₁ is 1, 2, or 3;
n₂ is 1 or 2;
n₃ is 0, 1, 2, or 3;
wherein the ring represented by 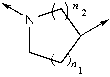
is optionally substituted by alkyl, halo, oxo, OR₆, NR₆R₇, or SR₆;
R₂ is selected from the following heteroaryl systems, optionally substituted: 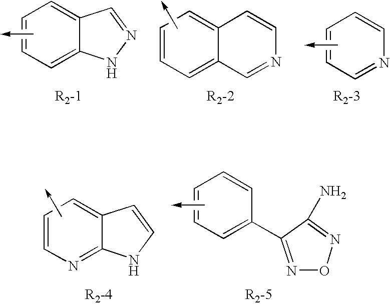
Ar is a monocyclic or bicyclic aryl or heteroaryl ring;
Y is one or more substituents on Z, and each is chosen independently from H, halogen, OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀;
Each instance of Z is chosen independently from alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, heterocycle, (heterocycle)alkyl, (heterocycle)alkenyl, (heterocycle)alkynyl, or is absent;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle, optionally substituted by one or more halogen or heteroatom-containing substituents;
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈-R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle;
wherein any two of the groups R₈, R₉ and R₁₀ are optionally joined with a link selected from the group consisting of bond, —O—, —S—, —SO—, —SO₂—, and NR₁₇— to form a ring.

9. The method according to claim 8, wherein R₂ is R₂-1 or R₂-2.

10. The method according to claim 8, wherein said compound of Formula II is a compound of Formula IIa, IIb, or IIc: 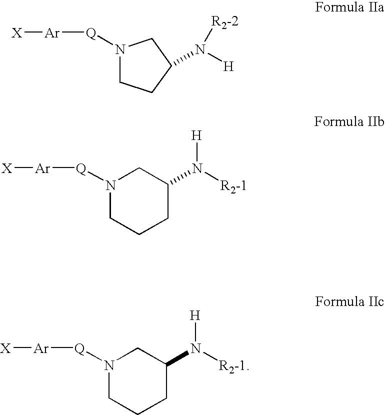
wherein Ar is phenyl, a 6,5-fused bicyclic heteroaryl ring, or a 6,6-fused bicyclic heteroaryl ring; Ar is substituted by 1 or 2 substituents X, and Q is CH₂.

11. The method according to claim 10, wherein Ar is 3-substituted phenyl; 4-substituted phenyl; 3,4-disubstituted phenyl; or 2,3-disubstituted phenyl.

12. The method according to claim 10, wherein Ar is benzofuran, benzothiophene, indole, and benzimidazole.

13. The method according to claim 8, wherein Y is H, halogen, OR₈, SR₈, SOR₈, SO₂R₈, SO₂NR₈R₉, NR₈SO₂R₉, CONR₈R₉, or NR₈C(═O)NR₉R₁₀; Z is alkyl, alkenyl, alkynyl, cycloalkyl, cycloalkenyl, cycloalkylalkyl, or is absent; Q is (CR₄R₅)ₙ₃; and n₃ is 1 or 2.

14. The method according to claim 8, wherein R₃-R₇ are H; R₈ is H, alkyl, arylalkyl, cycloalkyl, cycloalkylalkyl, or heterocycle, optionally substituted with halogen, OR₁₁, NR₁₁R₁₂, SR₁₁, SOR₁₁, SO₂R₁₁, SO₂NR₁₁R₁₂, NR₁₁SO₂R₁₂, CONR₁₁R₁₂, or NR₁₁C(═O)R₁₂; and R₉-R₁₇ are H or alkyl.

15. The method according to claim 8, wherein said compound is Compound 1.074, which is (R)-N-(1-(4-(methylthio)benzyl)piperidin-3-yl)-1H-indazol-5-amine; Compound 1.075, which is (S)-N-(1-(4-(methylthio)benzyl)piperidin-3-yl)-1H-indazol-5-amine; Compound 1.091, which is (S)-N-(3-((3-(1H-indazol-5-ylamino)piperidin-1-yl)methyl)phenyl)methanesulfonamide; Compound 1.093, which is (R)-N-(3-((3-(1H-indazol-5-ylamino)piperidin-1-yl)methyl)phenyl)methanesulfonamide; Compound 1.123, which is (R)-N-(3-((3-(1H-indazol-5-ylamino)piperidin-1-yl)methyl)phenyl)ethanesulfonamide; Compound 1.124, which is (S)-N-(3-((3-(1H-indazol-5-ylamino)piperidin-1-yl)methyl)phenyl)ethanesulfonamide; Compound 1.126, which is (R)-2-(3-((3-(1H-indazol-5-ylamino)piperidin-1-yl)methyl)phenoxy)-N-(pyridin-3-yl)acetamide; Compound 1.152, which is (S)-2-(5-((3-(1H-indazol-5-ylamino)piperidin-1-yl)methyl)-2-methylphenoxy)ethanol; Compound 1.157, which is (S)-N-(1-(3-(methylsulfonylmethyl)benzyl)piperidin-3-yl)-1H-indazol-5-amine; Compound 1.158, which is (S)-N-(1-(3-(methylthio)benzyl)piperidin-3-yl)-1H-indazol-5-amine; Compound 1.161, which is (R)-2-(5-((3-(1H-indazol-5-ylamino)piperidin-1-yl)methyl)-2-methylphenoxy)ethanol; Compound 1.195, which is (S)-2-(3-((3-(1H-indazol-5-ylamino)piperidin-1-yl)methyl)phenoxy)acetamide; Compound 1.200, which is (S)-ethyl 2-(3-((3-(1H-indazol-5-ylamino)piperidin-1-yl)methyl)phenoxy)acetate; Compound 1.212, which is (R)-N-(5-((3-(1H-indazol-5-ylamino)piperidin-1-yl)methyl)-2-chlorophenyl)methanesulfonamide; Compound 1.213, which is (S)-N-(5-((3-(1H-indazol-5-ylamino)piperidin-1-yl)methyl)-2-chlorophenyl)methanesulfonamide; Compound 1.215, which is (S)-3-((3-(1H-indazol-5-ylamino)piperidin-1-yl)methyl)benzenesulfonamide; Compound 1.219, which is (S)-3-((3-(1H-indazol-5-ylamino)piperidin-1-yl)methyl)benzamide; Compound 1.233, which is (S)-N-(5-((3-(1H-indazol-5-ylamino)piperidin-1-yl)methyl)-2-methylphenypmethanesulfonamide; Compound 1.236, which is (S)-N-(5-((3-(1H-indazol-5-ylamino)piperidin-1-yl)methyl)-2-methylphenyl)butane-1-sulfonamide; Compound 1.237, which is (S)-N-(2-((3-(1H-indazol-5-ylamino)piperidin-1-yl)methyl)-5-methylphenyl)-N′,N′dimethylaminosulfamide; Compound 1.238, which is (S)-N-(5-((3-(1H-indazol-5-ylamino)piperidin-1-yl)methyl)-2-methylphenyl)propane-1-sulfonamide; Compound 1.239, which is (S)-N-(5-((3-(1H-indazol-5-ylamino)piperidin-1-yl)methyl)-2-methylphenyl)-4-methylbenzenesulfonamide; Compound 1.249, which is (R)-3-((3-(1H-indazol-5-ylamino)piperidin-1-yl)methyl)benzenesulfonamide; Compound 1.253, which is (S)-N-(5-((3-(1H-indazol-5-ylamino)piperidin-1-yl)methyl)-2-methylphenyl)ethanesulfonamide; Compound 1.258, which is (R)-N-(5-((3-(1H-indazol-5-ylamino)piperidin-1-yl)methyl)-2-methylphenyl)methanesulfonamide; Compound 1.259, which is (R)-N-(5-((3-(1H-indazol-5-ylamino)piperidin-1-yl)methyl)-2-methylphenyl)ethanesulfonamide; Compound 1.260, which is (R)-N-(5((3-(1H-indazol-5-ylamino)piperidin-1-yl)methyl)-2-methylphenyl)-4-methylbenzenesulfonamide; Compound 1.261, which is (S)-N-(3-((3-(1H-indazol-5-ylamino)piperidin-1-yl)methyl)phenyl)-N′,N′dimethylaminosulfamide; Compound 1.262, which is (R)-N-(2-((3-(1H-indazol-5-ylamino)piperidin-1-yl)methyl)-5-methylphenyl)-N′,N′dimethylaminosulfamide; Compound 1.270, which is (S)-N-(3-((3-(1H-indazol-5-ylamino)piperidin-1-yl)methyl)phenyl)piperidine-1-sulfonamide; Compound 1.275, which is (S)-N-(3-((3-(1H-indazol-5-ylamino)piperidin-1-yl)methyl)-2-methylphenyl)-N′,N′dimethylaminosulfamide; Compound 1.281, which is (R)-2-(5-((3-(1H-indazol-5-ylamino)piperidin-1-yl)methyl)-2-methylphenyl1H-indazol-5-ylamino)piperidin-1-yl)methyl)-2-methylphenoxy)acetamide; Compound 2.026, which is (R)-N-(1-(4-(methylthio)benzyl)pyrrolidin-3-yl)isoquinolin-5-amine; Compound 2.038, which is (R)-N-(3-((3-(isoquinolin-5-ylamino)pyrrolidin-1-yl)methyl)phenyl)methanesulfonamide; Compound 2.039, which is (R)-2-(3-((3-(isoquinolin-5-ylamino)pyrrolidin-1-yl)methyl)phenoxy)ethanol; Compound 2.041, which is (R)-N-(3-((3-(isoquinolin-5-ylamino)pyrrolidin-1-yl)methyl)phenyl)ethanesulfonamide; Compound 2.054, which is (R)-N-(5-((3-(isoquinolin-5-ylamino)pyrrolidin-1-yl)methyl)-2-methylphenyl)ethanesulfonamide; Compound 2.064, which is (R)-2-(5-((3-(isoquinolin-5-ylamino)pyrrolidin-1-yl)methyl)-2-methylphenoxy)ethanol; Compound 2.067, which is (R)-2-(5-((3-(isoquinolin-5-ylamino)pyrrolidin-1-yl)methyl)-2-methoxyphenoxy)ethanol; Compound 2.068, which is (R)-2-(2-fluoro-5-((3-(isoquinolin-5-ylamino)pyrrolidin-1-yl)methyl)phenoxy)ethanol; Compound 2.069, which is (R)-N-(3-((3-(isoquinolin-5-ylamino)pyrrolidin-1-yl)methyl)phenyl)piperidine-1-sulfonamide; Compound 2.073, which is (R)-2-(5-((3-(isoquinolin-5-ylamino)pyrrolidin-1-yl)methyl)-2-methylphenoxy)acetic acid; Compound 2.076, which is (R)-N-(5-((3-(iso quinolin-5-ylamino)pyrrolidin-1-yl)methyl)-2-methylphenyl)methanesulfonamide; Compound 2.077, which is (R)-N-(5-((3-(isoquinolin-5-ylamino)pyrrolidin-1-yl)methyl)-2-methylphenyl)-N′,N′dimethylaminosulfamide; Compound 2.078, which is (R)-N-(3-((3-(isoquinolin-5-ylamino)pyrrolidin-1-yl)methyl)-2-methylphenyl)methanesulfonamide; Compound 2.079, which is (R)-N-(3-((3-(isoquinolin-5-ylamino)pyrrolidin-1-yl)methyl)-2-methylphenyl)-N′,N′ dimethylaminosulfamide; Compound 2.082, which is (R)-N-(1-((2-(methylthio)pyrimidin-4-yl)methyl)pyrrolidin-3-yl)isoquinolin-5-amine; Compound 2.096, which is (R)-N-(3-((3-(isoquinolin-5-ylamino)pyrrolidin-1-yl)methyl)-2-methoxyphenyl)methanesulfonamide; Compound 2.097, which is (R)-N-(3-((3-(isoquinolin-5-ylamino)pyrrolidin-1-yl)methyl)-2-methoxyphenyl)-N′,N′dimethylaminosulfamide; or Compound 2.099, which is (R)-2-(5-((3-(isoquinolin-5-ylamino)pyrrolidin-1-yl)methyl)-2-methylphenoxy)acetamide.

16. The method according to claim 15, wherein said compound is 1.074, 1.075, 1.091, 1.107, 1.123, 1.124, 1.152, 1.153, 1.161, 1.162, 1.165, 1.197, 1.212, 1.213, 1.215, 1.076, 1.077, 1.093, 1.106, 1.108, 1.109, 1.127, 1.157, 1.158, 1.159, 1.176, 1.185, 1.186, 1.195, 1.2, 1.206, 1.208, 1.217, 1.219, 1.223, 1.229, 1.233, 1.236, 1.237, 1.238, 1.239, 1.249, 1.253, 2.058, 2.059, 2.06, 2.066, 1.258, or 1.262.

17. A method for treating pulmonary diseases in patients who had reduced responsiveness to beta adrenergic receptor agonist treatment, comprising the steps of:
identifying a patient suffering from a pulmonary disease, the patient had reduced responsiveness to treatment with a beta adrenergic receptor agonist but has regained responsiveness to the beta adrenergic receptor agonist after a the combined treatment with the beta adrenergic receptor agonist and a corticosteroid, and
administering to the patient an effective amount of a Rho kinase inhibitor compound, wherein said pulmonary disease is selected from the group consisting of: asthma, chronic obstructive pulmonary disease, respiratory tract illness caused by respiratory syncytial virus infection, bronchiectasis, alpha-1-antitrypsin deficiency, lymphangioleiomyomatosis, cystic fibrosis, bronchiolitis or wheezing caused by agents other than respiratory syncytial virus, chronic bronchitis, and occupational lung diseases.

18. The method according to claim 17, wherein said Rho kinase inhibitor compound is a compound of Formula II.

19. The method according to claim 17, wherein said administering is local administering of the Rho kinase inhibitor compound to the lung of the patient.

20. The method according to claim 17, wherein said pulmonary disease is asthma or chronic obstructive pulmonary disease.
